(19) United States
(12) Patent Application Publication
(10) Pub. No.: US 2026/0240842 A1
(43) Pub. Date: Aug. 20, 2026

(54) METHODS TO PREVENT THERAPY RESISTANCE IN MELANOMA VIA BLOCKING GENOMIC INSTABILITY

(71) Applicant: THE REGENTS OF THE UNIVERSITY OF CALIFORNIA, OAKLAND, CA (US)

(72) Inventor: Roger S. Lo, LOS ANGELES, CA (US)

(73) Assignee: THE REGENTS OF THE UNIVERSITY OF CALIFORNIA, OAKLAND, CA (US)

(21) Appl. No.: 18/838,853

(22) PCT Filed: Jan. 25, 2023
(86) PCT No.: PCT/US2023/061285

§ 371 Date: Aug. 15, 2024

Related U.S. Application Data
(60) Provisional application No. 63/268,028, filed on Feb. 15, 2022.

(51) Int. Cl. A61K 31/506; A61K 31/437; A61K 31/4523; A61K 31/519; A61K 39/00; A61K 39/395; A61P 35/00

(52) U.S. Cl. CPC A61K 31/506; A61K 31/437; A61K 31/4523; A61K 31/519; A61K 39/3955; A61P 35/00; A61K 2039/507

(57) ABSTRACT

Compositions and methods for enhancing cancer therapy and the efficacy of the treatment of melanoma, particularly by blocking genomic instability, including chromothriptic genomic instability, to prevent therapy resistance by administering to the subject an effective amount of a DNA-PKcs inhibitor (DNA-PKi) and/or a PARP1/2 inhibitor (PARPi). The subject may be treated with one or more mitogen-activated protein kinase (MAPK) inhibitors (MAPKi), and optionally, one or more anti-PD-1/L1 antibodies as anti-melanoma therapy. Also provided is a method of inhibiting acquired resistance to MAPK inhibitor therapy in a subject in need thereof, as well as a method of inhibiting chromothripsis in a subject in need of MAPKi therapy, by administering to the subject an effective amount of a DNA-PKi and/or a PARPi. The DNA-PKi and/or a PARPi can be administered concomitantly with a MAPKi.

### STATEMENT REGARDING FEDERALLY SPONSORED RESEARCH

This invention was made with government support under Grant Numbers CA176111 and CA168585, awarded by the National Institutes of Health. The government has certain rights in the invention.

This application claims benefit of U.S. provisional patent application No. 63/268,028, filed Feb. 15, 2022, the entire contents of which are incorporated by reference into this application.

### REFERENCE TO A SEQUENCE LISTING

The content of the XML file of the sequence listing named “UCLA286_Seq” which is 12 kb in size was created on Jan. 24, 2023, and electronically submitted herewith the application is incorporated herein by reference in its entirety.

### BACKGROUND

Cancer usually takes years if not decades to develop and spread or become metastatic. When the skin cancer, metastatic cutaneous melanoma, is treated with a type of therapy targeted at an Achilles-heel, pro-growth and -survival pathway (so-called MAPK-targeted therapy), it typically withers away so dramatically that a cure seems imminent. Yet, in a fairly predictable average timeline of months, the cancer regrows and spreads again in a way that is more aggressive than before. This phenomenon, referred to as acquired drug resistance, strongly limits the life-saving power of most if not all our powerful, modern, precision cancer medicines.

MAPK-targeted therapy has been approved to treat the approximately 50 percent of patients with metastatic melanoma that carries a so-called BRAF mutation. In this subset of patient, MAPK-targeted therapy urgently needs to be improved or combined with a new class of drugs that can prevent clinical relapses. MAPK-targeted therapy has been tested in the approximately 20 percent of patients with melanoma that carries a so-called NRAS mutation. However, the cancer develops resistance to the experimental therapy so quickly that MAPK-targeted therapy was not approved to treat such patients. Thus, there is an urgent need to develop a combination therapy, consisting of MAPK-targeted therapy plus another class of drugs, that would offer effective targeted therapy for patients with NRAS-mutated melanoma.

### SUMMARY

The methods described herein meet these needs and more by providing compositions and methods for enhancing cancer therapy and the efficacy of the treatment of melanoma, particularly by blocking genomic instability, including chromothriptic genomic instability, to prevent therapy resistance. In the extant literature, commonly proposed strategies to overcome acquired resistance of melanoma to MAPK-targeted therapy revolve around targeting altered phenotypes to reverse the resistant phenotype. The methods described herein target the mechanism of melanoma evolution of resistance on MAPK-targeted therapy to prevent such resistance. The studies described herein reveal a number of relevant mechanisms, summarized in the following paragraphs, that can inform the therapeutic strategy.

Advanced melanoma before therapy had evolved extensively via chromothripsis. Moreover, after MAPK-targeted therapy and upon acquired resistance, further, recurrent, and even more extensive chromothripsis had taken place. Chromothripsis that arose during therapy encompassed resistance-driver and other potentially driver amplicons, resistance-specific translocations, enriched mutational burden, and altered mutational signatures.

Acquired-resistant melanoma genomes accumulated amplicons within complex genomic rearrangements (CGRs) and extrachromosomal DNAs (ecDNAs). CGR- and ecDNA-amplicons harbored bona fide resistance-driver and DNA repair genes (among others including non-coding RNAs). Breakpoint-sequence analysis of resistance-specific CGR- and ecDNA-amplicons and chromothriptic and non-chromothriptic translocations indicated non-homologous end-joining (NHEJ) as the key pathway of resistance evolution or resistance-specific chromothripsis (genomic instability promoted by DNA double-stranded break or DSB repair). Additional pathways consisted of homologous recombination repair (HRR) and microhomology-mediated end joining (MMEJ, aka alternative NHEJ).

Inhibiting DNA-PKc (key to NHEJ) and PARP1/2 (key to HRR and MMEJ) individually prevented emergence of acquired MAPKi resistant clones in human melanoma cell lines. The former as a single-agent is effective in more cell lines undergoing MAPKi treatment. Co-inhibition of DNA-PKc and PARP1/2 led to additive or synergistic suppression of acquired MAPKi resistance in human melanoma cell lines. Inhibiting DNA-PKc during the early phase of MAPKi treatment was more effective than during the late phase of MAPKi treatment in preventing acquired resistance. Inhibition of DNA-PKc in vivo in patient-derived xenografts of melanoma prevented the acquisition of MAPK inhibitor resistance. The above discoveries have been made in both BRAF V600 mutant and NRAS mutant melanoma.

Other components of the NHEJ, HRR, and MMEJ DSB repair pathways represent rational targets to prevent chromothripsis and/or the evolution of resistance during MAPK-targeted therapy in melanoma (and potentially other human cancers). Components of the NHEJ, HRR, and MMEJ DSB repair pathways represent rational targets to prevent chromothripsis-driven melanoma metastasis from primary disease. Detection of a specific set of chromothriptic DNAs in the tumor, circulating tumor cells, and/or circulating cell-free tumor DNAs may indicate the early emergence of therapy resistance or more aggressive primary disease (i.e., such detection may have prognostic values in the diagnosis of primary cutaneous melanoma).

In some embodiments, described herein is a method of enhancing anti-melanoma therapy in a subject in need thereof, the method comprising administering to the subject an effective amount of a DNA-PKcs inhibitor (DNA-PKi) and/or a PARP1/2 inhibitor (PARPi). In some embodiments, the DNA-PKi is NU7026. In some embodiments, the PARPi is ABT888 (veliparib). In some embodiments, the DNA-PKi is NU7026 and/or AZD7648, In some embodiments, the PARPi is ABT888 (veliparib), olaparib, iniparib, niraparib, talazoparib, AZD2461, and/or rucaparib In some embodiments, the administering comprises administering both a DNA-PKi and a PARPi.

In some embodiments, the subject is treated with one or more mitogen-activated protein kinase (MAPK) inhibitors (MAPKi), and optionally, one or more anti-PD-1/L1 antibodies as anti-melanoma therapy. In some embodiments, the MAPK inhibitor(s) or MAPK inhibitor(s) plus anti-PD-1/L1 antibodies is administered concomitantly with, prior to, and/or subsequent to the administering of the DNA-PKi and/or PARPi. In some embodiments, the MAPK inhibitor is selected from: Vemurafenib, Dabrafenib, Encorafenib, Trametinib, Binimetinib, and Cobimetinib, as well as type II RAF inhibitors or pan-RAF inhibitors, such as BGB-283, BGB-3245, DAY101/TAK-580, KIN-2787, and LXH254. In some embodiments, the MAPK inhibitor is a KRAS-G12C inhibitor, such as sotorasib (also known as AMG 510 or Lumakras) or adagrasib (also known as MRTX849).

Also provided is a method of inhibiting acquired resistance to MAPK inhibitor therapy in a subject in need thereof. In some embodiments, the method comprises administering to the subject an effective amount of a DNA-PKi and/or a PARPi. Additionally provided is a method of inhibiting chromothripsis in a subject in need of MAPKi therapy. In some embodiments, the method comprises administering to the subject an effective amount of a DNA-PKi and/or a PARPi. In some embodiments, the DNA-PKi and/or a PARPi is administered concomitantly with a MAPKi.

In some embodiments, the subject is human. In some embodiments, the subject has been diagnosed with melanoma.

In some embodiments, the subject is treated with one or more mitogen-activated protein kinase (MAPK) inhibitors, and optionally, one or more anti-PD-1/L1 antibodies as anti-melanoma therapy. In some embodiments, the MAPK inhibitor(s) or MAPK inhibitor(s) plus anti-PD-1/L1 antibodies is administered concomitantly with, prior to, and/or subsequent to the administering of the DNA-PKi and/or a PARPi. Typically, the DNA-PKi and/or a PARPi is administered concomitantly with the MAPKi during the initial phase of MAPKI treatment when the tumor cells have slowed in their cycling or proliferative capacity. This initial phase is typically one to three weeks in duration, such as, in some embodiments, 10 days. In some embodiments, the initial treatment phase is one to two months in duration. In some embodiments, the MAPK inhibitor is selected from: a type I RAF inhibitor a MEK inhibitor; a type II RAF inhibitor, a pan-RAF inhibitor, a KRAS-G12C inhibitor; and a combination of the above.

### BRIEF DESCRIPTION OF THE DRAWINGS

FIGS. 1A-1B. Landscape of ecDNAs and CGRs in patient-matched MAPKI-sensitive and acquired-resistant melanoma. Chromosomal distribution of ecDNA- and CGR-amplicons in three cohorts: BRAF^{V600MUT} clinical cohort (baseline tumors, n=8; resistant tumors, n=11; patients, n=8), RAM tissues (sensitive tumor, n=1; resistant tumors, n=12; deceased patients, n=3) and PDXs (vehicle-treated or sensitive, n=6; resistant tumors, n=12; patients of origin, n=6). Os and Xs, ecDNAs and CGRs, respectively; sizes, numbers of copy number variant segments (minimal copy number of 4.5; reference sizes shown). Pre and post tumors from Pt10 and Pt11 not shown, given no detection of ecDNAs and CGRs.

FIGS. 2A-2I. Genes amplified by ecDNAs and CGRs in acquired MAPKi resistance and enriched pathways. FIGS. 2A-2D: Structural variant view of reconstructed amplicons in representative acquired MAPKi-resistant melanoma tumors in the clinical (2A), RAM (2B), BRAF^{V600MUT} PDX (2C), and NRAS^{MUT} PDX (2D) cohorts. Horizontal lines indicate genomic segments with similar copy numbers and genes. Short vertical lines and arcs indicate discordant read pairs linking two amplicons via a structural variant junction. Long vertical lines indicate break ends that map from amplicon into low-complexity regions which cannot be traced further. Arrows point out resistance-driver genes within ecDNAs. DD-DP, Double Drug (BRAFi+MEKi)-Disease Progression. FIG. 2E: Representative images of DNA-FISH showing indicated patient-matched tumors (n=3 pairs) and copy numbers of BRAF or NRAS in either vehicle-treated/MAPKi-sensitive (top) or acquired MAPKi-resistant (middle) melanoma. CEN, centromere; DAPI, nuclear stain. Ruler, 25 mm. Quantification of DNA-FISH (bottom) from 40 nuclei per tumor; results shown as mean±S.D. P values (unpaired two-tailed Students t-test): ***, p<0.001. FIG. 2F: Structural variant view of reconstructed amplicons in clinically acquired resistance showing XRCC2 as a putative resistance-driving gene. FIG. 2G: Number of ecDNA-amplified genes (i) shared between pre/sensitive and post/acquired-resistant tumors, (ii) specifically in pre/sensitive tumors, (iii) specifically in post/acquired-resistant tumors, and (iv) in post/acquired-resistant tumors shared with CGR-amplified genes in pre/sensitive tumors. Cohorts: clinical (n=8 patients), RAM (n=1 patient), and PDX models (n=6 patients). Patients without sensitive (n=2) and without ecDNAs and CGRs detected (n=2) excluded. FIG. 2H: As in 2G, except showing the number of CGR-amplified genes in the same (i) to (iii). (iv) Number of CGR-amplified genes in post/acquired-resistant tumors shared with ecDNA-amplified genes in pre/sensitive tumors. FIG. 2I: Pathway enrichment analysis of genes amplified by ecDNAs and/or CGRs exclusively in acquired resistant tumors (n=8). Only tumors with sufficient genes for enrichment analysis were analyzed.

FIGS. 3A-3E. Associations of chromothripsis, ecDNAs and CGRs with mutagenic and double-stranded DNA repair pathways. FIG. 3A: Pattern of overlap or non-overlap between ecDNA+CGR and chromothriptic genomic spans within individual sensitive (n=17; top) and acquired-resistant (n=41; bottom) melanoma tumors. Sizes (Mb) of chromothriptic regions in light gray horizontal bars; of ecDNAs+CGRs overlapping with chromothripsis in medium gray; and of ecDNAs+CGRs non-overlapping with chromothripsis in dark gray. P value (Fisher's exact test) of non-random overlap between chromothriptic and ecDNA+CGR regions, ***, p<0.00001. FIG. 3B: Single base substitution (SBS) signatures of chromothriptic genomes of MAPKi-sensitive/naive (left, n=16) versus -resistant (right, n=31) melanoma tumors. Excluded from analysis, acquired-resistant tumors without patient-matched sensitive tumors and tumors without chromothripsis. FIG. 3C: Top, SBS signatures of ecDNAs+CGRs of MAPKi-sensitive/naive (left, n=12) versus -resistant (right, n=28) melanoma tumors. Bottom, SBS signature enrichment scores shown by both heat scale and dot size. Increasing dot sizes indicate increasing enrichment of indicated signatures within ecDNAs+CGRs compared to regions devoid of these events (score>1). Enrichment score=1 is considered as the cut-off; only scores>1 and <1 are shown. Excluded from analysis, acquired-resistant tumors without patient-matched sensitive tumors and tumors without ecDNAs and CGRs. FIG. 3D: Breakpoint-junctional sequence analysis of ecDNAs+CGRs inferring indicated DNA DSB repair processes: NHEJ, alt-NHEJ, and HRR. Amplicons from all three cohorts of tumors combined for analysis (sensitive, n=17; resistant, n=41). Homologous sequences of size 0-1 bp (NHEJ), 2-8 bp (alt-NHEJ), and >8 bp and large insertions (HRR). P value (Kruskal-Wallis test)=6.35e-16. FIG. 3E: As in 3D, except only for on resistant tumor-derived ecDNA- and CGR-amplicons that harbor BRAF (n=11), NRAS (n=5), HRAS (n=2), and EGFR (n=4) genes.

FIGS. 4A-4U. DNA-PKi and/or PARPi co-treatment prevents acquired MAPKi-resistance in human melanoma cell lines. FIGS. 4A to 4O: Long-term clonogenic growth of isogenic parental and acquired MAPKi-resistant BRAF^{V600MUT} (4A to 4H) or NRAS^{Q61MUT} (4I to 4O) human melanoma cell lines showing acquired-resistant colonies to BRAFi+MEKi (PLX4032 at 0.5 mM, AZD6244 at 0.5 mM in 4A and 4G; PLX4032 at 0.25 mM, AZD6244 at 0.25 mM in 4D; PLX4032 at 1 mM, AZD6244 at 1 mM in 4B, 4C, 4E, 4F and 4H) or MEKi (trametinib at 0.005 mM in I and 0.01 mM in 4J and 4N; trametinib at 0.1 mM in 4K, 4M and 4O) and their suppression by indicated co-treatments with DNA-PKi (NU7026) and/or PARPi (ABT888) at indicated concentrations. Top, representative cultures; bottom, quantifications over n=4 fields (mean±SD). Data representative of 2 to 4 independent repeats. Seeding densities (cells/well in 6-well dishes) and culture durations (days): (4A) 5,000; 31, (4B) 1,000; 17, (4C) 5,000; 18, (4D) 40,000; 40, (4E) 5,000; 14, (4F) 5,000; 12, (4G) 20,000; 30, (4H) 20,000; 21, (4I) 40,000; 30, (4J) 50,000; 31, (4K) 5,000; 17, (4L) 5,000; 22, (4M) 5,000; 16, (4N) 20.000; 16, and (4O) 5,000; 16. Data representative of 2 to 4 independent repeats. FIGS. 4P and 4Q: Western blot, MTT assay (upper), and clonogenic growth assay (lower) of M245 cells transduced by lentivirus harboring shVector control or shRNAs of PRKDC (4P) or LIG4 (4Q). TUBULIN, loading control. Clonogenic growth with vehicle (14 days, 5,000 cells/well in 6-well dishes) or MEKi (29 days, 50,000 cells/well in 6-well dishes) treatments. MEKi, trametinib at 0.01 μM. Representative results of two independent experiments; quantification of n=4 fields (mean±SD). FIGS. 4R and 4S: As in 4A and 4J, respectively, except DNA-PKi and/or PARPi co-treatments were performed in M229 (4R) and M245 (4S) at a later stage of MAPKi treatment (see timepoint schema). Inhibitor concentrations for M229: BRAFi+MEKi (PLX4032 at 0.5 μM, AZD6244 at 0.5 μM), NU7026 (8 μM), ABT888 (4 μM) and NU7026+ABT888 (4 μM+4 μM). Inhibitor concentrations for M245: MEKi (trametinib at 0.01 μM), NU7026 (5 μM), ABT888 (1 μM) and NU7026+ABT888 (5 μM+1 μM). Left, representative cultures; right, quantifications over n=4 fields (mean±SD). Data representative of 2 independent repeats. FIGS. 4T and 4U: As in 4A and 4J, respectively, except DNA-PKi and/or PARPi co-treatments were performed in M229 (4T) and M245 cells (4U) at the same concentrations in R and S for the entire durations (day 1-28 in 4T; day 1-30 in 4U), the first half (day 1-14 in T; day 1-15 in 4U) or the second half (day 14-28 in 4T: day 15-30 in 4U) of the total treatment course. Top, representative cultures; bottom, quantifications over n=4 fields (mean±SD). Data representative of 2 independent repeats. P values (one-way ANOVA followed by Tukey's multiple comparison test) comparing indicated cultures versus MAPKi-only cultures (4A to 4O and 4R to 4U) or shVector culture (4P and 4Q): *, p<0.05; **, p<0.01: ***, p<0.001.

FIGS. 5A-5H. Resistance-driver ecDNAs and HSRs dynamically track with resistance and DNA-PKi prevents size-expansion of ecDNAs and CGRs early on MAPKi treatment. FIGS. 5A to 5C, Metaphase DNA-FISH of paired parental and acquired resistant BRAF^{V600MUT} (5A and 5B) or NRAS^{Q61MUT} (5C) cell lines without or with drug withdrawal showing ecDNA- or HSR-amplicons harboring BRAF, RAF1 or NRAS. Left, representative images; right, quantifications per cell. Ruler, 15 μm. P values (Unpaired two-tailed Student's t-test): *, p<0.05; **, p<0.01; ***, p<0.001. FIGS. 5D to 5F: Three- (upper) and six-(lower) day MTT assay of indicated cell lines treated with graded concentrations of BRAFi+MEKi (5D and 5E) or MEKi (5F). Cell viability was normalized to respective DMSO/vehicle group. Inner brackets, comparisons between acquired-resistant sub-lines without and with drug withdrawal. Outer brackets, comparisons between acquired-resistant sub-lines and their isogenic parental cell lines. P values (two-way ANOVA test): *, p<0.05; **, p<0.01; ***, p<0.001. FIG. 5G: Representative images (left) and quantification (right) of metaphase DNA-FISH showing HSR harboring BRAF in M249 cells treated with vehicle or BRAFi+MEKi, with or without NU7026 and/or ABT888 for 31 days. BRAFi+MEKi (PLX4032 at 0.25 μM, AZD6244 at 0.25 μM), NU7026 (4 μM), ABT888 (2 μM) and NU7026+ABT888 (4 μM+2 μM). CEN, centromere; DAPI, nuclear stain. Ruler, 15 μm. P values (unpaired two-tailed Student's t-test): *, p<0.05; **, p<0.01; ***, p<0.001. FIG. 5H: Average total genomic spans of treatment-specific ecDNAs+CGRs in M229 and M245 cell lines (background ecDNA+CGR spans detected in vehicle-treated cells were filtered). BRAFi+MEKi (PLX4032 at 1 mM, AZD6244 at 1 mM for M229) or MEKi (trametinib at 0.02 mM for M245), DNA-PKi (NU7026 at 8 mM for both cell lines).

FIGS. 6A-6J. DNA-PKi co-treatment prevents acquired MAPKi-resistance in KRAS^{G12} human pancreatic ductal adenocarcinoma and non-small cell lung carcinoma cell lines. FIGS. 6A to 6J: Long-term clonogenic growth of PDAC (MIAPaCa-2, XWR200) (6A to 6F) or NSCLC (H358, H2122) (6G to 6J) cell lines showing acquired-resistant colonies to MEKi alone, type II RAFi+MEKi, or KRAS G12Ci+MEKi and their suppression by indicated co-treatments with DNA-PKi (NU7026) and/or PARPi (ABT888) at indicated concentrations. Top, representative cultures; bottom, quantifications over n=4 fields (mean±SD). Data representative of 2 independent repeats. Seeding densities (cells/well in 6-well dishes) and culture durations: MIAPaCa-2 (10,000; 30 days for 6A-6C), XWR200 (150,000; 23 days for 6D, 30 days for 6E, 26 days for 6F), H358 (40,000; 14 days for 6G, 29 days for 6H, 26 days for 6I), H2122 (20,000, 22 days for 6J). Concentrations of type II RAFi (BGB-283)+MEKi (trametinib) or KRAS G12Ci (AMG510 or MRTX849)+MEKi (trametinib) or MEKi (trametinib): (6A) 0.5 mM+0.01 mM, (6B) 0.02 mM+0.01 mM, (6C) 0.01 mM+0.01 mM, (6D) 0.1 mM+0.001 mM, (6E) 0.005 mM+0.001 mM, (6F) 0.001 mM+0.001 mM, (6G) 0.2 mM+0.001 mM, (6H) 0.005 mM+0.001 mM, (6I) 0.002 mM+0.001 mM, (6J) 0.02 mM. Data representative of 2 to 4 independent repeats. P values (one-way ANOVA followed by Tukey's multiple comparison test) comparing indicated cultures versus MAPKi treatment only cultures: *, p<0.05; *, p<0.01; **, p<0.001.

FIGS. 7A-7G. DNA-PKi co-treatment with MAPKi reduces the size of ecDNAs and CGRs and prevents acquired-resistance in vivo. FIGS. 7A to 7E: Measurements of tumor volumes (left) and body weights of mice (right) in two BRAF^{V600MUT} (7A and 7B) and three NRAS^{MUT} (7C to 7E) cutaneous melanoma PDX models. Vehicle or indicated treatments initiated on well-established tumors on day 33 (7A), 48 (7B), 34 (7C), 43 (7D), and 42 (7E) after tumor fragment implantation, as marked by upside down dark gray triangles. Dosage of inhibitors DNA-PKi (NU7026), BRAFi (vemurafenib), and MEKi (trametinib) (mg/kg/day): (7A and 7B) DNA-PKi, 8; BRAFi, 90; MEKi, 0.7; (7C) DNA-PKi, 10; MEKi, 3; (7D) DNA-PKi, 6; MEKi, 5; and (7E) DNA-PKi, 8 (stopped on day 67 when in combination with MEKi, as marked by an upside down light gray triangle); MEKi, 3. One tumor per mouse; number of mice per experimental group (from top to bottom): (7A) 5, 5, 7, 7; (7B) 4, 5, 8, 8; (7C) 5, 5, 8, 5; (7D) 3, 4, 8, 8; and (7E) 5, 5, 6, 9. Results shown as mean t SEM. P values, Student's t-test. Body weights shown as average values. FIG. 7F: As in FIG. 5B (BRAFi+MEKi treatment group), except at the earliest time of acquired resistance (day 84, marked by an upside down light gray triangle), tumors or mice were divided into two groups (n=5 each). Group one continued on BRAFi+MEKi treatments. Group two received DNA-PKi (8 mg/kg/day) in combination with BRAFi+MEKi treatments. FIG. 7G: Average total spans of ecDNAs+CGRs specific to MEKi-treated versus MEKi+DNA-PKi-treated PDXs (background ecDNAs+CGRs detected in vehicle-treated tumors were filtered) in Mel_PDX16 (BRAFi+MEKi-treated, n=3; BRAFi+MEKi+DNA-PKi-treated, n=3), Mel_PDX27 (BRAFi+MEKi-treated, n=3; BRAFi+MEKi+DNA-PKi (8 mg/kg/day)-treated, n=3; BRAFi+MEKi+DNA-PKi (10 mg/kg/day)-treated, n=2), Mel_PDX1 (MEKi-treated, n=2; MEKi+DNA-PKi-treated, n=2), Mel_PDX2 (MEKi-treated, n=2; MEKi+DNA-PKi-treated, n=2), and Mel_PDX4 (MEKi-treated, n=2; MEKi+DNA-PKi-treated, n=3). Tumors were collected for analysis on days 6 (Mel_PDX16), 7 (Mel_PDX27), 11 (Mel_PDX1), 9 (Mel_PDX2), and 8 (Mel_PDX4) after initiating treatments.

FIGS. 8A-8H. Associations of CGRs and ecDNAs with ploidy, expression, and enhancers. FIG. 8A: Tumor volumes of individual tumors, either vehicle-treated (n=6, dashed lines) or MAPKi-treated (n=12, solid lines) melanoma PDXs until acquired-resistant tumors emerged. Five NRAS^{MUT} (Mel_PDX1, Mel_PDX2, Mel_PDX3, Mel_PDX4, Mel_PDX6) and one BRAF^{V600MUT} (Mel_PDX27) tumors were treated with trametinib at 5 mg/kg/d. FIG. 8B: Pairwise comparisons of the numbers of ecDNAs and CGRs in MAPKi-sensitive and acquired-resistant tumors (n=15). Averages used for multiple resistant tumors derived from the same patient. Excluded from analysis, resistant tumors without patient-matched MAPKi-sensitive tumors and tumors without detected ecDNAs or CGRs. P value (paired Student's t-test): *, p<0.05. FIG. 8C: Number of CGR versus ecDNA segments in all tumors (n=58). P value (Student's t-test): ***, p<0.001. FIG. 8D: Numbers of CGR and ecDNA segments in each of three tumor cohorts. P value (Kruskal-Wallis test): **, p<0.01. FIG. 8E: Ploidy versus the numbers of CGR and ecDNA segments. Both MAPKi-sensitive/-naive and acquired MAPKi-resistant tumors were included together. P value (Spearman correlation): rₛ=0.44, p=0.0004. FIG. 8F: Changes in transcript levels (measured by RNA-seq) and associated CNV values of MAPKi resistance-driver genes (BRAF, NRAS, MYC, EGFR, HRAS and RAC1, as shown in FIG. 1A) in acquired MAPKi-resistant (versus MAPKi-sensitive/-naive) tumors. FIG. 8G: Enhancers on ecDNAs and their cis or trans target genes in Pt2-DP1 (EZH2 and XRCC2 in chr7), Pt3-DP1 (BRAF in chr7), RAM12.01-Brain-DD-DP10 (BRAF in chr7), Mel_PDX1 (NRAS in chr1), Mel_PDX2 (NRAS and ATP1A1 in chr1), Mel_PDX4 (RAC1 in chr7). Super-enhancers are indicated with a star. FIG. 8H: H3K27 acetylation marks in Mel_PDX27-R2 (with BRAF) and Pt9-DD-DP2 (with MYC). Arc connecting the endpoints indicates ecDNA and downward arrow indicates H3K27 acetylation peaks in the enhancers of BRAF and MYC.

FIGS. 9A-9F. Analysis of chromothripsis, ecDNAs, and CGRs in MAPKi-sensitive/-naive versus -resistant melanoma. FIG. 9A: Chromothriptic genomic regions in patient-matched MAPKi-sensitive versus acquired resistant genomes (n=58). FIG. 9B: Frequencies of chromothriptic events exclusively observed in BRAF^{MUT} MAPKi-sensitive (n=11), BRAF^{MUT} acquired-resistant (n=17), and NRAS^{MUT} acquired-resistant (n=9) tumors. FIG. 9C: Ratios of tumor mutational burden (somatic SNVs) in regions within and without chromothripsis (n=58). FIG. 9D: DBS and ID signatures of chromothriptic genomes in MAPKi-sensitive/naive (left) versus -resistant (right) melanoma tumors. Excluded from analysis, resistant tumors without patient-matched sensitive tumors. FIG. 9E: SBS signatures detected in ecDNAs (left; MAPKi-sensitive/-naive, n=5; acquired resistant, n=19) and CGRs (right; MAPKi-sensitive/-naive, n=11; acquired resistant, n=24). Excluded from analysis, resistant tumors without patient-matched sensitive tumors and tumors without detected ecDNAs and CGRs. FIG. 9F: Proportions of SBS signatures associated with APOBEC family of cytidine deaminases (top, SBS2 and SBS13) and defective HRR (bottom, SBS3) in ecDNAs (n=19) and CGRs (n=24) of acquired resistant tumors. P value (Wilcoxon rank sum test): *, p<0.05.

FIGS. 10A-10C. (Right) Examples of ecDNAs harboring BRAF, BRAF, and NRAS genes from PDX tumors (10A) Mel_PDX27-R2 (SEQ ID NOs: 7, 8, respectively), (10B) Pt3-DP1 (SEQ ID NOs: 9, 10, respectively), and (10C) Mel_PDX2-R2 (SEQ ID NOs: 11, 12. respectively) and their breakpoint junctions (shown in detail on the left), without and with micro homologous sequences (AAAT bases in dark gray) and insertions (CCTTCCCCTATGG bases in medium gray). Sequences in ecDNAs are shown in black and adjacent DNA sequences in light grey. Inferred double-stranded DNA break repair mechanisms shown (middle). Non-homologous end-joining (NHEJ), alternate-NHEJ (Alt-NHEJ), and homologous recombination repair (HRR). FIG. 10B: Changes in the percentages of inferred DNA DSB repair processes (NHEJ, alt-NHEJ, HRR) in BRAF^{V600MUT} (top; n=10 sensitive tumors; n=29 resistant tumors) and NRAS^{MUT} (bottom; n=5 sensitive tumors; n=9 resistant tumors) melanoma. Inferences based on breakpoint-junctional sequence analysis of ecDNAs+CGRs. P value (within group, Kruskal-Wallis test): ***, p<0.001 and P value (across the groups, BRAF^{V600MUT} versus NRAS^{MUT} tumors; Student's t-test): *, p<0.05. FIG. 10C: Breakpoint-junctional sequence analysis of ecDNAs (shaded) and CGRs (black) inferring indicated double-stranded DNA break repair processes. Amplicons from MAPKi-sensitive (left, n=17) and acquired-resistant (right, n=41) tumors in all three cohorts of tumors combined for analysis. P value (Wilcoxon rank sum test): *, p<0.05; **, p<0.01; ***, p<0.001.

FIGS. 11A-11G. Single-agent inhibitory potencies in human melanoma, PDAC, and NSCLC clonogenic growth assays. FIGS. 11A to 11G: Long-term clonogenic growth of indicated BRAF^{V600MUT} or NRAS^{Q61MUT} human melanoma cell lines (11A to 11E) and KRAS^{G12C} human pancreatic ductal adenocarcinoma and non-small cell lung carcinoma cell lines (11F and 11G) treated with the indicated concentrations of DNA-PKi (NU7026 in 11A; VX984 in 11C; AZD7648 in 11D) or PARPi (ABT888 in 11B; olaparib in 11E). Cells were seeded at 5,000 cells/well in 6-well dishes (11A to 11E). Culture durations (days): 9, M207, M245 (11C, 11E); 13, M207, M245, M249 (11A, 11B, 11D); 15, M202; 17, M229; 18, M395. Seeding densities (cells/well in 6-well dishes) and culture durations (days) for 11F and 11G: MIAPaCa-2 (2,000;17), XWR200 (150,000; 14), H358 (20,000; 10), H2122 (10,000, 10).

FIGS. 12A-12F. DNA-PKi and/or PARPi co-treatment prevents acquired MAPKi-resistance in human melanoma cell lines. FIGS. 12A to 12F: Long-term clonogenic growth of BRAF^{V600MUT} (12A, 12C and 12E) or NRAS^{Q61MUT} (12B, 12D and 12F) cell lines showing acquired-resistant colonies to BRAFi+MEKi (PLX4032 at 0.5 mM, AZD6244 at 0.5 mM for M229 and M395; PLX4032 at 0.25 mM, AZD6244 at 0.25 mM for M249) or MEKi (trametinib at 0.005 mM for M202 and 0.01 mM for M207 and M245) and their suppression by indicated co-treatments with DNA-PKi (VX984 in 12A and 12B; AZD7648 in 12C and 12D) and/or PARPi (olaparib in 12E and 12F) at indicated concentrations. Top, representative cultures; bottom, quantifications over n=4 fields (mean±SD). Data representative of 2 independent repeats. Seeding densities (cells/well in 6-well dishes) and culture durations (days): M202 (40,000, 30), M207 (20,000, 16), M229 (5,000, 32), M245 (50,000, 31), M249 (40,000, 40), and M395 (20,000, 30). Data representative of 2 to 4 independent repeats. P values (one-way ANOVA followed by Tukey's multiple comparison test) comparing indicated cultures versus MAPKi treatment only cultures: *, p<0.05; **, p<0.01; ***, p<0.001.

FIGS. 13A-13H. In vivo impacts of DNA-PKi when combined with MAPKi. FIGS. 13A and 13B: Long-term follow-up of tumor volumes (13A, BRAFi+MEKi, n=5 tumors; BRAFi+MEKi+DNA-PKi, n=6 tumors) (13B, MEKi, n=6 tumors; MEKi+DNAP-PKi, n=6 tumors) and average mice body weights for 13A and 13B. Related to experiments in FIGS. 7B and 7E, respectively. FIGS. 13C and 13D, Tumor volumes (mean±SEM) of two examples of tumor models treated short-term with vehicle or indicated single versus combined agents. Inhibitors and dosages are identical to those in FIGS. 7C and 7E, respectively. The numbers of tumors and their sizes are shown in the photographs (right) taken at the times of tumor harvest (end points). FIGS. 13E to 13G: Immunofluorescence of p-ERK (13E), p-DNA-PKcs (S2056) (13F), or γH2AX (13G) levels in Mel_PDX1 tumor model, treated with vehicle or indicated single versus combined agents for 11 days (shown in 13C). DAPI, nuclear stain. Scale bar, 50 μm (13E) and 20 μm (13F and 13G). Representative images shown for two tumors analyzed per group. Quantification of p-DNA-PKcs signals obtained by taking the average of positively stained foci per nucleus (13F, representative of 4 fields). Quantification of γH2AX signals analyzed either by percentage of cells with γH2AX foci or percentage of cells with greater than 5 γH2AX foci (13G, representative of 4 fields). P values (Student's t test): *, p<0.05; **, p<0.01. FIG. 13H: Changes in the percentages of inferred DNA double-stranded break repair processes (NHEJ, alt-NHEJ, HRR) due to combined MAPKii+DNA-PKi treatment (versus MAPKi alone). Inferences based on breakpoint-junctional sequence analysis of ecDNAs+CGRs specific to each treatment group. P value (Student's t-test): *, p<0.05 (MEKi+DNA-PKi vs. MEKi).

### DETAILED DESCRIPTION

The invention is based on the unexpected discovery that chromothripsis as well as ecDNA- and CGR-amplicons drive evolution of MAPKi resistance in BRAF^{V600MUT} and NRAS^{MUT} melanoma. Described herein is a novel strategy that targets chromothripsis and ecDNA- and CGR-amplicons to suppress acquired resistance in BRAF^{V600MUT} or NRAS^{MUT} melanoma in response to MAPKi therapy. The examples provided herein show that focal, high-amplitude gene amplifications that drive resistance to MAPKi therapy in melanoma often reside within intra- or inter-chromosomal regions of complex genomic rearrangements (CGRs) or ecDNAs, which can be derived from chromothripsis. As demonstrated herein, co-targeting double-stranded DNA break (DSB) repair proteins selects against evolution of MAPKi resistance.

### Definitions

All scientific and technical terms used in this application have meanings commonly used in the art unless otherwise specified. As used in this application, the following words or phrases have the meanings specified.

As used herein, “combinatorial therapy” means MAPK targeted therapy, anti-CTLA-4 immunotherapy in any combination, with or without anti-PD-1 antibody and/or anti-PD-L1 antibody treatment.

As used herein, “MAPK/ERK kinase (MEK)” refers to a mitogen-activated protein kinase also known as mitogen-activated protein kinase (MAPK) or extracellular signal-regulated kinase (ERK).

MEK, also known as mitogen-activated protein kinase kinase and MAP2K, is a kinase enzyme that phosphorylates mitogen activated protein kinases (MAPKs), ERK, p38 and JNK. Seven MEK subtypes have been identified, all mediate cellular responses to different growth signals.

BRAF (v-raf murine sarcoma viral oncogene homolog B1) is a serine/threonine protein kinase that plays a critical role in the RAS-RAF-MEK-ERK mitogen activated protein kinase (MAPK) cell signalling pathway.

As used herein, “PD-1 (programmed cell death-1)” refers to a receptor expressed on the surface of activated T cells. “PD-L1 and PD-L2” are PD-1 ligands expressed on the surface of dendritic cells, macrophages, or tumor cells. PD-1 and PD-L1/PD-L2 belong to the family of immune checkpoint proteins that act as co-inhibitory factors that can halt or limit activation or persistence of anti-tumor T cell responses.

As used herein, “anti-PD-1 therapy” means treatment with an anti-PD-1 antibody (nivolumab/BMS-936558/MDX-1106, pembrolizumab/MK-3475, Pidilizumab), and/or an anti-PD-L1 antibody (BMS-986559, MPDL3280A, and MED14736).

As used herein, “therapy”, “treatment” or “treating” means any administration of a therapeutic agent according to the present disclosure to a subject (e.g. human) having or susceptible to a condition or disease, such as cancer, for the purpose of: preventing or protecting against the disease or condition, that is, causing the clinical symptoms not to develop; inhibiting the disease or condition, that is, arresting or suppressing the development of clinical symptoms; or relieving the disease or condition that is causing the regression of clinical symptoms. In some embodiments, the term “therapy”, “treatment” or “treating” refers to relieving the disease or condition, i.e. which is causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment. The presence of a genetic mutation or the predisposition to having a mutation may not be alterable. However, prophylactic treatment (prevention) as used herein has the potential to avoid/ameliorate the symptoms or clinical consequences of having the disease engendered by such genetic mutation or predisposition. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.” The term “effective amount” refers to that amount of a therapeutic agent that is sufficient to effect treatment when administered to a subject in need of such treatment. The effective amount will vary depending upon the specific activity of the therapeutic agent being used, the severity of the patient's disease state, and the age, physical condition, existence of other disease states, and nutritional status of the patient. Additionally, other medication the patient may be receiving will affect the determination of the effective amount of the therapeutic agent to administer.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### Methods

Described herein is a method of enhancing anti-melanoma therapy in a subject in need thereof, the method comprising administering to the subject an effective amount of a DNA-PKcs inhibitor (DNA-PKi) and/or a PARP1/2 inhibitor (PARPi). In some embodiments, the DNA-PKi is NU7026. In some embodiments, the PARPi is ABT888 (veliparib). In some embodiments, the DNA-PKi is NU7026 and/or AZD7648, In some embodiments, the PARPi is ABT888 (veliparib), olaparib, iniparib, niraparib, talazoparib, AZD2461, and/or rucaparib. In some embodiments, the administering comprises administering both a DNA-PKi and a PARPi.

In some embodiments, the subject is treated with one or more mitogen-activated protein kinase (MAPK) inhibitors (MAPKi), and optionally, one or more anti-PD-1/L1 antibodies as anti-melanoma therapy. In some embodiments, the MAPK inhibitor(s) or MAPK inhibitor(s) plus anti-PD-1/L1 antibodies is administered concomitantly with, prior to, and/or subsequent to the administering of the DNA-PKi and/or PARPi. In some embodiments, the MAPK inhibitor is selected from: Vemurafenib, Dabrafenib, Encorafenib, Trametinib, Binimetinib, and Cobimetinib, as well as type II RAF inhibitors or pan-RAF inhibitors, such as BGB-283, BGB-3245, DAY101/TAK-580, KIN-2787, and LXH254. In some embodiments, the MAPK inhibitor is a KRAS-G12C inhibitor, such as sotorasib (also known as AMG 510 or Lumakras) or adagrasib (also known as MRTX849), which act by selectively forming a covalent bond with cysteine 12 within the switch-II pocket of KRAS-G12C protein, thereby locking KRAS in the inactive state to arrest cell proliferation.

Also provided is a method of inhibiting acquired resistance to MAPK inhibitor therapy in a subject in need thereof. In some embodiments, the method comprises administering to the subject an effective amount of a DNA-PKi and/or a PARPi. Additionally provided is a method of inhibiting chromothripsis in a subject in need of MAPKi therapy. In some embodiments, the method comprises administering to the subject an effective amount of a DNA-PKi and/or a PARPi. In some embodiments, the DNA-PKi and/or a PARPi is administered concomitantly with a MAPKi.

In some embodiments, the subject is human. In some embodiments, the subject has been diagnosed with melanoma.

In some embodiments, the subject is treated with one or more mitogen-activated protein kinase (MAPK) inhibitors, and optionally, one or more anti-PD-1/L1 antibodies as anti-melanoma therapy. In some embodiments, the MAPK inhibitor(s) or MAPK inhibitor(s) plus anti-PD-1/L1 antibodies is administered concomitantly with, prior to, and/or subsequent to the administering of the DNA-PKi and/or a PARPi. Typically, the DNA-PKi and/or a PARPi is administered concomitantly with the MAPKi during the initial phase of MAPKi treatment when the tumor cells have slowed in their cycling or proliferative capacity. This initial phase is typically one to three weeks in duration, such as, in some embodiments, 10 days. In some embodiments, the initial treatment phase is one to two months in duration. In some embodiments, the MAPK inhibitor is selected from: a type I RAF inhibitor a MEK inhibitor; a type II RAF inhibitor, a pan-RAF inhibitor, a KRAS-G12C inhibitor; and a combination of the above.

### Kit

The invention provides kits comprising one or more therapeutic agents as described herein, such as a DNA-PKi and/or a PARPi, MAPK inhibitor(s), anti-PD-1/L1 antibodies, and optionally, one or more suitable containers containing therapeutic agents of the invention. The kit provides therapeutic agents as compositions, or unit dosage forms and/or articles of manufacture. in some embodiments, the kit further comprises instructions for use in accordance with any of the methods described herein. The kit may further comprise a description of an individual suitable for treatment. Instructions supplied in the kits are typically written instructions on a label or package insert (e.g., a paper sheet included in the kit), but machine-readable instructions are also acceptable. The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

### EXAMPLES

The following examples are presented to illustrate the present invention and to assist one of ordinary skill in making and using the same. The examples are not intended in any way to otherwise limit the scope of the invention.

### Example 1: Blocking Genomic Instability to Prevent Therapy Resistance in Melanoma

This Example demonstrates that, after MAPK inhibitor (MAPKi) therapy in patients and patient-derived xenografts (PDXs), acquired-resistant genomes of metastatic cutaneous melanoma amplify resistance-driver, non-homologous end-joining (NHEJ), and homologous recombination repair (HRR) genes via complex genomic rearrangements (CGRs) and circular extrachromosomal DNAs (ecDNAs). Almost all sensitive and acquired-resistant genomes harbor pervasive chromothriptic regions with disproportionately high mutational burdens and significant overlaps with ecDNA and CGR spans. Recurrently, somatic mutations within ecDNA- and CGR-amplicons enrich for HRR signatures, particularly within acquired-resistant tumors. Regardless of sensitivity or resistance, breakpoint-sequence analysis suggests NHEJ as critical to double-stranded DNA break repair underlying CGR and ecDNA formation. In human melanoma cell lines and PDXs, NHEJ-targeting by a DNA-PKCS inhibitor prevents/delays acquired MAPKi resistance by reducing the size of ecDNAs and CGRs early on combination treatment. Thus, targeting the cause(s) of genomic instability prevents acquired resistance.

Acquired resistance to targeting of oncogenic pathways is the rule rather than the exception. Genetic mechanisms of acquired resistance across malignancies often occur as focal amplifications of resistance-driver genes. Up to now, scientific efforts have focused largely on reversing consequences of resistance evolution, such as acquired vulnerabilities. In contrast, relatively little is known regarding the mechanisms, e.g., genomic instability pathways, that enable rapid resistance evolution in patients treated with pathway-targeted therapies. High-amplitude, focal gene amplifications can confer oncogenic signaling plasticity capable of fine-tuning tumor cell fitness in response to the selective pressure of therapies.

BRAF inhibitor (BRAFi) therapy of BRAF^{V600MUT} metastatic cutaneous melanoma targets the MAPK pathway and leads quickly to acquired resistance (1). Genetic mechanisms of acquired BRAFi resistance most often result in reactivation of the MAPK pathway, which can be suppressed by adding a MEK inhibitor (MEKi) (2-5). However, with combined BRAFi and MEKi therapy of patients with BRAF^{V600MUT} melanoma, less than 20% of patients survive past five years (6). Acquired resistance to BRAFi+MEKi therapy in patients occurs through high-amplitude gene amplifications that, individually or in combination, can reactivate the MAPK pathway (7-9). Hence, there is a need to understand the origins of such genomic instability. For patients with melanoma driven by NRAS mutations, MAPK inhibitor (MAPKi) therapy is currently not available, as MEKi monotherapy is of limited clinical activity (10). However, agents added to MEKi to suppress acquired MEKi resistance may deliver clinically meaningful efficacy (11). Recent analysis of patient-derived xenografts (PDXs) of NRAS^{MUT} melanoma with acquired MEKi resistance points to similar genomic events (i.e., focal amplifications of BRAF^{WT}, CRAF^{WT}, and NRAS^{MUT} genes) as drivers of acquired resistance (11).

In this Example, we test the hypothesis that chromothripsis and derivative amplicons of resistance driver-genes confer melanoma with genetic variants necessary to resist MAPKi therapy. Cutaneous melanoma is a cancer in which the chromothripsis burden is already high without prior targeted therapy, and chromothripsis appears to be a key evolutionary mechanism by which cancer rapidly generates and accumulates highly dynamic structural variants (SVs) (12). SV-related amplicons can be identified as intrachromosomal complex genomic rearrangements (CGRs) and circular extrachromosomal DNAs (ecDNAs, aka double minutes), which may be temporally related structures that confer an added level of genomic-signaling plasticity (13). Earlier studies have already established the prognostic importance of ecDNAs in clinical tumor tissues and the potential relevance of ecDNAs to therapeutic resistance in cancer cell lines (14,15). The non-Mendelian inheritance of ecDNAs, their accessible chromatin, as well as enhancer hijacking and transcriptional hub congregation by ecDNAs, are all mechanisms that facilitate rapid adaptations to extreme stress such as oncogene-targeted therapy (16-19). Finally, iterative cycles of CGR and ecDNA biogenesis drive the amplification of a model resistance gene (DHFR) to the chemotherapy, methotrexate (20).

To test the hypothesis that chromothripsis, CGRs, and ecDNAs play key roles in the evolution of MAPKi resistance in clinical melanoma, we analyzed by whole-genome sequencing (WGS) three tumor cohorts (along with patient-matched normal tissues): (i) patient-matched pre-MAPKi and acquired MAPKi-resistant melanoma tumors from patients with BRAF^{V600MUT} melanoma, (ii) acquired MAPKi-resistant melanoma metastatic to multiple organ sites (including the brain) from rapid autopsies of deceased patients with BRAF^{V600MUT} melanoma, and (iii) acquired MEKi-resistant melanoma and untreated melanoma from BRAF^{V600MUT} and, importantly, NRAS^{MUT} melanoma PDXs. We evaluated (i) resistance-specific recurrence of amplicons harboring known and putative resistance-driver genes in CGRs and ecDNAs, (ii) overlap between the genomic coordinates of CGRs plus ecDNAs and those of chromothriptic regions, and (iii) inferred double-stranded DNA break (DSB) repair pathway(s) based on analysis of breakpoint-junctional sequences of CGR- and ecDNA-amplicons. Using human melanoma cell lines and PDXs, we tested the efficacy and dissected the mechanisms of blocking genomic instability, via targeting of NHEJ DSB repair, to prevent the evolution of acquired MAPKi resistance in both BRAF^{V600MUT} and NRAS^{MUT} melanoma.

### Methods

### Human Subjects

Patient characteristics related to clinical tissues are presented in Table 1. Patient-derived tissues were obtained with informed consents and approval by local institutional review boards.

TABLE 1
Clinical characteristics of patients who donated MAPKi-sensitive/-naive and acquired MAPKi-resistant melanoma

| | Patient/ | | | | Geno- | Biopsy | |
|---|---|---|---|---|---|---|---|
| Cohort | Sample ID | Sex | Age | Stage | type | sites | Treatment |
| Clinical | Pt1-Baseline | M | 65 | IV | BRAF_{V600MUT} | Cutaneous | Dabrafenib 150 mg (orally |
| | Pt1-DP1* | | | M1a | | Cutaneous | dosed twice a day) |
| | Pt1-DP3 | | | | | Cutaneous | |
| | Pt2-Baseline | M | 51 | IV | BRAF_{V600MUT} | Subcutaneous | Vemurafenib 960 mg (orally |
| | Pt2-DP1 | | | M1c | | Subcutaneous, | dosed twice a day) |
| | | | | | | flank | |
| | Pt3-Baseline | M | 61 | IV | BRAF_{V600MUT} | Subcutaneous, | Vemurafenib 960 mg (orally |
| | | | | M1c | | scalp | dosed twice a day) |
| | Pt3-DP1 | | | | | Subcutaneous | |
| | Pt4-Baseline | M | 37 | IV | BRAF_{V600MUT} | Cutaneous, | Dabrafenib 150 mg/trametinib |
| | | | | M1c | | left face | 2 mg (orally dosed twice a |
| | Pt4-DD-DP1A* | | | | | Cutaneous, | day) |
| | | | | | | left face | |
| | Pt4-DD-DP2B | | | | | Cutaneous, | |
| | | | | | | neck | |
| | Pt4-DD-DP3C | | | | | Cutaneous, | |
| | | | | | | neck | |
| | Pt5-Baseline | M | 65 | IV | BRAF_{V600MUT} | Subcutaneous, | Vemurafenib 960 mg (orally |
| | | | | M1c | | abdomen | dosed twice a day) |
| | Pt5-DP1 | | | | | Subcutaneous, | |
| | | | | | | flank | |
| | Pt5-DP2 | | | | | Subcutaneous | |
| | Pt6-Baseline | M | 74 | IIIc | BRAF_{V600MUT} | Cutaneous, | Vemurafenib 960 mg (orally |
| | | | | | | thigh | dosed twice a day) |
| | Pt6-DP2 | | | | | Cutaneous, | |
| | | | | | | thigh | |
| | Pt7-Baseline | M | 83 | IV | BRAF_{V600MUT} | Cutaneous, | Vemurafenib 960 mg (orally |
| | | | | M1c | | leg | dosed twice a day) |
| | Pt7-DP1 | | | | | Cutaneous, | |
| | | | | | | flank | |
| | Pt9-Baseline | M | 47 | IVa | BRAF_{V600MUT} | Cutaneous, | Vemurafenib |
| | | | | | | right thigh | 960 mg/GDC0973 |
| | Pt9-DP2 | | | | | Cutaneous, | (cobimetinib) 60 mg (orally |
| | | | | | | right thigh | dosed twice a day) |
| | Pt9-DD-DP1 | | | | | Cutaneous, | |
| | | | | | | right thigh | |
| | Pt9-DD-DP2 | | | | | Cutaneous, | |
| | | | | | | right thigh | |
| | Pt10-Baseline | M | 51 | IV | BRAF_{V600MUT} | Subcutaneous, | Vemu 960 mg (orally dosed |
| | | | | M1c | | scalp | twice a day) |
| | Pt10-DP1 | | | | | Subcutaneous, | |
| | | | | | | scalp | |
| | Pt11-Baseline | F | 41 | IV | BRAF_{V600MUT} | Breast | Vemu 960 mg (orally dosed |
| | Pt11-DP1 | | | M1c | | Lymph | twice a day) |
| | | | | | | node, | |
| | | | | | | axillary | |
| | Pt11-DP2 | | | | | Intramuscular, | |
| | | | | | | shoulder | |
| Rapid | RAM12.01-Sensitive | F | 29 | IV | BRAF_{V600MUT} | Parauterine | Vemurafenib |
| autopsy | | | | M1c | | lymph node | 960 mg/GDC0973 |
| melanoma | RAM12.01-Brain-DD-DP10 | | | | | Brain | (cobimetinib) 60 mg (orally |
| | RAM12.01-Brain-DD-DP3 | | | | | Brain | dosed twice a day) |
| | RAM12.01-Brain-DD-DP9 | | | | | Brain | |
| | RAM13003-Brain-DD-DP | M | 66 | IV | BRAF_{V600MUT} | Brain | Vemurafenib/dabrafenib- |
| | RAM13003-Lung-DD-DP | | | | | Lung | trametinib (orally dosed |
| | | | | | | | twice a day) |
| | RAM14006-Adrenal-DD-DP | M | 47 | IV | BRAF_{V600MUT} | Adrenal | Ipilimumab/Temozolomide/ |
| | RAM14006-Brain-DD-DP | | | | | Brain | Pembrolizumab/dabrafenib- |
| | RAM14006-LBD-DD-DP | | | | | Liver bile | trametinib |
| | | | | | | duct | |
| | RAM14006-Lymph1-DD-DP | | | | | Lymph | |
| | | | | | | node 1 | |
| | RAM14006-Lymph2-DD-DP | | | | | Lymph | |
| | | | | | | node 2 | |
| | RAM14006-Lung-DD-DP | | | | | Lung | |
| | RAM14006-Muscle-DD-DP | | | | | Muscle | |
| PDX | Mel_PDX16 | F | 76 | N/A | BRAF_{V600MUT} | Cutaneous, | Vemurafenib at 90 mg/kg/day + |
| | | | | | | right | Trametinib, 0.7 mg/Kg/day, |
| | | | | | | abdomen | various treatment conditions |
| | Mel_PDX27-V1 | M | 63 | N/A | BRAF_{V600MUT} | Left deltoid | Trametinib, 5 m g/Kg/day (to |
| | Mel_PDX27-R1 | | | | | mass | derive acquired resistance), |
| | Mel_PDX27-R2 | | | | | | also various treatment |
| | Mel_PDX27-R3 | | | | | | conditions |
| | Mel_PDX1-V1 | F | 85 | IV | NRAS_{MUT} | N/A | Trametinib 5 mg/kg/day (to |
| | Mel_PDX1-R1 | | | M1b | | | derive acquired resistance), |
| | Mel_PDX1-R2 | | | | | | also various treatment |
| | | | | | | | conditions |
| | Mel_PDX2-V1 | F | 80 | IV | NRAS_{MUT} | N/A | Trametinib, 5 mg/Kg/day (to |
| | Mel_PDX2-R2 | | | | | | derive acquired resistance), |
| | | | | | | | also various treatment |
| | | | | | | | conditions |
| | Mel_PDX3-V1 | F | N/A | N/A | NRAS_{MUT} | N/A | Trametinib, 5 mg/Kg/day (to |
| | Mel_PDX3-R4 | | | | | | derive acquired resistance) |
| | Mel_PDX3-R5 | | | | | | |
| | Mel_PDX4-V1 | M | 75 | N/A | NRAS_{MUT} | Cutaneous, | Trametinib, 5 mg/Kg/day (to |
| | Mel_PDX4-R2 | | | | | right wrist | derive acquired resistance), |
| | Mel_PDX4-R4 | | | | | | also various treatment |
| | Mel_PDX4-R5 | | | | | | conditions |
| | Mel_PDX6-V1 | F | 48 | IV | NRAS_{MUT} | Cutaneous, | Trametinib, 5 mg/Kg/day (to |
| | Mel_PDX-R3 | | | M1b | | right lower | derive acquired resistance) |
| | | | | | | leg, | |
| | | | | | | superior | |

*DP, Disease progression
*DD-DP, Double-drug disease progression

### Mice

NSG (NOD scid gamma) mice were obtained from the Radiation Oncology breeding colony at UCLA (Los Angeles, CA). Male or female mice were used at 4-6 weeks of age. All animal experiments were conducted according to the guidelines approved by the UCLA Animal Research Committee.

### PDX Models and In Vivo Treatments

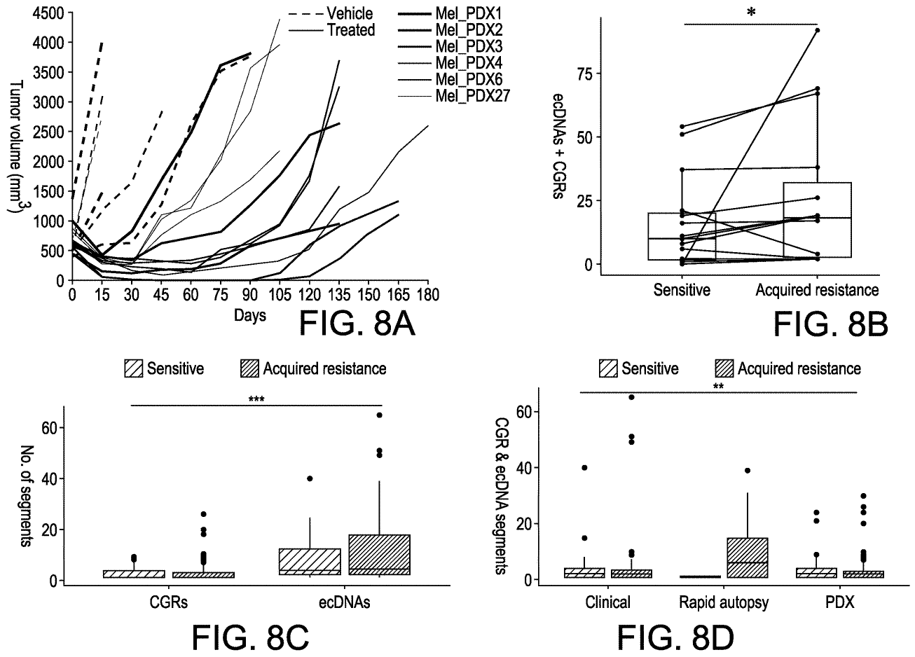

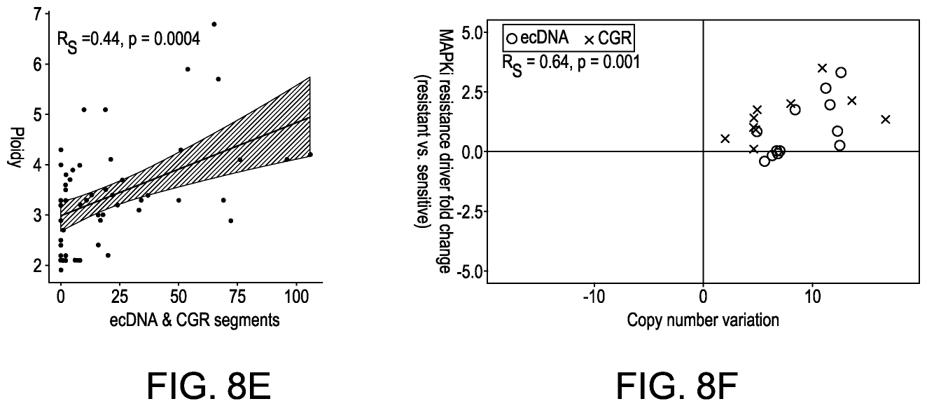

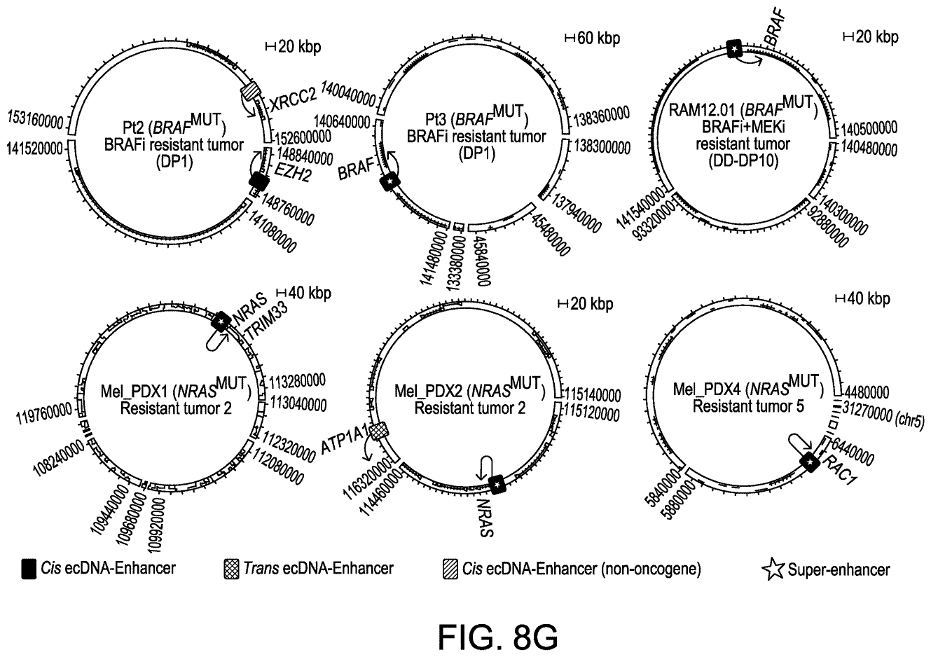

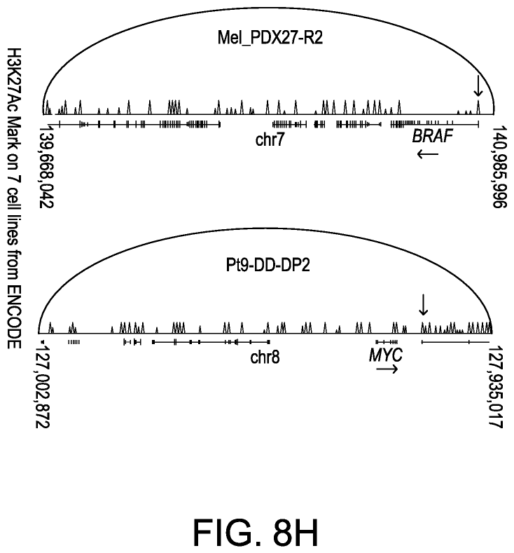

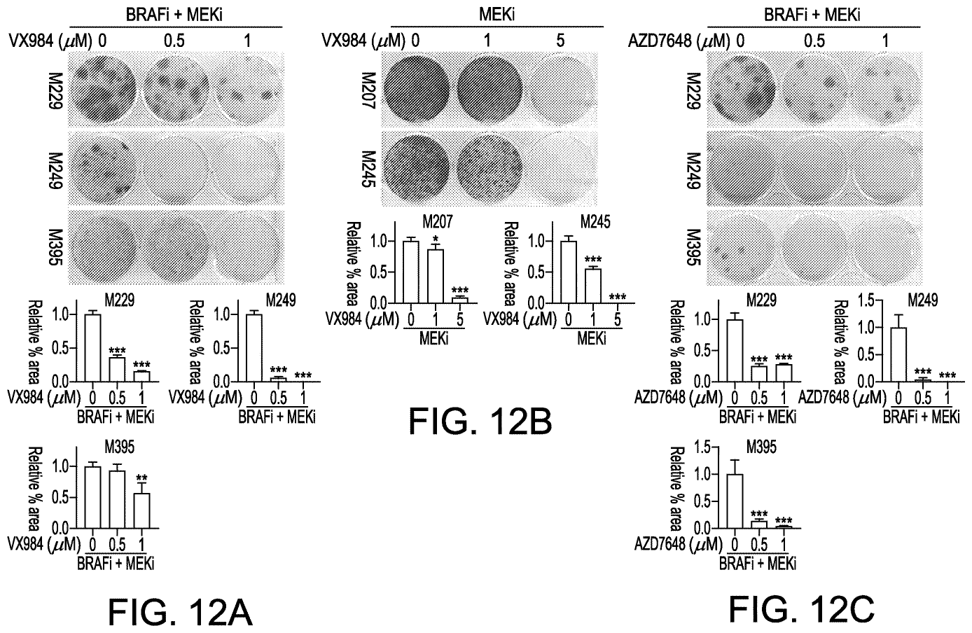

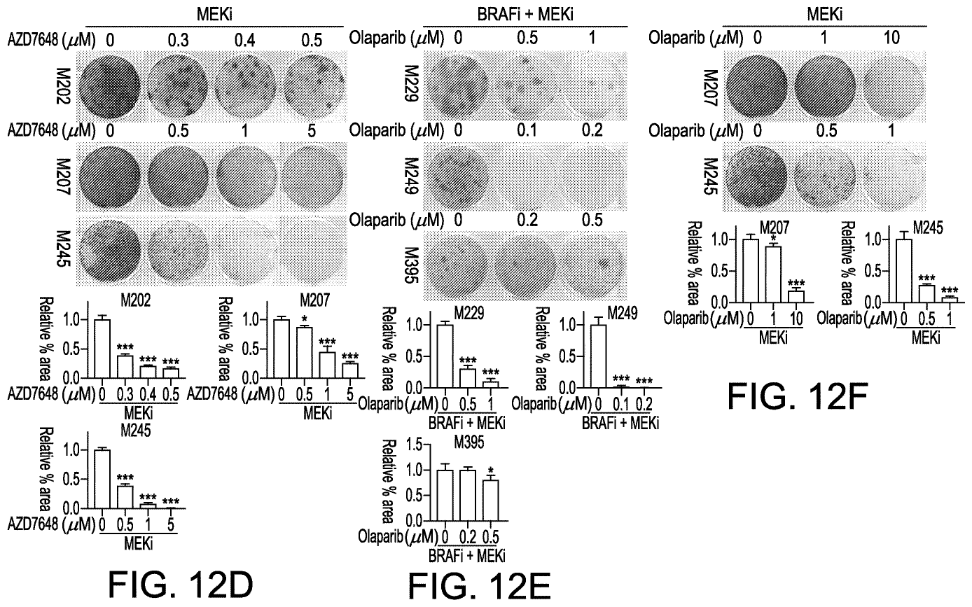

To develop PDX models, tumor fragments derived from metastatic melanoma, with approval by the local institutional review boards, were transplanted subcutaneously in sex-matched NSG mice (4-6 weeks old). One tumor fragment was implanted in each mouse. Tumors were measured with a caliper every 2 days, and tumor volumes were calculated using the formula (length×width²)/2. Tumors with volumes around 500 mm³ were randomly assigned into experimental groups. Special mouse diets were generated to reduce stress to animals by incorporating trametinib (LC Laboratories) into chow to achieve daily trametinib dosing at 3 or 5 mg/kg/day or combined vemurafenib (LC Laboratories) and trametinib dosing at 90 mg/kg/day and trametinib at 0.7 mg/kg/day, respectively (Test Diet, Richmond, IN, USA). DNA-PKi (NU7026, Selleckchem) was dissolved in saline (UCLA DLAM pharmacy) and administered intraperitoneally at 6, 8, or 10 mg/kg/day. We derived model- or patient-matched vehicle-treated tumors and acquired trametinib resistant tumors as plotted in FIG. 8A. We also derived model-matched vehicle-treated tumors and early on-treatment tumors as plotted in FIGS. 12C and 12D.

### Whole Genome Sequencing (WGS)

Genomic DNA (gDNA) and total RNA were extracted from frozen tumor tissue preserved in RNALater or snap-frozen tumor tissue using the QIAGEN AllPrep DNA/RNA Mini Kit and the Ambion mirVana miRNA Isolation Kit. Normal gDNA from PBMCs were extracted from fresh or frozen PBMCs using the QIAGEN FlexiGene DNA Kit. All gDNA were quantified using a NanoDrop (Thermo Fisher Scientific) and Qubit fluorometer using the dsDNA BR Assay (Life Technologies), then gDNA size and quality were tested using TapeStation (Agilent) to ensure gDNA libraries are prepared using equal gDNA input and presence of a high molecular weight band. Whole genome libraries were prepared using The Roche KAPA HyperPrep Kit. Briefly, after enzymatic fragmentation of gDNA, the libraries were constructed by end repairing and A-tailing the fragmented DNAs, ligation of adapters, and PCR amplification. After library construction, indexed libraries were quantified for equal molar pooling and paired-end sequenced with a read length of 2×150 bp on the Illumina NovaSeq 6000 S4 platform.

Whole-genome libraries were prepared for patient-matched normal tissues and tumors, as follows: (i) ten BRAF^{V600MUT} clinical patients (baseline tumors, n=10; resistant tumors, n=17), (ii) three BRAF^{MUT} RAM patients (sensitive tumor, n=1; resistant tumors, n=12), and (iii) six PDX models. PDX models included one BRAF^{MUT} PDX (vehicle-treated tumor, n=1; resistant tumors n=3) and five NRAS^{MUT} PDXs (vehicle-treated tumors, n=5; resistant tumors, n=9). For early on-treatment samples, we prepared whole-genome libraries from (i) one human BRAF^{V600MUT} (M229; DMSO, n=1; BRAFi+MEKi, n=1; BRAFi+MEKi+DNA-PKi, n=1) and one NRAS^{MUT} (M245; DMSO, n=1; MEKi, n=1; MEKi+DNA-PKi, n=1) cutaneous melanoma cell line and (ii) two BRAF^{V600MUT} (vehicle, n=6: BRAFi+MEKi, n=6; BRAFi+MEKi+DNA-PKi, n=9) and three NRAS^{MUT} (vehicle, n=6; MEKi, n=6; MEKi+DNA-PKi, n=7) cutaneous melanoma PDX models. Also, whole-genome libraries were prepared for vehicle-treated tumors and early on-treatment tumors from two NRAS^{MUT} PDX models. In total, paired-end sequencing was performed on 123 (104 tumors or cell lines, 19 patient-matched normal tissues) genomes using Illumina NovaSeq S4 with a read length of 2×150 bp and at a sequencing depth of 10-98× (median, 26×).

### WGS Data Analysis

### Genome Alignment, Copy Number, and Structural Variation Calling

WGS reads were mapped to GRCh38/hg38 human reference genome using BWA-MEM (53). Alignments were sorted and PCR duplicates were removed using Samtools (54). Copy number variations were called using two depth-of-coverage-based methods CNVkit (55) and ReadDepth (56). Default parameters were used for CNVkit. We used FDR of 0.05 and overdispersion of 1 for ReadDepth analysis. Structural variations reported by at least two SV detection methods: SvABA (57), TIDDIT (58), and DELLY (59) were considered. SVs in both DELLY and TIDDIT are determined by combining discordant read pairs and split-reads, while TIDDIT additionally employs depth-of-coverage signatures. SvABA utilizes discordant reads and genome-wide local assembly strategies for predicting SVs from the genome. For high coverage data (>15×), default parameters were considered for SvABA, TIDDIT, and DELLY. Parameter minimum number of points (−I) was set to 5 in TIDDIT for low coverage data.

Analysis of ecDNAs and CGRs

Reconstruction of focal gene amplifications, elucidation of ecDNA and CGRs was carried out using AmpliconArchitect (60). Briefly, AmpliconArchitect determines the list of potential intervals for each amplicon to be reconstructed and within each amplicon, copy numbers and structural variants are estimated using read depth and discordant read signatures. It then constructs breakpoint graph detailing sequence edges, breakpoint edges, and predicts copy counts of all edges. Simple cycles are then decomposed from the breakpoint graphs. Finally, AmpliconClassifier classifies the amplicons into ecDNAs, CGRs, and linear amplicons. Amplicons are classified as ecDNAs if the segment(s) form a head-to-tail structure, with size>10 kb, and copy number>4.5; as complex genomic rearrangements for noncircular amplicons containing DNA segments from different chromosomes or regions that are far apart (>1 Mb) on chromosomes; or as linear amplicons for linear amplifications. In our study, the initial set of copy number variation seed regions was inferred by ReadDepth and CNVKit. Structural variant view of amplicons and circle plots of simple cycles were generated using functions available in AmpliconArchitect.

We carried out pathway enrichment analysis of genes within ecDNAs and/or CGRs by using the Molecular Signature Data Base with pathways listed in GO, KEGG, Reactome, and Pathway Interaction Databases. For each tumor sample, we identified somatic SNVs using Strelka2 (61) with default parameters. Next, we estimated single base substitution (SBS) signatures in regions within and without ecDNAs and CGRs using the non-negative matrix factorization-based tool MutationalPatterns (62) with COSMIC SBS signatures V3.3 as reference. We carried out mutational signature analysis using SNVs in ecDNAs and/or CGR regions of (i) acquired MAPKi-resistant (mutations in patient-matched sensitive tumors subtracted from mutations in resistant tumors) and (ii) -sensitive genomes. To characterize whether SBS mutational signatures are preferentially detected within (versus outside of) ecDNAs and/or CGRs amplicons, we calculated the ratios of the proportions of signatures within ecDNA and/or CGR sequences and the proportion of signatures outside ecDNAs and/or CGR sequences and defined these ratios as SBS mutational signature enrichment score. We considered an enrichment score of one as the cut-off threshold. Scores>1 indicate SBS signatures enriched within ecDNAs and/or CGRs, while scores<1 indicate SBS signatures enriched in the background (non-ecDNA- and/or CGR-involved genomic regions). For clarity, only scores>1 and <1 were plotted.

We extracted enhancer elements and their connected genes from GeneHancer (version4-4, GeneCards). Genehancer is an integrated database of human enhancers and their inferred target genes, mined from four different genome-wide databases: the ENCODE, FANTOM, the VISTA enhancer browser and the Ensembl regulatory build. The enhancer-target genes associations were obtained from eQTLs, CHi-C, eRNA co-expression, transcription factor co-expression and gene-enhancer distance methods. Annotations of super enhancers were obtained from dbSUPER (63). Enhancers were first assessed for their presence on CGR- and ecDNA-amplicons and, if so, their connected oncogenes were searched for their presence within CGR-/ecDNA-amplicons (cis interaction) or elsewhere in the chromosome (trans interaction). Furthermore, we obtained H3K27 acetylation peaks from seven cell lines (GM12878, H1-hESC, HSMM, HUVEC, K562, NHEK and NHLF) listed in ENCODE. The peaks aligned to ecDNA regions in Pt9-DD-DP2 (MYC) and Mel_PDX27-R2 (BRAF) were obtained from the UCSC genome browser.

### Metaphase Chromosome Spread

Cells in metaphase were prepared by colcemid (Sigma) treatment at 10 μg/ml for several hours (depending on the growth rate). We then collected and washed with PBS single-cell suspensions, which were then treated with 0.075 M KCl (Gibco) for 15 min. We then fixed and washed cells with 3:1 methanol:acetic acid. The final cell pellet was resuspended with the fixative and dropped onto a humidified slide.

### DNA FISH

Formalin-fixed paraffin-embedded (FFPE) tissue samples were baked at 90° C. for 25 minutes in an oven and immersed in 100% xylene and then 100% ethanol, each for 10 minutes to deparaffinize tissues. Air-dried tumor tissues were pretreated in 90-95° C. 10 mM in citric acid buffer (pH 6.8, Thermo Fisher Scientific, 327162500) for 30 minutes and washed in 2×□SSC buffer (Invitrogen, 15557044) for 5 minutes. Then FFPE slides were digested in 37° C. pepsin solution (Thermo Fisher Scientific, J6167906) for 20-30 minutes, washed in 2×□SSC buffer for 5 minutes, and dehydrated in ascending ethanol series (70%, 85% and 100%), each for 2 minutes. For metaphase DNA FISH, fixed cells in interphase or metaphase on slides were dehydrated in ascending ethanol series (70%, 85% and 100%), each for 2 minutes. We used the following DNA-FISH probes: NRAS/CEN1 amplification probe (NRAS-CHR01-20-ORGR) targeting NRAS or centromeric region of chromosome 1; BRAF/CEN7 amplification probe (BRAF-CHR07-20-ORGR) targeting BRAF or centromeric region of chromosome 7; and RAF1/CEN3 amplification probe (RAF1-CHR03-20-ORGR) targeting RAF1 or centromeric region of chromosome 3, all from Empire Genomics. The probes were mixed with the provided hybridization buffer in 1:4 ratio and applied onto the tissues or cells. FFPE samples were then denatured at 75° C. in a slide moat for 7 minutes, while metaphase samples were denatured at 73° C. for 2 min. We then performed hybridization overnight at 37° C. in a humidified chamber. The samples were then washed in 73° C. 0.3% Igepal/0.4×SSC for 2 minutes, followed by another 2-minute wash with 0.1% Igepal/2×SSC at room temperature. Finally, the tissue samples were stained with ProLong™ Diamond Antifade Mountant with DAPI (Invitrogen, P36966) and covered by coverslips. Images were acquired on a Leica Confocal SP8-STED/FLIM/FCS microscope. For ecDNA quantification, we counted the number of specific gene foci in each nucleus by ImageJ (version, 1.52a). For HSR quantification, we counted the area of gene foci larger than 2 μm² to exclude ecDNA and non-HSR gene foci in each nucleus by ImageJ (version, 1.52a).

### RNA-Seq Analysis

Total RNAs were extracted from frozen tumor tissue preserved in RNALater of snap-frozen tumor tissues using the QIAGEN AllPrep DNA/RNA Mini Kit and the Ambion mirVana miRNA Isolation Kit. Total RNAs were quantified by the Qubit RNA High Sensitivity kit (Thermo Fisher Scientific) and/or using a NanoDrop (Thermo Fisher Scientific). RNA size and quality were measured using Agilent 2100 Bioanalyzer (Agilent Technologies). RNA libraries were constructed using the NuGen Universal Plus mRNA-Seq Kit. Briefly, after fragmentation of total RNA and double-stranded cDNA generation using a mixture of random and oligo(dT) primers, the RNA libraries were constructed by end-repairing, adapter-ligation, strand-selection, and PCR amplification. Libraries were quantified for equal molar pooling and paired-end sequenced with a read length of 2×50 bp on the Illumina NovaSeq 6000 S4 platform.

We mapped paired-end reads of patient-matched tumor samples from ten patients and six PDX models to the GRChr38 human reference genome using STAR aligner (64) with default parameters. Log 2 fold change values were calculated for each acquired resistant tumor compared to patient- or model-matched pretreatment tumor or vehicle-treated tumor, respectively. Genes with an absolute fold change>2 were considered significant. Gene counts were estimated using FeatureCounts (65) with GENCODE (version 38) annotations. Feature counts were then normalized using trimmed mean of M values (TMM) approach and log 2 transformed (66). Fold changes for each gene were estimated by computing the difference between the normalized values of acquired resistant tumors and MAPKi-sensitive/-naive transcriptomes.

### Analysis of Chromothripsis

To infer chromothriptic events in the genomes, we applied ShatterSeek (12), an in silico scoring algorithm based on the clusters of interleaved SVs, CN oscillations, and inter-chromosomal SVs. Copy number and structural variants described above were considered as input for ShatterSeek. High confidence chromothriptic events were selected based on the statistical criteria recommended by the authors. Briefly, a high confidence chromothriptic event is characterized with (i) a cluster of SVs (>6 DUP/DEL/h2hINV/t2tINV), (ii) oscillating CN between two states (>7 CN events), (iii) chromosomal enrichment and distribution of DNA breakpoints (p<0.05), (iv) randomness of fragment joins (p>0.05) and/or (v) inter-chromosomal rearrangements between multiple chromosomes. Somatic SNVs spanning chromothripsis regions were identified using Strelka2 (61) with default parameters and defined as chromothripsis-associated SNVs. Regions with and without chromothripsis were extracted and tumor mutational burdens were computed within these regions for each tumor sample. Ratios between tumor mutational burdens within and outside of chromothriptic regions were calculated. Mutational signature analysis was carried out using SNVs in chromothriptic regions of (i) acquired MAPKi-resistant (mutations in patient-matched sensitive tumors subtracted from mutations in resistant tumors) and (ii) -sensitive genomes. Mutational (SBS, DBS, and ID) signatures for each sample were predicted using non-negative matrix factorization-based tool MutationalPatterns (62) and the extracted signatures were compared with the COSMIC-SBS, DBS, and ID signature database.

### Junctional Sequence Analysis of Amplicon Breakpoints

Sequences of CGRs and ecDNAs identified in all the genomes were analyzed. Breakpoints of amplicons harboring key MAPKi resistance genes, BRAF, NRAS, HRAS, and EGFR were extracted, and sequences spanning these junctions were further analyzed. Homologous sequences and insertions for the breakpoint junctions were obtained from SvABA, and a possible mechanism for DNA DSB repair was inferred. First, HRR was identified with large homologous sequences and insertions (2:8 bp). Second, MMEJ or alt-NHEJ was identified with breakpoints comprising homologous sequences (2-8 bp). Third, NHEJ was identified when neither of the sequence types described above was identified in the vicinity of the breakpoints.

### Cell Lines and Inhibitor Treatments

All cell lines were routinely tested for mycoplasma and profiled and identified by RNA-seq and the GenePrint 10 system (Promega) at periodic intervals during the course of this study. All M series cell lines were established from patient-derived tumors at the University of California, Los Angeles (UCLA) with IRB approval. All M cell lines with acquired MAPKi resistance were derived in the Lo Laboratory and published previously (3,5,8,9,11,22,40,67). We maintained H538 (ATCC) in RPMI-1640 (Gibco) with 10% heat-inactivated FBS (Omega Scientific, FB-02) and 2 mM glutamine in a humidified, 5% CO₂ incubator at 37° C.; all other cell lines were maintained in high glucose DMEM (Omega Scientific, DM-22) with 10% heat-inactivated FBS (Omega Scientific, FB-02) and 2 mM glutamine in a humidified, 5% CO₂ incubator at 37° C. We obtained inhibitors from the following sources: PLX4032 (Plexxikon), AZD6244 (Selleck Chemicals), trametinib (LC Laboratories), NU7026 (Abcam, ab120970), ABT888 (Enzo, ALX-270-444-M005), VX984 (MCE, HY-19939S), AZD7648 (TargetMol, T7122), olaparib (LC Laboratories, 763113-22-0), MRTX849 (Selleckchem, S8884), AMG510 (Selleckchem, S8830), and BGB-283 (BeiGene, via a Material Transfer Agreement with UCLA). All inhibitors were dissolved in DMSO and stored at −20° C.

Lentivirus Production, Transduction, and shRNA Sequences

shRNAs for PRKDC and vector control (pGIPZ) were obtained from Robert Damoiseaux, Ph.D. (Molecular Screening Shared Resource, UCLA), shRNAs for LIG4 from Sigma, and packaged into lentiviral particles for infection. The lentiviral viruses were generated by transfection of the constructs together with pMD2.G, pRSV-Rev and pMDLg/pRRE into HEK-293T cells using calcium phosphate. Fourteen hours after transfection, media were replaced with pre-heated fresh media. Virus particles were harvested 24 and 48 hours later and filtered by 0.45 μm filter unit (Millipore). Cells were transduced with recombinant lentivirus with 10 μg/mL polybrene (Millipore, TR-1003-G) for 48 hours and then selected by puromycin (Sigma, P8833) for one week. shRNA targeting sequences are as follows:

Human PRKDC sh1:
(SEQ ID NO: 1)
CAGTGAAAGTCTGAATCAT
Human PRKDC sh2:
(SEQ ID NO: 2)
GTCATGGATTCAAGAAATA
Human PRKDC sh3:
(SEQ ID NO: 3)
GAGCTTACATGCTAATGTA
Human PRKDC sh4:
(SEQ ID NO: 4)
GGTCATGGATTCAAGAAAT
Human LIG4 sh1:
(SEQ ID NO: 5)
TTCGACGCCACACCGTTTATT
Human LIG4 sh2:
(SEQ ID NO: 6)
TATGTCAGTGGACTAATGGAT

### Cell Growth Assays

For clonogenic assay, cells were plated at indicated cell densities in six-well plates and treated with inhibitor(s) the next day. Inhibitor(s) and media were replenished every 2 days for the number of days indicated. Colonies were fixed in 4% paraformaldehyde, followed by staining with 0.1% crystal violet. For MTT assay, cells were plated at 2,000 cells per well in 96-well plates; acquired resistant sub-lines were seeded at 4,000 cells per well and treated with graded concentrations of MAPKi the next day; and media were replenished every 2-3 days. 100 μL Methylthiazolyldiphenyl-tetrazolium bromide (MTT) solution (0.5 mg/ml, Sigma, M5655) was added to each well and incubated at 37° C. for 2 h for MTT formazan formation. Formazan was then dissolved in 100 μL DMSO, and the absorbance values were measured using an ELISA reader (SpectraMax Plus 384) at the wavelength of 570 nm, blanked with DMSO solution. Experiments were performed with four replicates.

### Western Blots

Cells were lysed in RIPA buffer (Thermo Fisher Scientific) with protease inhibitor cocktail (Thermo Fisher Scientific) and phosphatase inhibitor cocktails (Thermo Fisher Scientific) for Western blotting. BCA protein assay (Thermo Fisher Scientific, P123227) was used to determine the protein concentration. Antibodies used in Western blot are as follows: TUBULIN (CST, 2144S), PRKDC (CST, 38168S), and LIG4 (CST, 14649S).

### Immunofluorescence Analysis

Tumor tissues were fixed in formalin followed by paraffin-embedding. After deparaffinization and rehydration, tissue sections were antigen-retrieved by heat. Permeabilization and blocking were followed by overnight incubation with primary antibodies [p-ERK1/2 (Cell Signaling Technology, 4370), p-DNA-PKcs (Abcam, ab124918), p-H2AX (Cell Signaling Technology, 9718)]. Immunofluorescence was performed with Alexa Fluor-conjugated secondary antibodies (Life Technologies, A-21429). Nuclei were counterstained by DAPI. Signals were captured with a Zeiss microscope (AXIO Imager A1) mounted with a charge-coupled device camera (Retiga EXi QImaging), and images were captured by Image-Pro plus 6.0.

### Statistical Methods

Statistical analysis for genomic data were conducted in R.4.02, Python 3.8.0 and Python 2.7.17. Statistical analyses, described for specific experiments, for cell line- or PDX tumor-based assays were performed using GraphPad Prism (San Diego, CA).

### Data Availability

The BAM files of WGS data are deposited in the European Genome-phenome Archive (https://www.ebi.ac.uk/ega/) with the accession number EGAS00001006874.

### Results

### Advanced Cutaneous Melanoma Cohorts and Whole Genome Sequencing (WGS) Data Characteristics

To date, our mutational survey of acquired MAPKi-resistance (in patients with cutaneous BRAF^{600MUT} melanoma and treated with BRAFi or BRAFi+MEKi therapy) has been limited to whole exome analysis (5,7-9,21). Thus, we assembled three cohorts of tissues (Table 1) for WGS-based analysis of SVs. The first cohort consisted of patient-matched normal tissues, BRAF^{V600MUT} melanoma tumors before MAPKi therapy and, after initial responses, at disease progression (n=10 normal tissues; n=10 pretreatment tumors; n=17 acquired-resistant tumors; n=10 patients). The second cohort consisted of rapid autopsy (cutaneous) melanoma (RAM) tissues (n=3 normal tissues; n=1 sensitive BRAF^{V600MUT} tumor; n=12 acquired-resistant tumors; n=3 deceased subjects who were treated with MAPKi; n=6 metastatic organ sites). The third cohort consisted of cutaneous BRAF^{V600MUT} or NRAS^{MUT} PDX tumors. To study acquired MAPKi-resistance at the whole-genome level, we subjected PDXs (n=6 models; 1 BRAF^{MUT} and 5 NRAS^{MUT} models) to MAPKi therapy in NOD-scid IL2R gamma null (NSG) mice at doses sufficient to elicit tumor regression, and then generated acquired MAPKi-resistant tumors (FIG. 8A) (11,22). In total, we used vehicle-treated tumors (n=6), acquired-resistant tumors (n=12), and patient-matched normal tissues (n=6) to generate WGS data.

Acquired Resistance-Associated ecDNAs and CGRs Outnumber Those Detected in MAPKi-Naive Melanoma

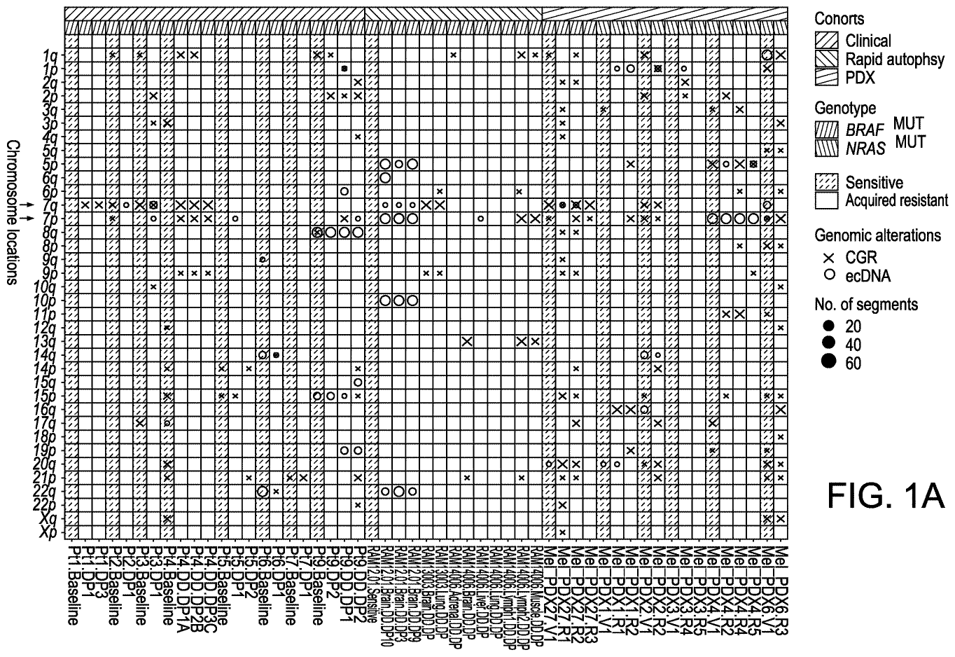

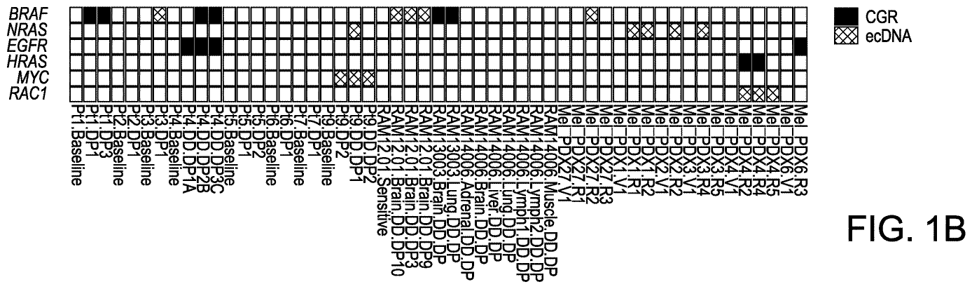

We identified and contrasted the ecDNA- and CGR-amplicons selected without and with MAPKi therapy. Considering all three cohorts (total sensitive tumors, n=17; total acquired-resistant tumors, n=41; total patients, n=19), we detected ecDNAs and CGRs at a highly recurrent rate: ~70% of sensitive and ~77% of acquired-resistant genomes harbored these amplicons. Reconstruction of ecDNA- and CGR-derived, high copy-number amplicons was carried out from WGS data of 58 BRAF^{V600MUT} and NRAS^{MUT} MAPKi-sensitive/resistant tumors. With all three cohorts combined, we observed a greater number of ecDNAs and CGRs in acquired-resistant (versus sensitive) genomes (acquired-resistant: n=941 total, n=31 average per tumor; sensitive: n=241 total, n=16 average per tumor; unpaired Student's t-test, p=0.09) (FIG. 1). In a patient-matched analysis of the clinical cohort, 12 of pairwise comparisons showed a higher number of ecDNA- and CGR-amplicons in acquired-resistant genomes (Wilcoxon test, p=0.033) (FIG. 8B). Overall, chr7p (26 of 53 genomes) and chr7q (23 of 53 genomes) emerged as hotspots (fragile sites) for CGR- and ecDNA-amplicon formation (FIG. 1). In addition, we observed resistance-associated CGRs in chr9p (8 of 41 resistant tumors across all three clinical-autopsy-PDX cohorts) and lower-frequency resistance-associated CGRs (in 2q, 4q, 6p, 8q, 9q, 10q, 18p, 22p, Xp) and ecDNAs (in 6q, 10p). In the total cohort, regardless of whether the tumor was sensitive or resistant to MAPKi, the number of rearranged segments was higher within ecDNAs (versus CGRs) (p=3.5e-08, unpaired Student's t-test) (FIG. 8C), suggesting ecDNAs as highly dynamic foci of genomic instability. Furthermore, the mean number of rearranged segments was highest in the RAM (~10 segments per tumor) followed by the clinical (~5 segments per tumor) and PDX (~4 segments per tumor) cohorts (FIG. 8D; Kruskal-Wallis rank test, p=0.01). This may reflect the highly advanced evolutionary state of terminal metastatic melanoma in the RAM cohort. Polyploidy has been associated with the onset of chromothripsis (12). Accordingly, we observed higher numbers of CGRs and ecDNAs in genomes with higher ploidy (FIG. 8E).

CGR- and ecDNA-Amplicons Drive MAPKi Resistance

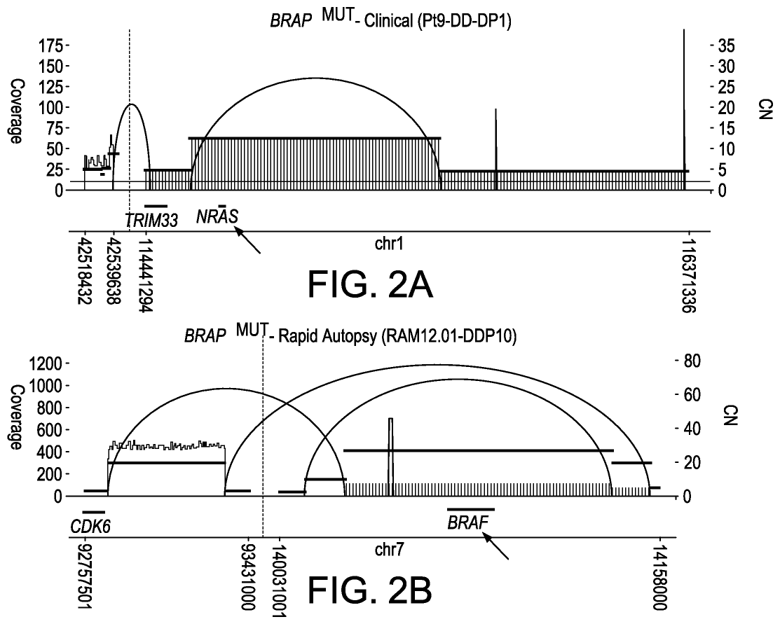

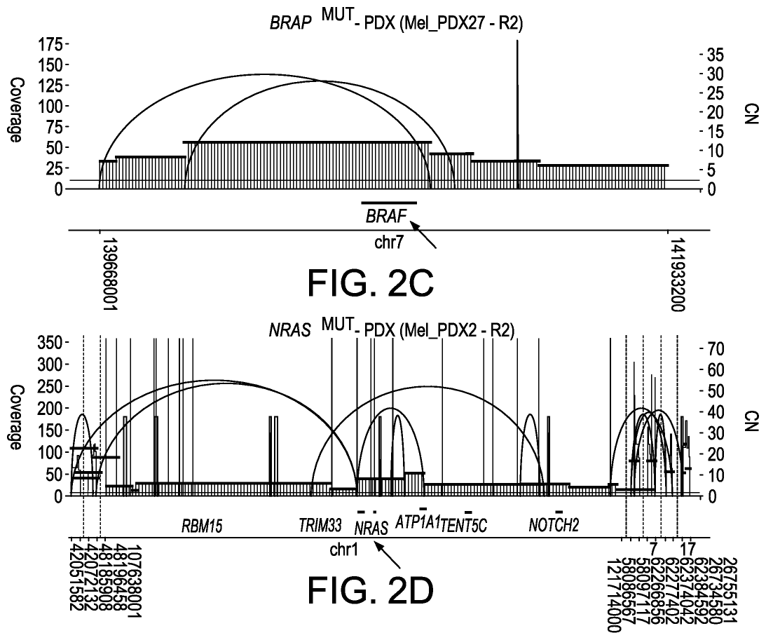

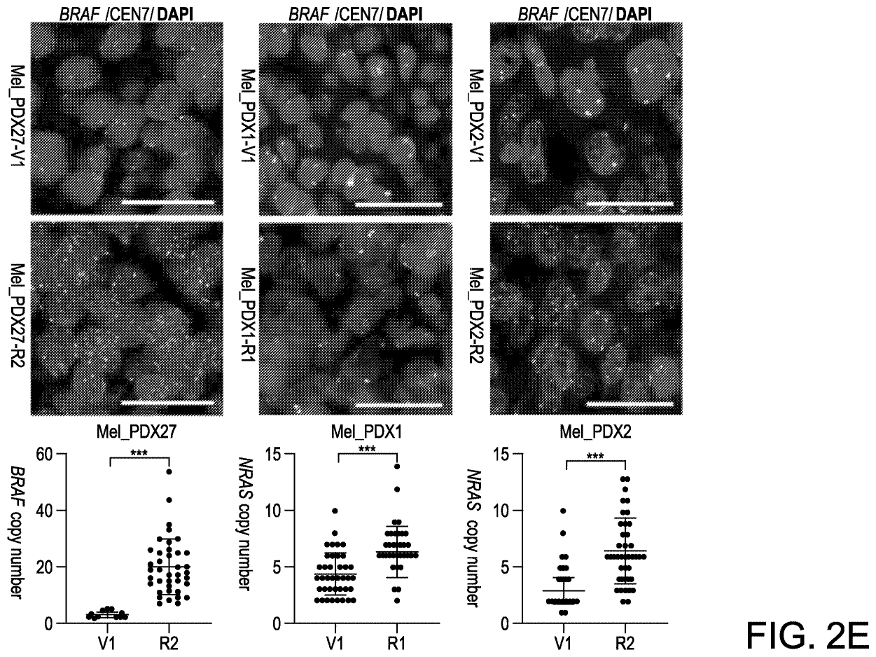

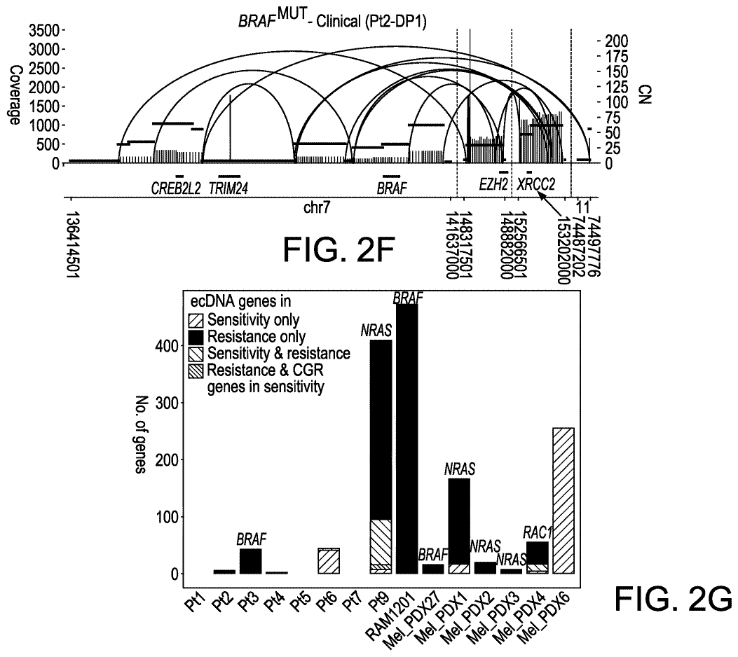

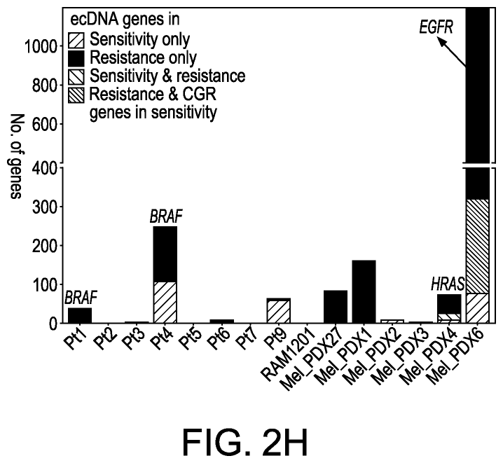

Analysis of genomic amplicons due to intrachromosomal CGRs and ecDNAs uncovered a significant (unpaired Student's t-test, p=0.0002) association between acquired-resistant tumors and CGRs and/or ecDNAs harboring bona fide MAPKi-resistance genes. BRAF (Copy number of CN range 4.5-27), NRAS (CN 5-13), HRAS (CN 13-16), MYC (CN 12-15) and EGFR (CN 4.6-5) (FIG. 1 and FIG. 2A to 2D), when amplified, are known to drive acquired MAPKi-resistance and MAPK pathway-reactivation (5,7-9,11). RACI (CN 6-7) in an ecDNA-amplicon was specifically observed in acquired resistance in NRAS^{MUT} melanoma PDXs, which suggests regulation of MLK3-CRAF by RAC1 (23). Moreover, we validated a recurrent ecDNA by direct isolation and high-depth sequencing (NRAS amplification, CN 13, Pt9-DD-DP1; FIG. 2A) using a new approach referred to as CRISPR-CATCH (24). This alternative technique confirmed the circularized junctions of a 890 kb, driver ecDNA within this acquired-resistant clinical tumor sample. In addition, we used DNA-FISH to cross-validate the resistance-specific presence and the CNs of BRAF and NRAS amplicons (FIG. 2E). In total, among the 17 of 19 patients included in this study whose tumors harbored ecDNAs and/or CGRs, MAPK-reactivation genes were amplified via ecDNAs and/or CGRs specifically in acquired-resistant tumors (but not in any MAPKi-sensitive/naive tumor) at a high frequency (60% or 23 of 38 resistant tumors). Expectedly, MAPKi driver gene amplification often co-occurred with other resistance-specific CNAs and somatic mutations reported earlier (7-9). Using RNA-seq data available from a subset of the patient-matched tumors, CN alterations (resistance versus sensitive) of MAPK-reactivation genes were highly correlated with corresponding mRNA level changes (Spearman's correlation rₛ=0.64, p=0.001) (FIG. 8F). In a minority of patients (Pt5, ecDNA⁺CGR⁺; Pt11, ecDNA⁻CGR⁻), we observed lower-level but linear amplifications of BRAF (CN 3-4) specifically in acquired-resistant tumors. The BRAF^{V600MUT} melanoma tumors from both patients were treated with only BRAFi (not BRAFi+MEKi), suggesting that low-level linear amplifications suffice in driving acquired resistance to BRAFi monotherapy.

NHEJ and homologous recombination repair (HRR) of DNA DSBs are thought to be critical for CGR and ecDNA generation after chromothripsis (20). Intriguingly, we observed that XRCC2 (RAD51-like; CN, 49), a key HRR gene, and XRCC6 (KU70; CN, 14-15), a key NHEJ gene, were amplified specifically in acquired MAPKi-resistant melanoma as ecDNAs (FIG. 2F). Moreover, acquired MAPKi-resistance was associated with ecDNA- or CGR-amplicons spanning other DSB repair genes in the NHEJ (TR/M33 CN, 7-11; PAX/P1 CN, 3) and HRR (SSBP1 CN, 5; BRCA2 CN, 6; RFC3 CN, 6: TR/M33 CN, 7-11; SYCP1 CN, 7-9, TR/M24 CN, 5-11) pathways. Thus, acquired MAPKi-resistance is specifically associated with CGR- and ecDNA-amplicons in MAPK-reactivation and DSB repair genes, which suggests functional interplay as co-drivers of resistance.

Resistance-specific genes amplified by ecDNAs and/or CGRs may contribute functionally to the resistant phenotype. Therefore, we identified the genes and their numbers in ecDNA (FIG. 2G) or CGR (FIG. 2H) amplicons specifically associated with sensitivity, resistance, or both sensitivity plus resistance. Importantly, genes amplified by either ecDNAs or CGRs specifically in resistance highly outnumbered those specific to sensitive tumors, which indicates that gene amplification by ecDNAs and CGRs contributes to disease progression on MAPKi therapy. Moreover, genes amplified by either ecDNAs or CGRs in both sensitive plus resistant tumors constituted a very small fraction, which is consistent with the notion that gene amplification by ecDNAs and CGRs contributes to disease progression. We rarely detected genes that overlapped in patient-matched sensitivity-associated CGRs and resistance-associated ecDNAs (and vice versa) (FIGS. 2G and 2H). To explore the functional contributions of ecDNA- and CGR-amplified genes to the acquired MAPKi-resistant phenotype, we performed pathway enrichment analysis using genes amplified by ecDNAs and/or CGRs specifically detected in eight evaluable (see Methods) cases of resistance (FIG. 2I). This analysis nominated alterations in MAPK (in Pt3, PDX27), PI3K-AKT (RAM12.01, PDX4), immune (Pt4, RAM12.01, PDX4), GPCR (Pt4, PDX6), cellular differentiation/morphogenesis (Pt3, Pt4, Pt9, PDX4, PDX6), and metabolism (Pt9, RAM12.01, PDX27, PDX1, PDX6) pathways as resistance phenotypes driven by ecDNA-/CGR-amplicons (Table 2).

TABLE 2
MAPK-reactivation amplicons in acquired resistance,
copy numbers, and amplicon sub-types

| | | Copy number | Amplification | |
|---|---|---|---|---|
| Sample | Gene | (Absolute value) | type | Cohort |
| Pt1-DP1 | BRAF | 8.0 | CGR | Clinical |
| Pt1-DP3 | BRAF | 10.9 | CGR | Clinical |
| Pt3-DP1 | BRAF | 11.6 | ecDNA | Clinical |
| Pt4-DD-DP2B | BRAF | 4.6 | CGR | Clinical |
| Pt4-DD-DP3C | BRAF | 4.8 | CGR | Clinical |
| Pt5-DP1 | BRAF | 3.0 | Low-linear | Clinical |
| Pt5-DP2 | BRAF | 3.0 | Low-linear | Clinical |
| Pt11-DP1 | BRAF | 3.9 | Low-linear | Clinical |
| RAM12.01-Brain-DD-DP3 | BRAF | 9.5 | ecDNA | RAM |
| RAM12.01-Brain-DD-DP9 | BRAF | 7.3 | ecDNA | RAM |
| RAM12.01-Brain-DD-DP10 | BRAF | 26.7 | ecDNA | RAM |
| RAM13003-Brain-DD-DP | BRAF | 4.5 | CGR | RAM |
| RAM13003-Lung-DD-DP | BRAF | 9.4 | CGR | RAM |
| RAM14006-Adrenal-DD-DP | BRAF | 3.0 | Low-linear | RAM |
| RAM14006-Brain-DD-DP | BRAF | 3.0 | Low-linear | RAM |
| RAM14006-Liver-DD-DP | BRAF | 4.0 | Low-linear | RAM |
| RAM14006-Lymph2-DD-DP | BRAF | 3.2 | Low-linear | RAM |
| RAM14006-Muscle-DD-DP | BRAF | 3.0 | Low-linear | RAM |
| Mel_PDX27-R1 | BRAF | 9.7 | Linear | PDX |
| Mel_PDX27-R2 | BRAF | 11.2 | ecDNA | PDX |
| Mel_PDX27-R3 | BRAF | 16.2 | Linear-invalid | PDX |
| Pt9-DD-DP1 | NRAS | 12.6 | ecDNA | Clinical |
| Mel_PDX1-R1 | NRAS | 7.0 | ecDNA | PDX |
| Mel_PDX1-R2 | NRAS | 6.7 | ecDNA | PDX |
| Mel_PDX2-R2 | NRAS | 8.4 | ecDNA | PDX |
| Mel_PDX3-R4 | NRAS | 4.9 | ecDNA | PDX |
| Pt4-DD-DP1A | EGFR | 4.9 | CGR | Clinical |
| Pt4-DD-DP2B | EGFR | 4.6 | CGR | Clinical |
| Pt4-DD-DP3C | EGFR | 4.6 | CGR | Clinical |
| Mel_PDX6-R3 | EGFR | 4.5 | CGR | PDX |
| Mel_PDX6-V1 | EGFR | 5.3 | Linear-invalid | PDX |
| RAM14006-Adrenal-DD-DP | EGFR | 3.0 | Low-linear | RAM |
| RAM14006-Brain-DD-DP | EGFR | 3.0 | Low-linear | RAM |
| RAM14006-Liver-DD-DP | EGFR | 4.0 | Low-linear | RAM |
| RAM14006-Lymph1-DD-DP | EGFR | 2.8 | Low-linear | RAM |
| RAM14006-Lymph2-DD-DP | EGFR | 3.5 | Low-linear | RAM |
| RAM14006-Muscle-DD-DP | EGFR | 3.0 | Low-linear | RAM |
| RAM14006-Lung-DD-DP | EGFR | 3.1 | Low-linear | RAM |
| Mel_PDX4-R2 | HRAS | 13.6 | CGR | PDX |
| Mel_PDX4-R4 | HRAS | 16.7 | CGR | PDX |
| Pt9-DP2 | MYC | 15.2 | ecDNA | Clinical |
| Pt9-DD-DP1 | MYC | 12.5 | ecDNA | Clinical |
| Pt9-DD-DP2 | MYC | 12.3 | ecDNA | Clinical |
| Pt9-Baseline | MYC | 10.7 | Linear-invalid | Clinical |
| Mel_PDX6-V1 | MYC | 5.4 | Linear-invalid | Clinical |
| Mel_PDX4-R2 | RAC1 | 5.6 | ecDNA | PDX |
| Mel_PDX4-R4 | RAC1 | 6.3 | ecDNA | PDX |
| Mel_PDX4-R5 | RAC1 | 6.8 | ecDNA | PDX |
| Mel_PDX4-V1 | RAC1 | 8.2 | Linear-invalid | PDX |
| Mel_PDX6-V1 | RAC1 | 5.3 | Linear-invalid | PDX |

Low-linear: Amplification filtered out by AmpliconArchitect due to copy gain cut-off of 4.5
Linear-invalid: Amplification with not enough support to call as ecDNA, CGR or linear by AmpliconArchitect

Enhancers within ecDNAs have been suggested to influence oncogene expression by either co-amplifying with target oncogenes within the ecDNAs (i.e., cis interaction) or regulating intrachromosomal genes (i.e., trans interaction) (16,25,26). Thus, we annotated CGR- and ecDNA-amplicons in both sensitive and acquired-resistant genomes with enhancers listed in GeneHancer (27). Notably, MAPKi-resistance genes (e.g., BRAF, NRAS, HRAS, EGFR) and known oncogenes (e.g., EZH2, CREB3L2, CARD11, EP300) were co-amplified with their corresponding enhancers within CGRs and ecDNAs of the MAPKi-resistant genomes (FIG. 8G). Enhancers associated with DNA DSB repair genes, such as XRCC2, XRCC6, and RAD21, were also observed within close proximity (−0.5 kb to +1.1 kb) (FIG. 8G). Furthermore, 10 of 11 and 4 of 5 acquired-resistant genomes with CGR- or ecDNA-associated BRAF and NRAS amplifications, respectively, harbored co-amplification of super-enhancers, which suggests that genomic plasticity may facilitate epigenomic plasticity and alter gene expression on a large-scale in MAPKi-resistant tumors. In further support of ecDNAs playing a role in epigenomic plasticity, we observed that H3K27 acetylated genomic regions (extracted from ENCODE data) were rearranged to the proximity (e.g., c-MYC) and co-amplified (e.g., BRAF) with ecDNA genes in acquired resistance (FIG. 8H). Enhancers docking sites within ecDNA-amplicons may also influence the differentiation state(s) of MAPKi-resistant tumors in trans, by acting on intrachromosomal genes such as HOXA9, HOXA11, HOXA13, LIFR., etc. (RAM12.01-Brain-DD-DP10, RAM12.01-Brain-DD-DP3, RAM12.01-Brain-DD-DP9) and acting as mobile regulatory elements for genes such as SMO (Pt3-DP1) and ATP1A1 (Mel_PDX2-R2) (FIG. 8G). Thus, CGR- and ecDNA-amplicons co-amplify MAPKi-resistance driver genes with their enhancers and may harbor additional enhancer or super-enhancer activities in cis or trans that concomitantly reprogram the transcriptome.

MAPKi-Naive and Acquired Resistant Melanoma Harbor Pervasive Chromothriptic Genomic Spans that Overlap ecDNA and CGR Sequences

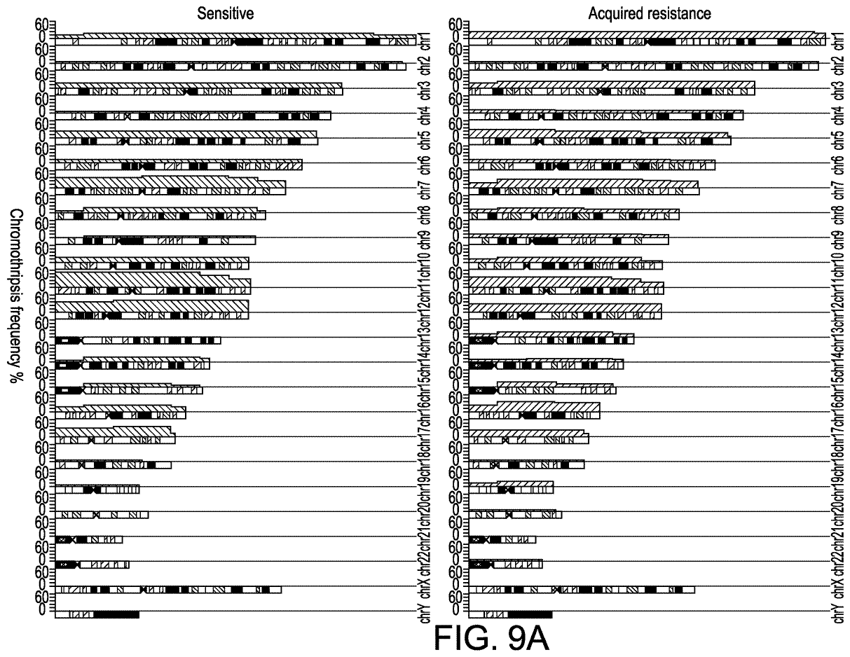

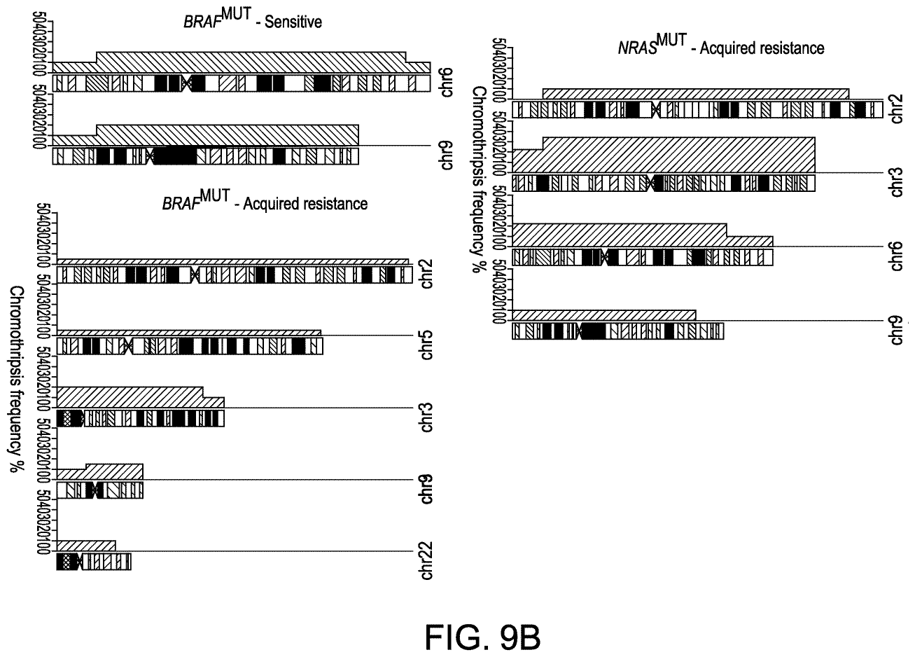

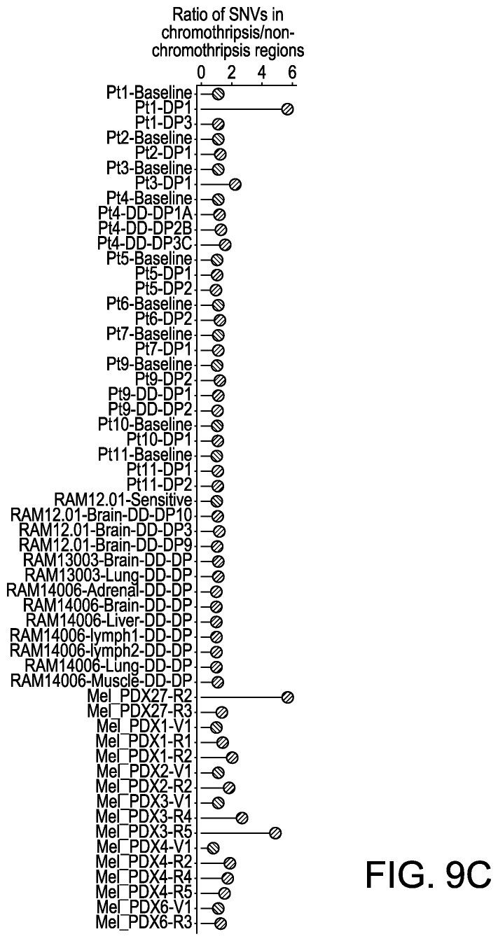

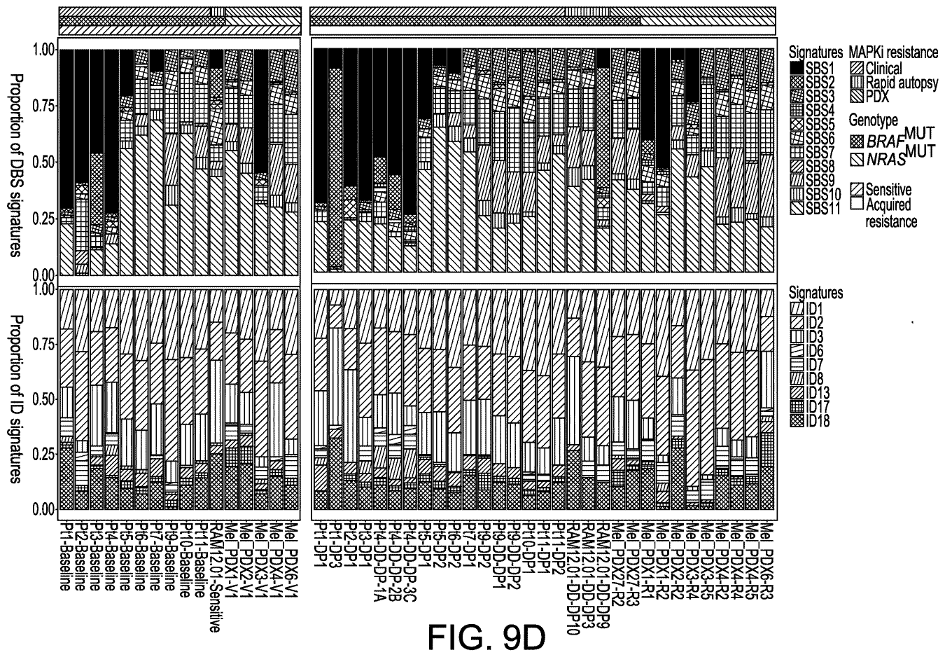

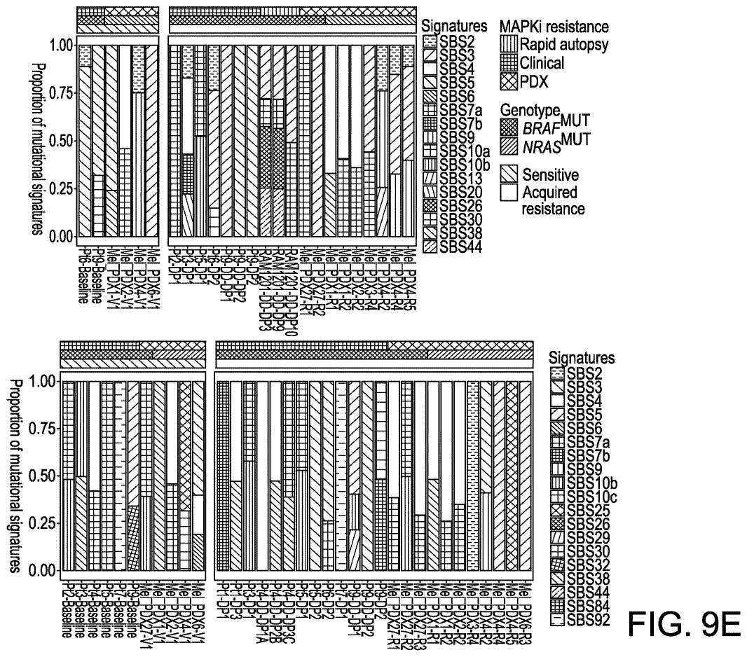

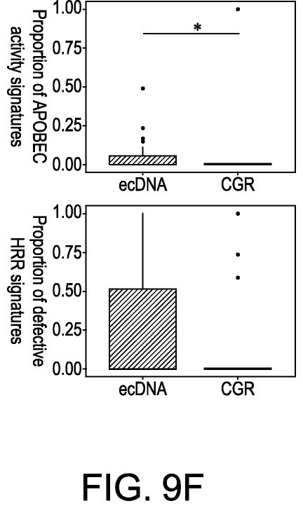

Chromothripsis, defined as a mutational phenomenon leading to extensive genomic rearrangements and extensive copy number oscillations, drives cancer initiation and progression (12). Prior studies have co-localized chromothriptic regions with ecDNAs (20,28-30), and a recent study suggested chromothripsis as a pathway for ecDNA formation in general (30) and specifically in ecDNA-driven methotrexate resistance (20). To evaluate the specific contributions of chromothripsis pre- and post-evolution of MAPKi resistance, we analyzed WGS data from 58 patient-matched, BRAF^{V600MUT} or NRAS^{MUT} MAPKi-sensitive/naive and acquired-resistant tumors (along with 19 patient-matched normal gDNAs). We observed a high prevalence of chromothripsis (high-confidence calls based on recently published criteria (12)) in MAPKi-sensitive (16 of 17; mean total span per genome, 464 Mb) and acquired-resistant (40 of 41; mean total span per genome, 476 Mb) melanoma. The frequencies and chromosome distributions of chromothripsis overlapped but were clearly distinct between the sensitive and acquired-resistant genomes (FIGS. 9A and 9B). Chromothriptic genomic spans in MAPKi-sensitive/naive and acquired MAPKi-resistant cutaneous melanoma overlap 76% and 36%, respectively, with those reported earlier for a subset of TCGA SKCM (12), suggesting that MAPKi selects for chromothripsis in distinct and overlapping genomic regions. Interestingly, the single MAPKi-sensitive melanoma without chromothripsis (Mel_PDX27-V1) still gave rise to two of three acquired-resistant tumors with chromothripsis. The numbers and span sizes of chromothriptic events in our melanoma cohorts are higher than those reported for melanoma in the PCAWG study (mean total span per genome, 120 Mb) (12). These differences are likely due to further evolutionary selection as a result of advancing disease and therapy resistance.

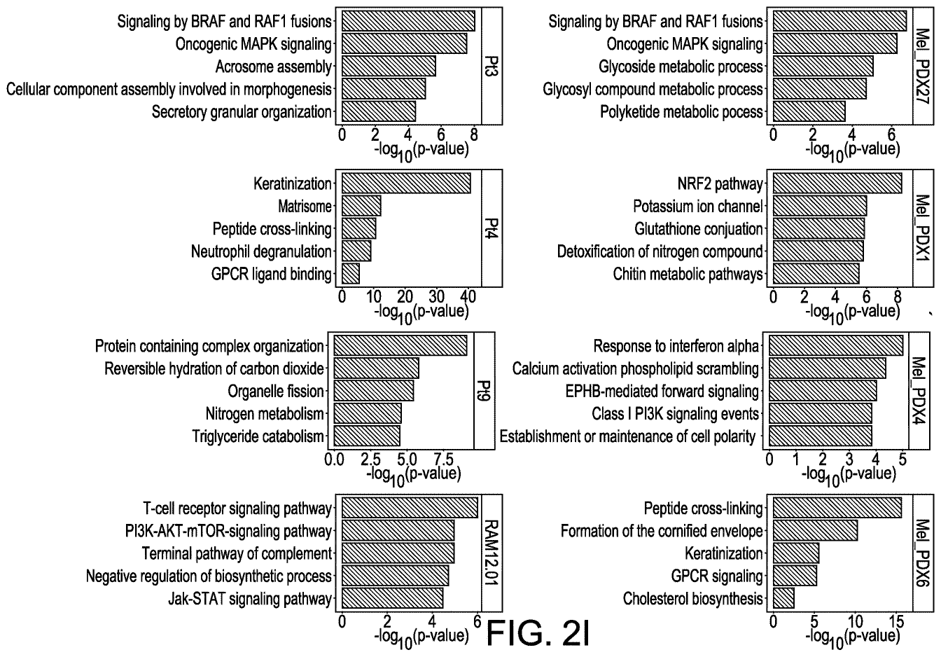

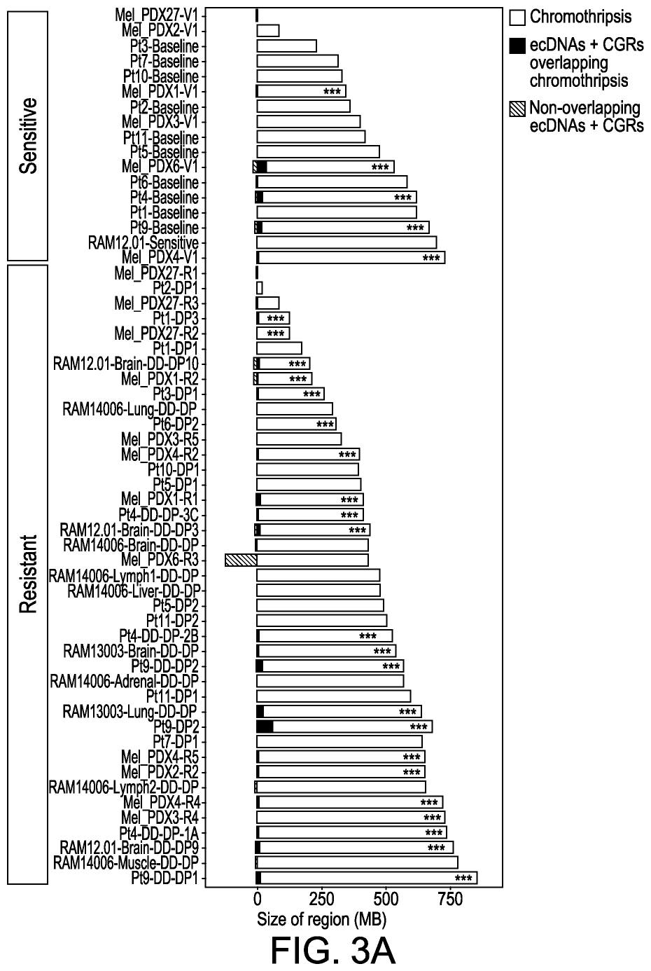

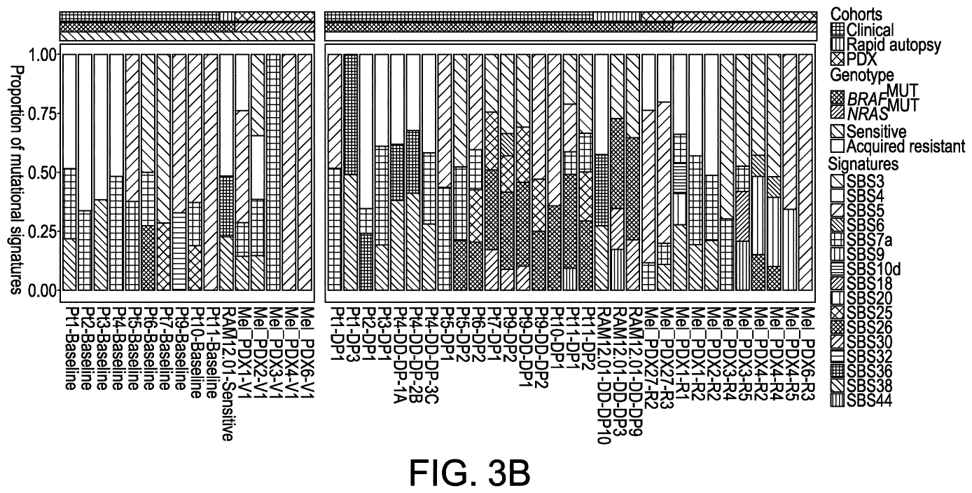

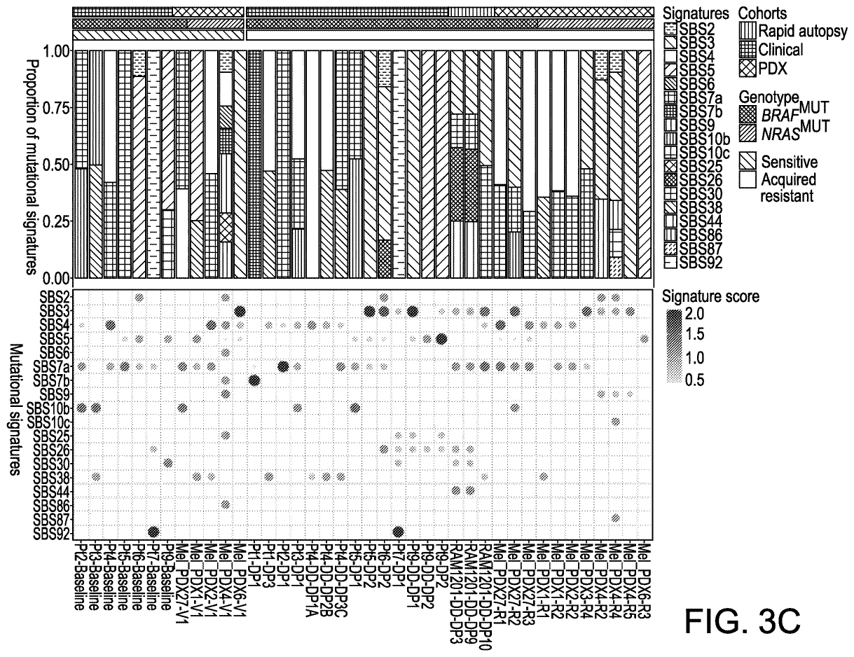

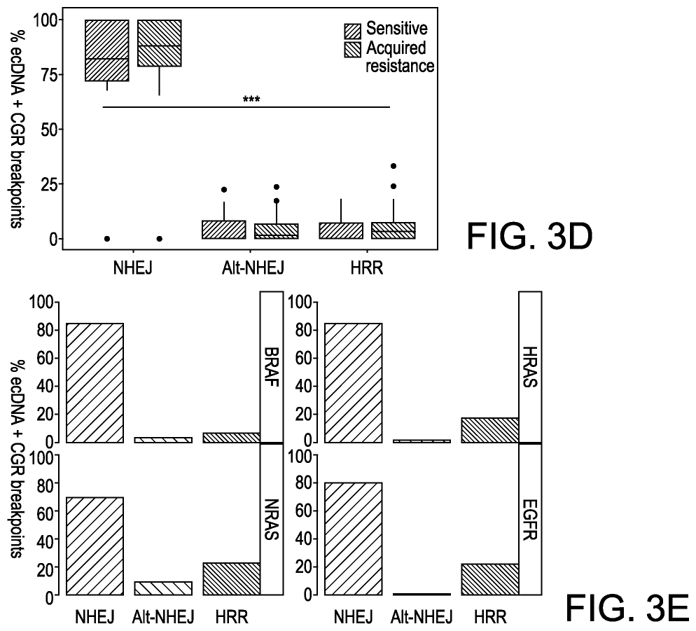

If chromothripsis were a precursor step for ecDNA and CGR generation during melanoma evolution and, in particular, evolution on MAPKi therapy, we expected a non-random overlap of affected genomic spans. Consistent with expectation, we observed that the genomic spans of ecDNAs and CGRs overlapped significantly or non-randomly with chromothriptic regions in ~29% (5 of 17) of MAPKi-sensitive/naive genomes and in ~54% (22 of 41) of acquired MAPKi-resistant genomes (FIG. 3A). For every genome, we computed the sizes of ecDNAs+CGRs (a−x), chromothriptic regions (b−x), their overlap (x), and genomic regions devoid of these genomic alterations (g−a−b). These four quantities were represented in 2×2 contingency tables to carry out the Fisher's exact test. Using hypergeometric distribution, we then estimated the probability of observing random overlaps between ecDNA+CGR spans and chromothriptic spans. In each tumor genome with overlaps between ecDNA+CGR and chromothriptic regions, we observed a significant non-random convergence (Fisher's exact test, p<0.00001), suggesting chromothripsis as a potential origin of ecDNAs and CGRs (FIG. 3A).

Tumor Mutational Burdens and Single-Base Substitution (SBS) Signatures of Chromothripsis, ecDNAs, and CGRs

Chromothripsis generation is hypothesized to involve micronuclei, which expose entrapped chromosomes to mutagens and predispose them to replication, transcription as well as DNA repair defects (31). Therefore, we identified and characterized the single-nucleotide variants (SNVs) within the chromothriptic regions across the melanoma genomes in our three cohorts. The numbers of chromothripsis-associated SNVs ranged from 31 to 2526 per Mb (mean 154 mutations/Mb, median 96 mutations/Mb). We next tested the hypothesis that chromothripsis may disproportionally contribute to the tumor mutational burdens (TMBs), especially in acquired-resistant genomes, by calculating the numbers of SNVs within chromothriptic regions relative to the numbers of SNVs within non-chromothriptic regions. Acquired-resistant tumors harbored an average of 195 SNVs/Mb in chromothriptic regions versus an average of 93 SNVs/Mb in non-chromothriptic regions. Since the ratio of chromothriptic region (~476 Mb) to non-chromothriptic region (~2619 Mb) is 0.18 and given the TMB of 170 SNVs/Mb in acquired-resistance, we expected only 31 SNVs/Mb (0.18×170) in chromothriptic regions of acquired-resistant tumors, if SNVs were evenly distributed across the genome. Moreover, MAPKi-sensitive or pretreatment tumors harbored an average of 121 SNVs/Mb in chromothriptic regions versus an average of 84 SNVs/Mb in non-chromothriptic regions. Again, an observed average of 121 SNVs/Mb in chromothriptic regions is higher than the expected 20 SNVs/Mb in the chromothripsis regions, which was calculated based on a 0.17 ratio of chromothriptic region (~464 Mb) to non-chromothriptic region (~2631 Mb) and the observed average TMB of 115 SNVs/Mb in MAPKi-sensitive tumors. Hence, in both acquired-resistant and MAPKi-sensitive melanoma genomes, chromothriptic regions are enriched for SNVs compared to non-chromothriptic regions (Fisher's exact test, p<0.00001). Furthermore, the ratio of SNVs in chromothriptic to non-chromothriptic genomes is higher in acquired-resistant versus MAPKi-naive/sensitive tumors (Wilcoxon rank sum test, p=0.005) (FIG. 9C), suggesting that MAPKi-selected chromothriptic regions in some individuals feature a mutator phenotype that is further accelerated.

We have reported that MAPKi selection of clinical melanoma alters the mutational spectra (9). To characterize how a MAPKi-associated mutator phenotype affects chromothriptic genomic spans selected by MAPKi, we culled chromothripsis-associated somatic mutations unique to MAPKi-sensitive/naive (64 SNVs/Mb) versus -resistant genomes (58 SNVs/Mb) and analyzed signatures of SBS (FIG. 3B), double-base substitutions (DBSs), and indels (IDs) (FIG. 9D) (32,33). In both MAPKi-sensitive/naive and acquired-resistant tumors, we frequently detected signatures of tobacco smoking (SBS4) and ultraviolet radiation (SBS7a, SBS38). In both MAPKi-sensitive/naive and acquired-resistant tumors, we also detected a signature of defective HRR (SBS3), and this trended higher in resistant (12 of 31) versus sensitive (3 of 16) tumors, although this difference was not significant. Unique but infrequent to acquired MAPKi-resistance was the detection of mutational signatures of polymerase eta somatic hypermutation (SBS9) and defective POLD1 proofreading activity (SBS10d, SBS20). Unique and recurrent to acquired MAPKi-resistance was the detection of mutational signatures of defective DNA mismatch repair (MMR) (SBS6, SBS20, SBS26, SBS44; 14 of 31 resistant tumors; 10 of 16 patients) and of defective base excision repair (BER) [or of damage due to reactive oxygen species (ROS) or mutations in NTHL1 and MUTYH](SBS18, SBS30, SBS36), except that SBS26 and SBS36 signatures were also detected at a low percentage in two sensitive tumors (one of which has been exposed to MAPKi). BER/ROS signatures were observed more frequently in the BRAF^{V600MUT} (10 of 22 resistant tumors, 6 of 11 patients) than the NRAS' (1 of 9 resistant tumors, 1 of 5 patients) subset. This differential frequency maybe due to the longer MAPKi exposure in the clinical (versus PDX or experimental) setting. Consistently, BER mutational signature (SBS18) was detected in Mel_PDX3-R5, which was among the acquired-resistant PDX tumors with longer durations of MAPKi treatment (FIG. 8A). Overall, resistance-specific (versus sensitivity-specific) chromothriptic SBSs enriched for signatures of defects in BER (Wilcoxon rank sum test, p=0.04) and in MMR (Wilcoxon rank sum test, p=0.005). Moreover, resistance-specific (versus sensitivity-specific) non-chromothriptic SBSs also enriched for signatures of defects in BER (Wilcoxon rank sum test, p=0.05). MMR (Wilcoxon rank sum test, p=0.008) as well as HRR (Wilcoxon rank sum test, p=0.04). We did not identify any resistance-specific or -enriched DBS and ID signature (FIG. 9D).

We also identified SBS signatures within ecDNA and CGR sequences and determined whether certain SBS signatures are enriched in acquired MAPKi-resistant (versus MAPKi-sensitive/naive) tumors or ecDNA-/CGR-amplicons (versus non-involved genomic regions, regardless of tumor sensitivity or resistance) (FIG. 3C). We extracted SNVs unique to MAPKi-sensitive/naive (n=12) and acquired-resistant (n=28) tumors (in a patient-matched fashion) within ecDNA- and/or CGR-amplicons. CGR+ecDNA-associated TMB trended higher in acquired-resistant (130 SNVs/Mb) compared to MAPKi-sensitive/naive (80 SNVs/Mb) tumors (Wilcoxon rank sum test, p=0.06). Enrichment of SBS3 (defective HRR) was more recurrent in resistant tumors (11 of 28) compared to patient-matched sensitive tumors (1 of 12), although this difference did not reach statistical significance (Wilcoxon rank sum test, p=0.08) (FIG. 3C). Moreover, we derived mutational signature enrichment scores by calculating the normalized ratios of signature proportions within ecDNAs and/or CGRs and within regions devoid of these events (scores>1 defined as positive enrichment; see Methods). We found that 25% (7 of 28) of acquired-resistant tumors display positive enrichment scores for defective HRR signatures (SBS3) within ecDNAs and CGRs, while only 8% (1 of 12) of sensitive tumors displayed positive enrichment (Wilcoxon rank sum test, p=0.3) (FIG. 3C). In either sensitive or resistant tumors, we observed positive enrichment scores for SBS signatures reflective of APOBEC cytidine deaminase activity (SBS2; 2 of 12 in sensitivity; 3 of 28 in resistance) and polymerase epsilon exonuclease domain mutations (SBS10b; 3 of 12 in sensitivity; 3 of 28 in resistance). Positive enrichment scores were also noted with lower recurrence for SBS signatures of defective POLD1 proofreading (SBS10c), defective MMR (SBS44), chemotherapy treatment (SBS86, SBS87), and tobacco smoking (SBS82). Overall, 50% (6 of 12) of sensitive/naive and 32% (9 of 28) of acquired-resistant tumors display enrichment of unique SBS signatures within ecDNA+CGR sequences compared to uninvolved genomic regions. Lastly, we addressed whether enrichment of unique SBS signatures differed within ecDNAs versus CGRs in acquired resistance (FIG. 9E). We observed positive enrichment scores of SBS2 (APOBEC cytidine deaminase activity) in 26% (5 of 19) of ecDNA⁺ acquired-resistant tumors, compared to 4% (1 of 24) of CGR⁺ acquired-resistant tumor (Wilcoxon rank sum test, p=0.05). This finding is consistent with the recent discovery of the co-occurrence of APOBEC3 kataegis in ecDNAs (33). We also observed positive enrichment scores of SBS3 (defective HRR) in 47% (9 of 19) of ecDNA⁺ acquired-resistant tumors, compared to 17% (4 of 24) of CGR⁺ acquired-resistant tumor (Wilcoxon rank sum test, p=0.07) (FIG. 9E). In short, ecDNA and CGR amplicons, whether in MAPKi-sensitive/naive or acquired-resistant tumors, harbor SBS patterns suggestive of specific defects or deficiencies in HRR. In acquired MAPKi-resistance, APOBEC activity likely contributes to ecDNA mutagenesis and hence shapes the mutanome of ecDNA-amplicons. Further analysis might lead to insights into ecDNA biogenesis and suppressive strategies.

NHEJ Underlies the Formation of ecDNAs and CGRs

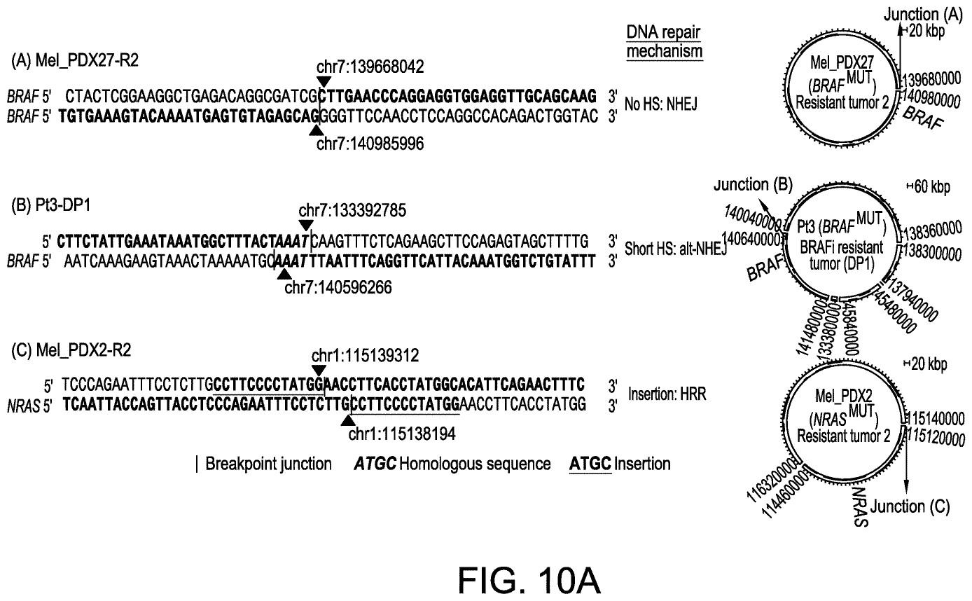

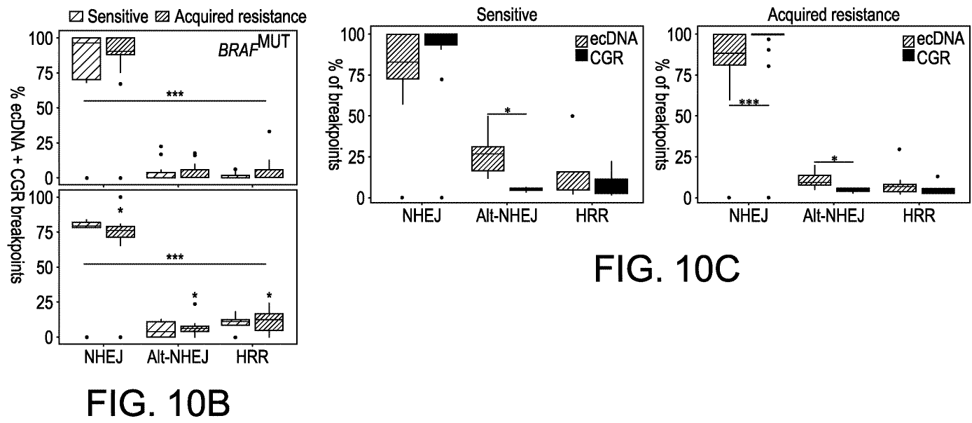

We analyzed the breakpoint-junctional sequences of CGRs and ecDNAs to infer DSB repair processes underlying resistance-associated amplicons (FIG. 3D). Alternative end-joining refers to mechanisms of DSB repair that may compensate for HRR- and NHEJ-based repairs and comprises of single-strand annealing (SSA), microhomology-mediated end-joining (MMEJ), and other end-joining pathways (34). SSA is indicated in the breakpoint junctions by complementary repeat sequences>25 nucleotides, whereas MMEJ by shorter tracks of sequence homology (2-20 nucleotides). Moreover, replicative processes, such as fork stalling template switching and MMEJ, can contribute to the generation of CGRs. Breakpoint junctions derived from replicative processes (e.g., replicative fork collapsing when it encounters a nick) are expected to have microhomologies, insertions, and relatively long templated insertions (35,36). Indeed, signatures of replication processes and templated insertion, as well as that of NHEJ, were detected pan-cancer (12). Analysis of breakpoint-junctional sequences of all resistance- and sensitivity-associated CGRs and ecDNAs inferred NHEJ as the main mechanism of double-stranded DNA fragment joining or rearrangement (FIG. 3D and FIG. 10A). A lower number of breakpoint sequences displayed short and long homologous sequences as well as insertions (>10 bp), which suggested DSB repair by alt-NHEJ and HRR, respectively (FIG. 3D). Analysis of breakpoint-junctional sequences of specifically ecDNAs and CGRs harboring MAPK-reactivation or MAPKi resistance-driver genes also revealed a similar pattern, with NHEJ dominating the landscape of DSB repair mechanisms (FIG. 3E). Of unknown significance or etiology, a higher incidence of double-stranded DNA ligation by HRR was detected in NRAS^{MUT} melanoma, either sensitive or acquired-resistant to MAPKi, compared to BRAF^{V600MUT} melanoma (FIG. 10B). In acquired-resistant genomes, we also detected a higher incidence of double-stranded DNA ligation by NHEJ for CGRs, compared to ecDNAs (FIG. 10C).

DNA-PKi and/or PARPi Prevent Acquired MAPKi-Resistance in Melanoma Cell Lines

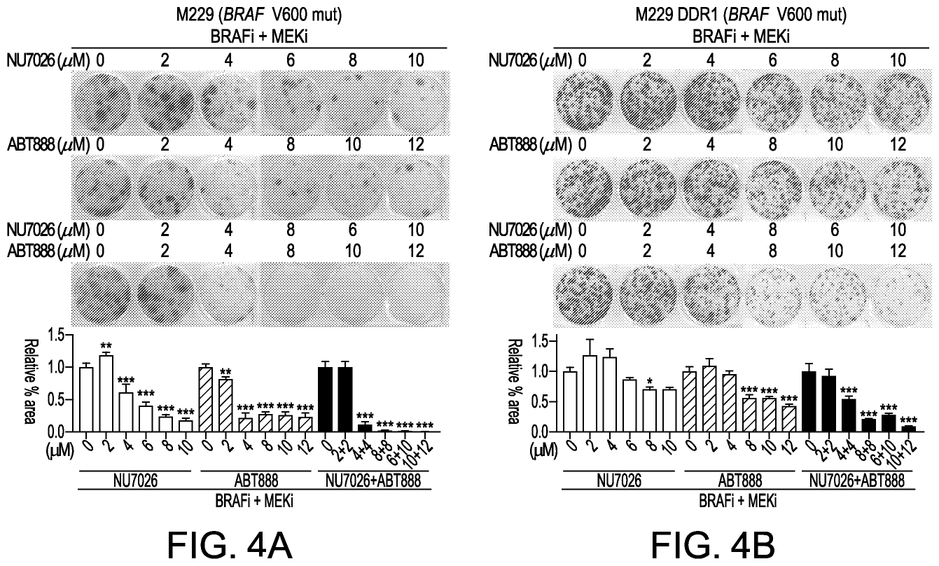

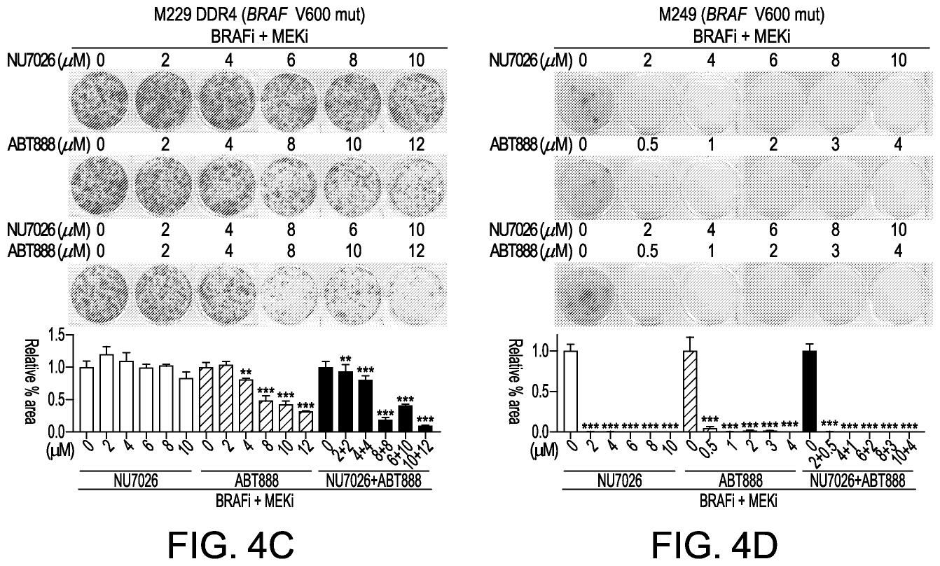

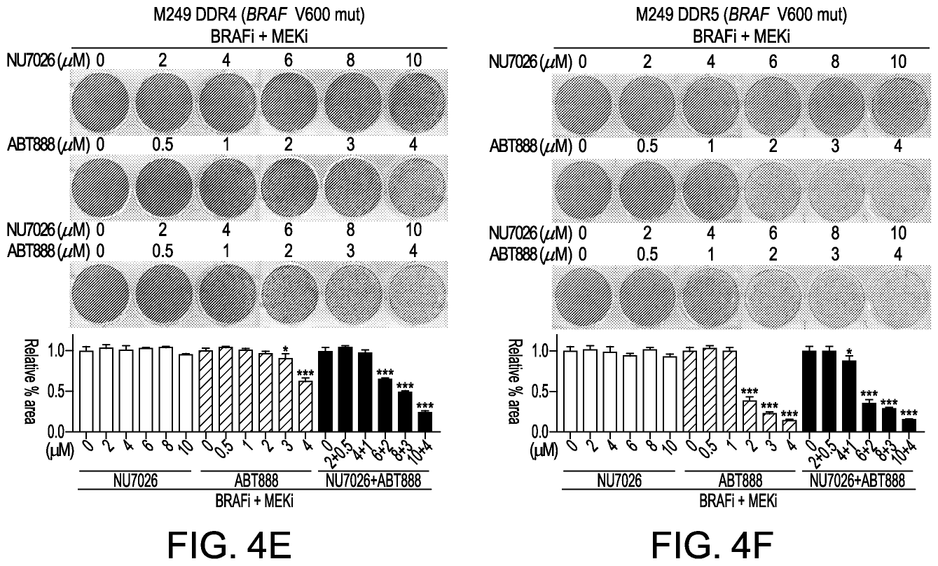

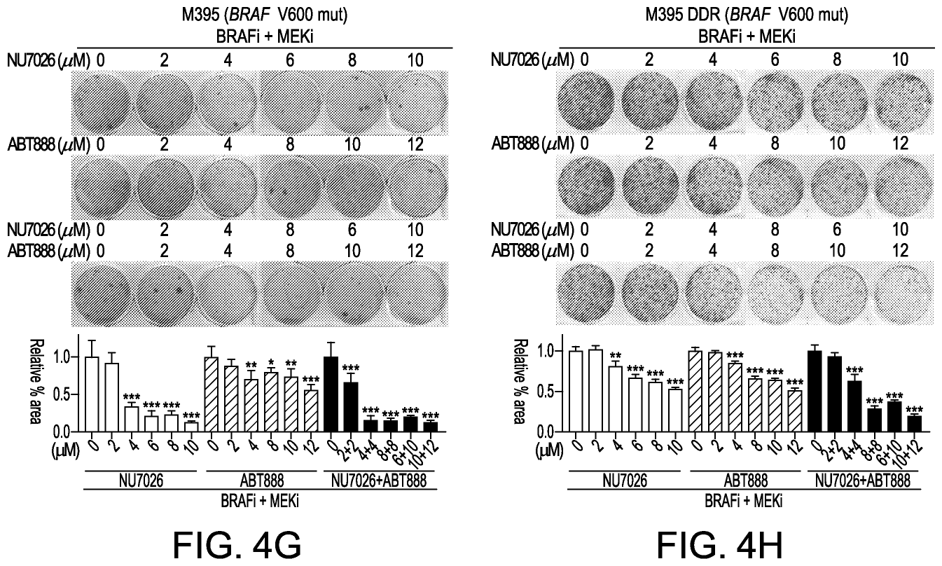

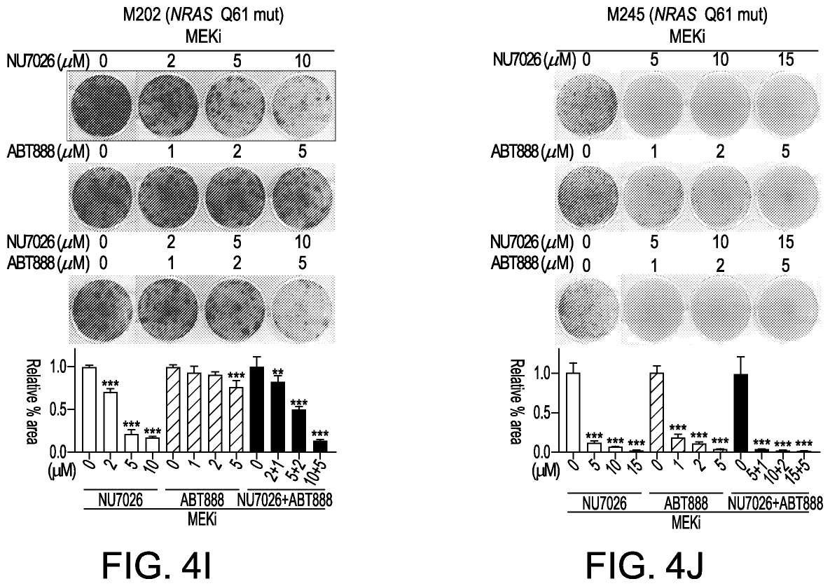

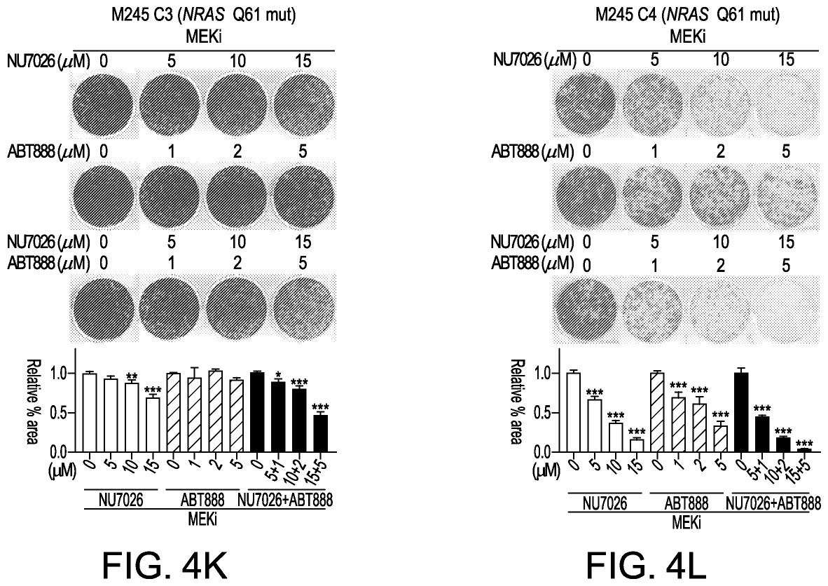

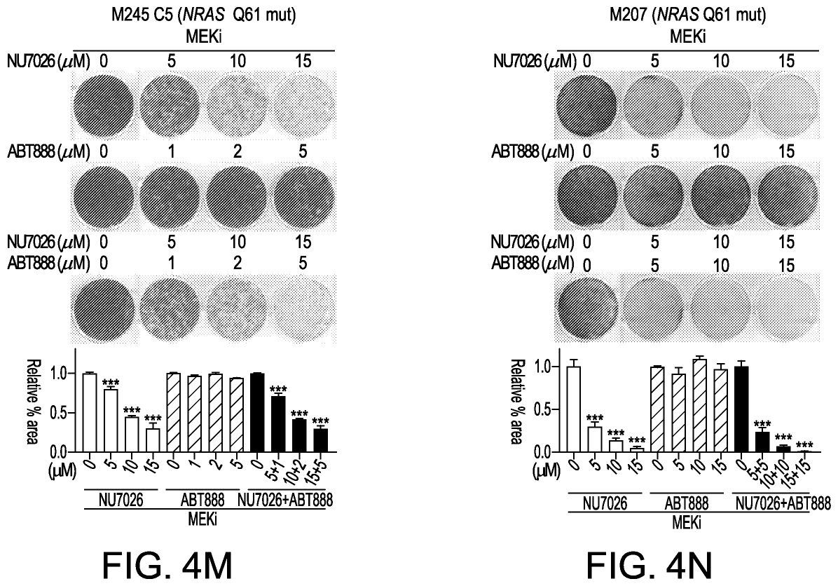

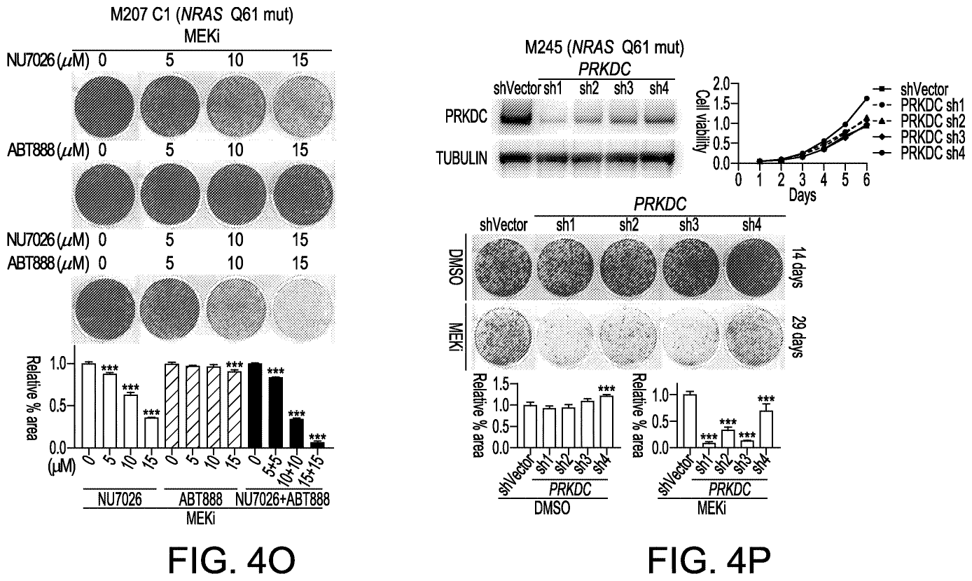

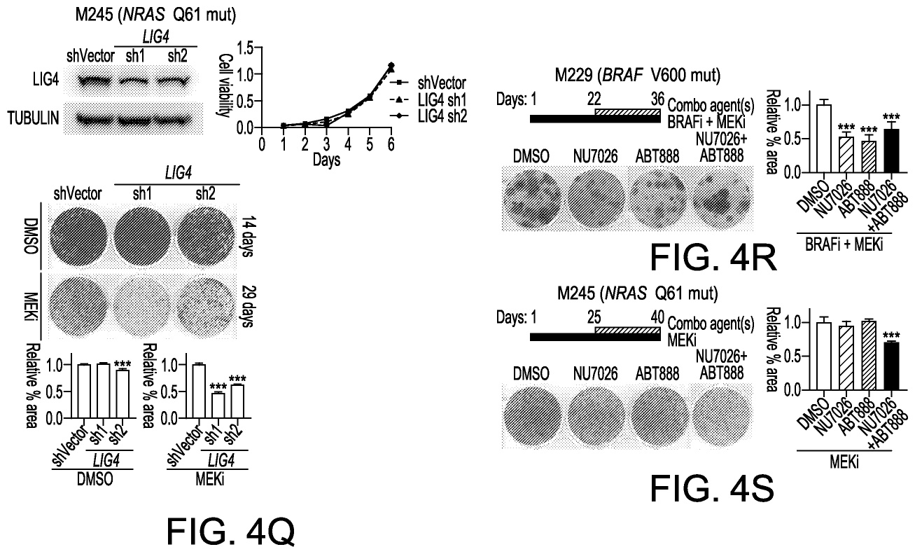

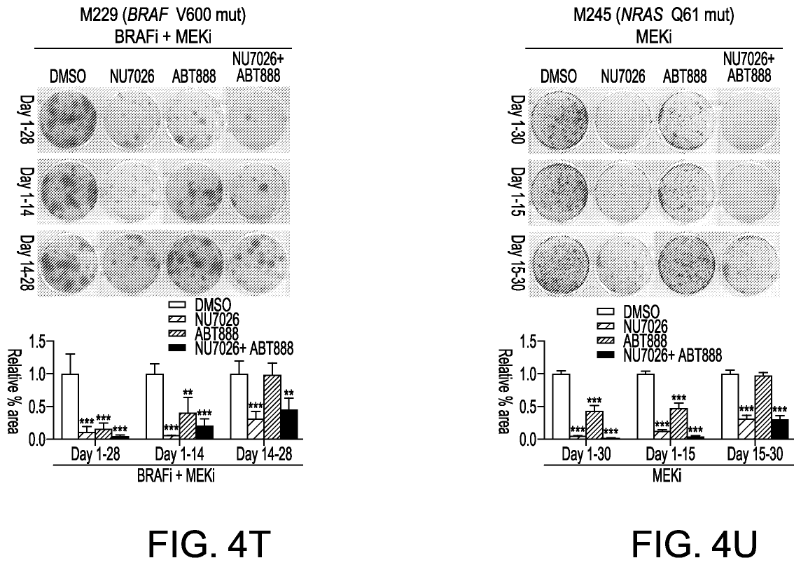

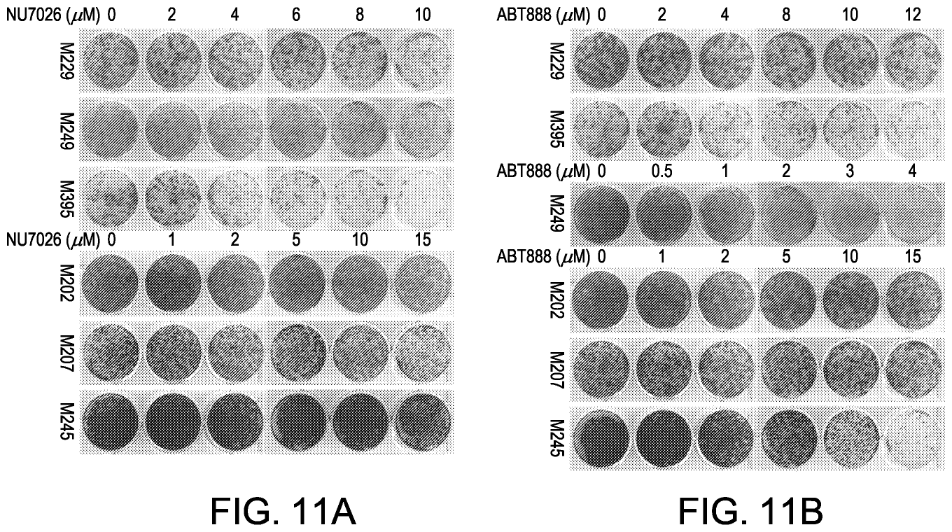

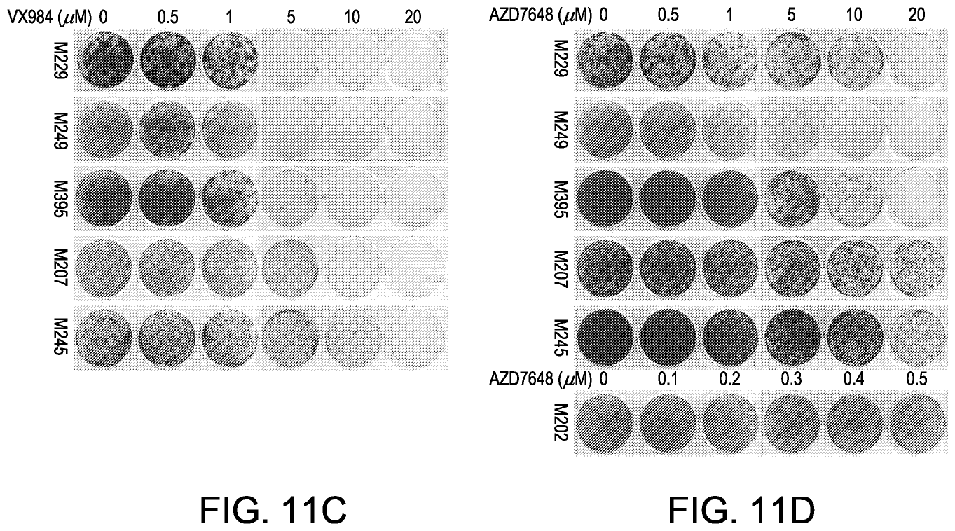

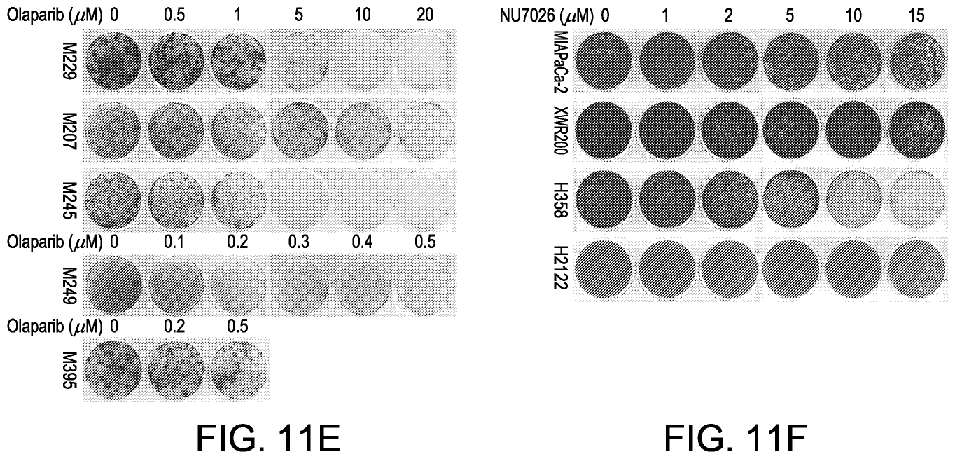

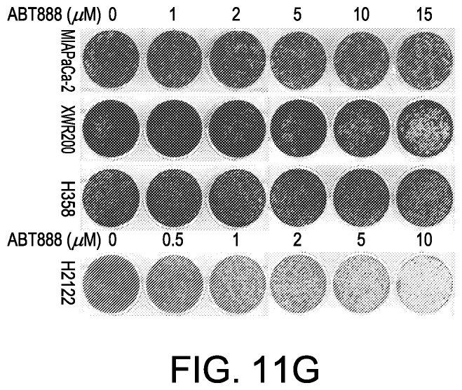

DNA-dependent protein kinase catalytic subunit (DNA-PK_{CS}) and PARP1/2 are involved in multiple DNA DSB repair pathways, particularly NHEJ (DNA-PK_{CS}), HRR (DNA-PKcs, PARP1/2), and MMEJ (PARP1/2) (37,38). Thus, we tested the activity of the specific DNA-PK_{CS} inhibitor (DNA-PKi) NU7026 and PARP1/2 inhibitor (PARPi) ABT888 (veliparib), individually and combinatorially, in preventing the clonal emergence of drug-tolerant proliferating persisters (DTPPs) (39,40) from human BRAF^{V600MUT} (n=3; M229, M249, M395) or NRAS^{Q61MUT} (n=3: M202, M207, M245) (FIG. 4) melanoma cell lines chronically treated with BRAFi+MEKi or MEKi, respectively. Given the hypothesis that DNA-PKi interferes with MAPKi-elicited, de novo rearrangement of specific SVs including ecDNAs and CGRs, we hypothesized that DNA-PKi is more effective at preventing rather than overcoming or reversing resistance, once established by chronic MAPKi treatment. Hence, we also tested treatments with NU7026 and/or ABT888 in combination with MAPKi in acquired MAPKi-resistant sub-lines (n=9) that are isogenic to the human BRAF^{V600MUT} and NRAS^{Q61MUT} parental cell lines (FIG. 4A to 40). BRAF^{V600MUT} sub-lines with acquired resistance to BRAFi+MEKi are annotated as double-drug resistant (DDR), while NRAS^{MUT} sub-lines with acquired resistance to MEKi are annotated simply by their clone (C) numbers. We began by testing the single-agent anti-clonogenic growth activity of NU7026 and ABT888 on parental melanoma cell lines without MAPKi treatment, in order to select non-inhibitory concentrations to test in combination with MAPKi (FIGS. 11A and 11B). In combination with MAPKi, NU7026 synergistically and dose-dependently prevented DTPP formation in all parental cell lines tested (FIGS. 4A, 4D, 4G, 4I, 4J, and 4N), while ABT888 displayed activity in 3 of 6 parental cell lines (FIGS. 4A, 4D, and 4J). In cases where ABT888 individually was active in preventing DTPP formation, NU7026 plus ABT888 led to even greater suppression of acquired MAPKi resistance (FIGS. 4A, 4D, and 4J). Consistently in all acquired MAPKi-resistant sub-lines, NU7026 (or ABT888) at the lower range of the concentrations tested displayed no or reduced anti-clonogenic activity, compared to their activities observed in isogenic parental lines (FIG. 4A to 4O). More recent generations of DNA-PKi's display improved selectivity and potency (37,41). Hence, we used next-generation DNA-PKi's (VX984 and AZD7648) and PARPi (olaparib) and corroborated the combinatorial efficacy of DNA-PKi and PARPI in parental cell lines (FIG. 11C to 11E and FIG. 12). In M245, we further corroborated the pharmacologic findings with genetic studies. Using independent shRNAs against two critical NHEJ genes (PRKDC, which encodes DNA-PK_{CS}, and DNA ligase IV or LIG4), we showed that their protein knockdown, while not having a significant effect on growth without MAPKi, strongly suppressed the frequency of DTPP emergence (FIGS. 4P and 4Q).

The acquired resistant sub-lines used in the prior experiments have been adapted chronically (i.e., months to years) to MAPKi. Hence, we tested whether a shorter delay (i.e., weeks) in the co-treatment of parental cell lines with DNA-PKi (and/or PARPi) would hinder the efficacy in preventing DTPP formation. In both cell lines (M229 BRAF^{V600MUT} and M245 NRAS^{MUT}), we found that a 3- to 3.5-week delay in co-treatment with DNA-PKi and/or PARPi reduced the suppression of resistance (FIGS. 4R and 4S, compared to FIGS. 4A and 4J, respectively). In the same parental cell lines, we then compared co-treatment with DNA-PKi (and/or PARPi) during the first versus second half of the overall MAPKi treatment course (FIGS. 4T and 4U). Notably, we observed that DNA-PKi co-treatment during the first half of the MAPKi treatment course was remititive and during the second half partially remititive. In contrast, the efficacy of PARPi co-treatment was completely lost when administered only during the second half of the MAPKi treatment course. Thus, the upfront and initial phase of MAPKi therapy appears to be the effective window of co-treatment with DNA-PKi (and/or PARPi) to suppress acquired MAPKi resistance, suggesting a preventative mechanism of action.

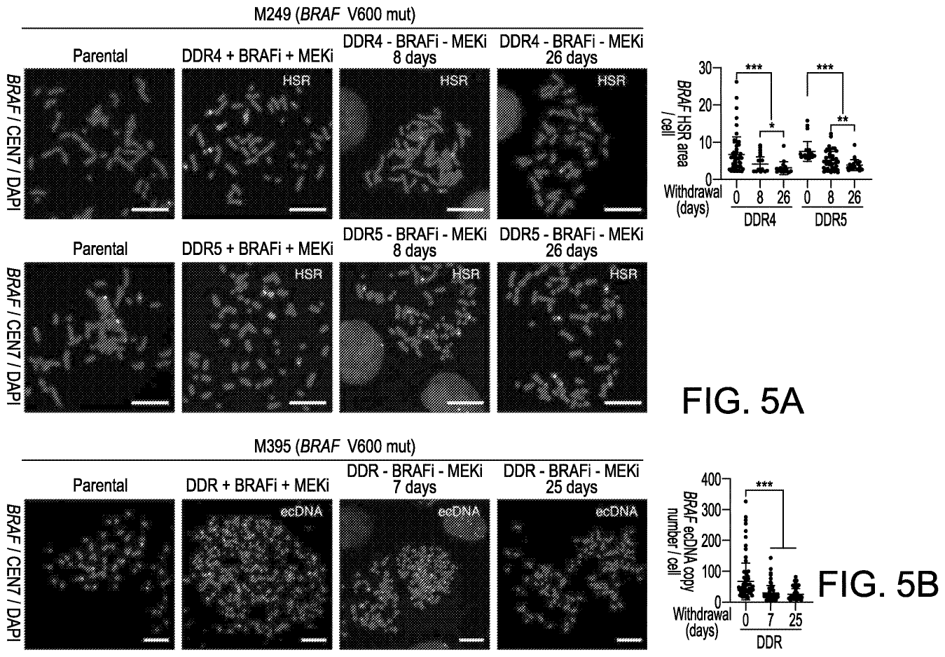

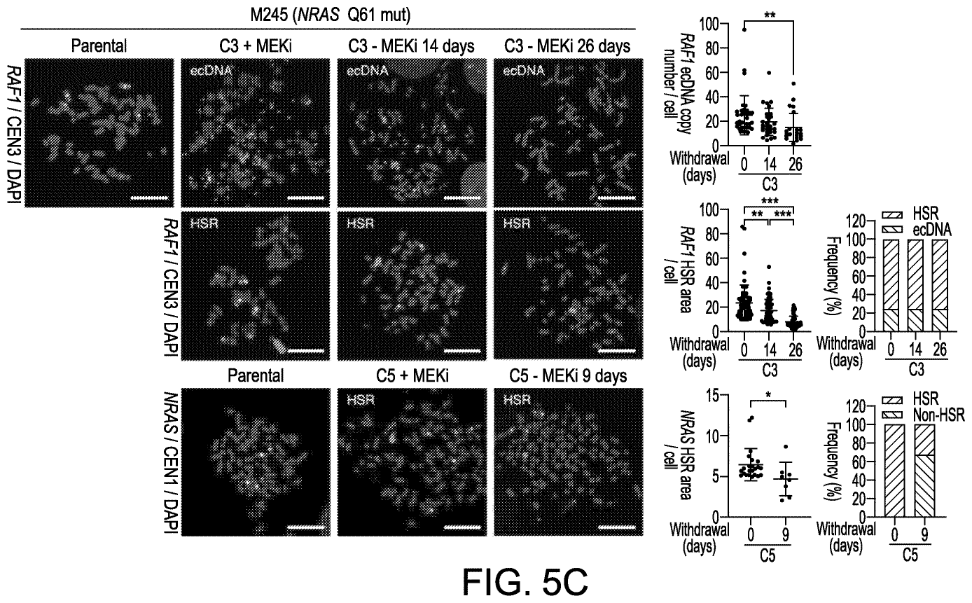

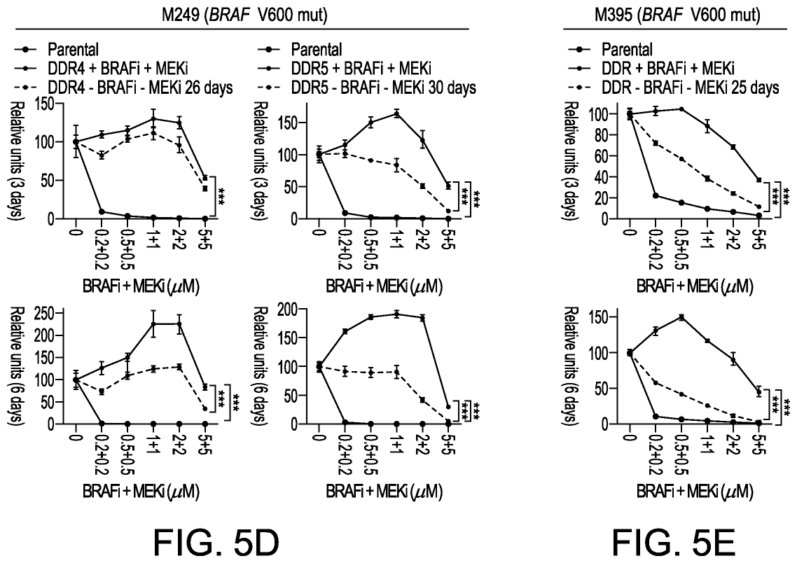

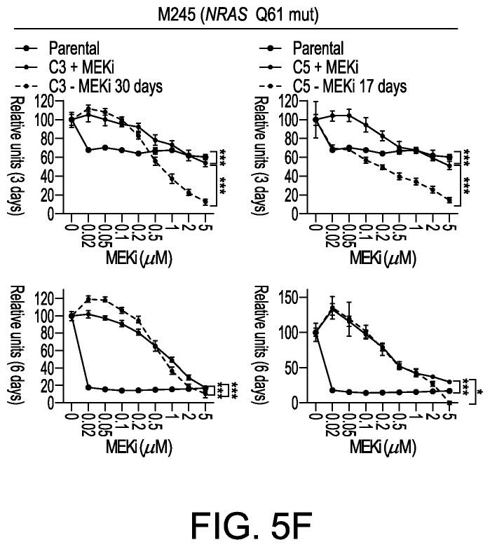

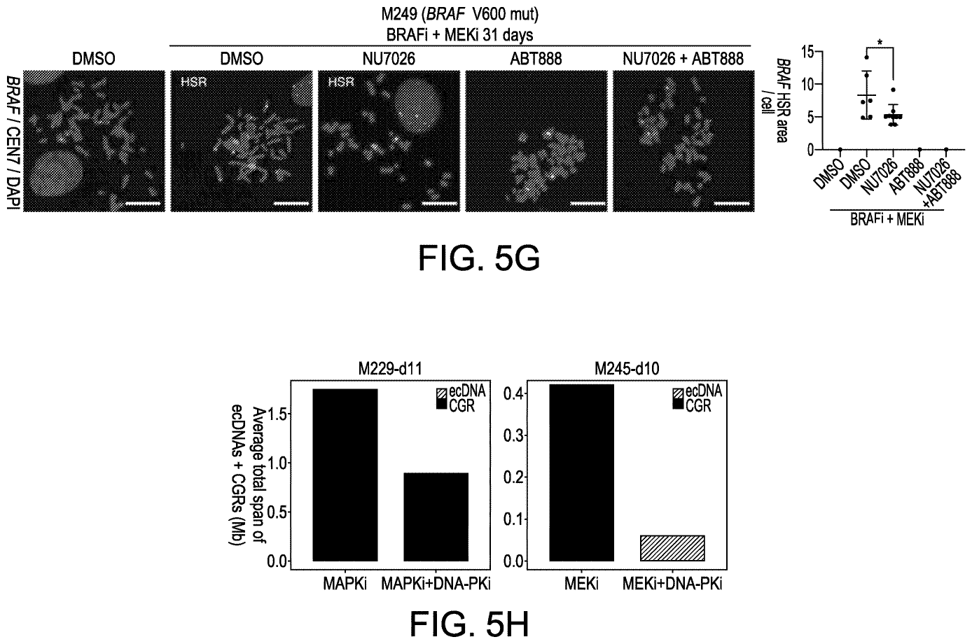

To support further a causal link between acquired MAPKi resistance and ecDNAs or HSRs in human melanoma cell lines, we first sought to determine the modes of amplification of bona fide genes that drive acquired MAPKi resistance. In three parental cell lines (BRAF^{V600MUT}, M249 and M395; NRAS^{MUT}, M245) and five isogenic acquired MAPKi-resistant sub-lines, we performed DNA-FISH on cells in metaphase and probed against BRAF (whose amplification drives acquired MAPKi resistance in BRAF^{V600MUT} melanoma) (5,7-9), RAF1, and NRAS (whose amplification drive acquired MAPKi resistance in NRAS^{MUT} melanoma) (11,22), as well as centromeres of chromosomes 7, 3, and 1, respectively (FIG. 5A to 5C). We observed that BRAF is amplified either as homogeneously staining regions (HSRs) (M249 DDR4 and DDRS) or ecDNAs (M395 DDR); RAF1 as a mixture of HSRs and ecDNAs (M245 C3); and NRAS as HSRs (M245 C5). Expectedly, these driver ecDNAs and HSRs were not detected in the isogenic parental cell lines (FIG. 5A to 5C). We then tested whether MAPKi withdrawal would select against resistant clones with ecDNAs and/or HSRs that amplify the aforementioned resistance-driver genes and, consequently, restore MAPKi sensitivity, at least partially. Importantly, we observed that MAPKi withdrawal from acquired-resistant sub-lines significantly reduced metaphase cells with driver ecDNAs and HSRs (FIG. 5A to 5C) and significantly enhanced their MAPKi sensitivity (FIG. 5D to 5F). Using the M249 parental cell line, which is highly sensitive to resistance suppression by either DNA-PKi or PARPi (FIG. 4D), we tracked the emergence of BRAF HSR early on treatment (day 31) with BRAFi+MEKi or combinations with DNA-PKi, PARPi, or DNA-PKi+PARPi (FIG. 5G). Consistently with BRAF HSRs as a driver of acquired MAPKi-resistance, we observed their induction by BRAFi+MEKi but suppression by combinatorial treatments with DNA-PKi and/or PARPi. During the earliest window on MAPKi treatment, expansion of ecDNA- and CGR-involved genomic spans may be critical for specific resistance-driving ecDNAs and CGRs to emerge later and to provide selective fitness on therapy. We reasoned that DNA-PKi, which is generally more effective than PARPi in preventing acquired resistance (FIG. 4), should suppress MAPKi-elicited, treatment-specific ecDNA plus CGR genomic spans. To test this hypothesis, we performed WGS on two parental cell lines (M229, M245) treated with vehicle, MAPKi, or MAPKi+DNA-PKi for a short duration (10 to 11 days), identified ecDNAs and CGRs, and calculated treatment-specific genomic spans of ecDNAs+CGRs (by filtering out ecDNA and CGR spans detected in vehicle-treated genomes). Consistent with our hypothesis, DNA-PKi co-treatment with MAPKi blunted the expansion of ecDNA+CGR genomic spans in both cell lines (FIG. 5H).

### DNA-PKi Suppresses Acquired MAPKi-Resistance in KRAS G12C PDAC and NSCLC Cell Lines

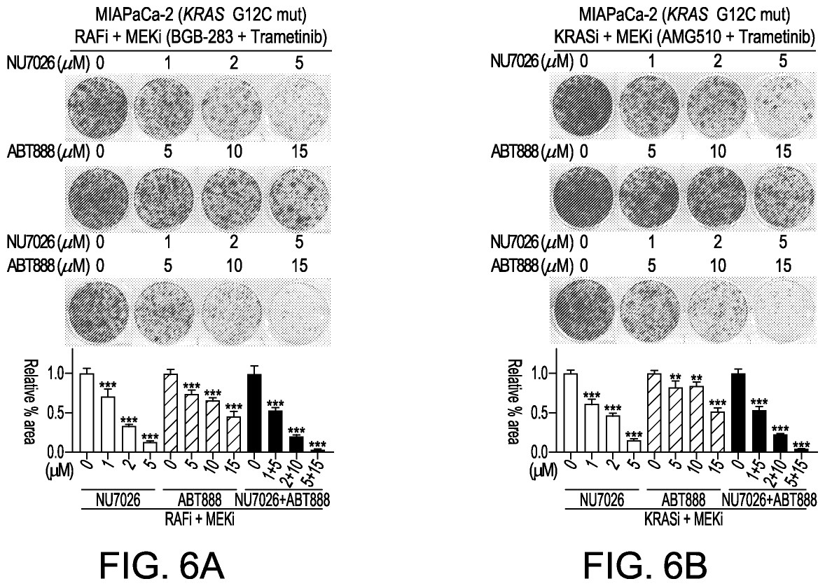

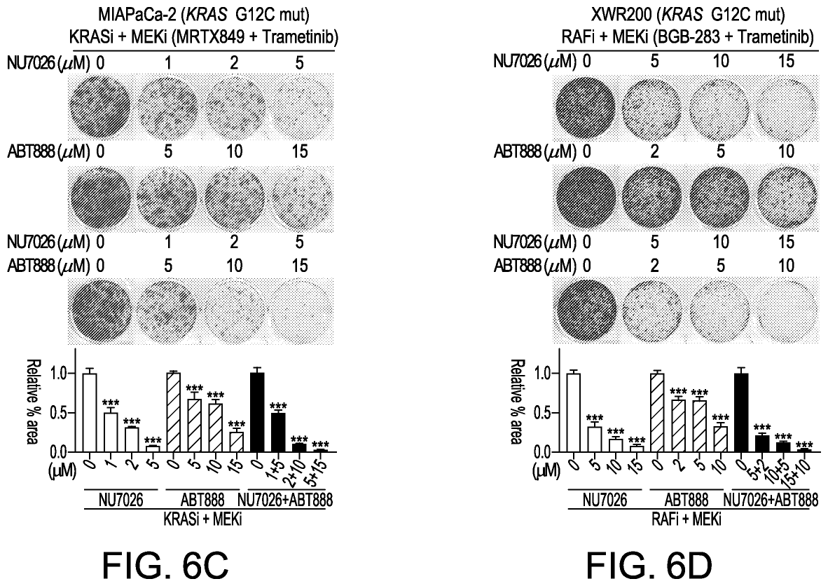

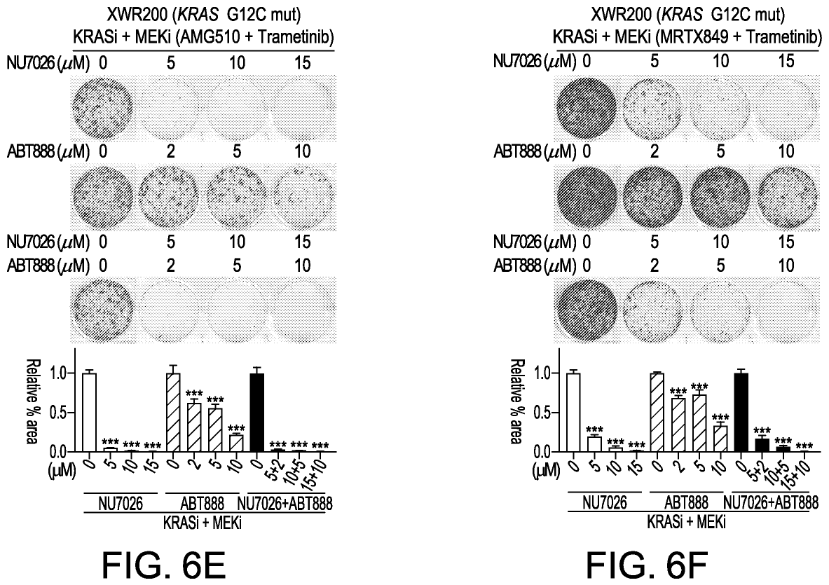

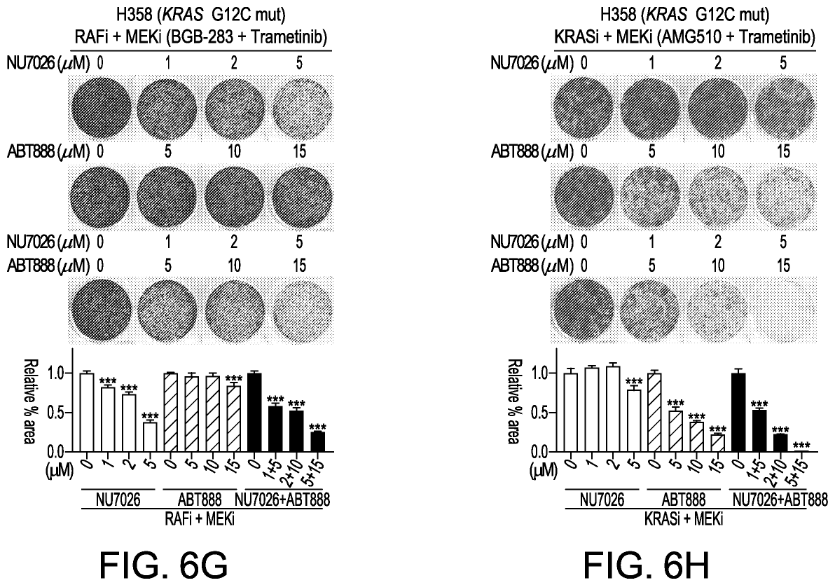

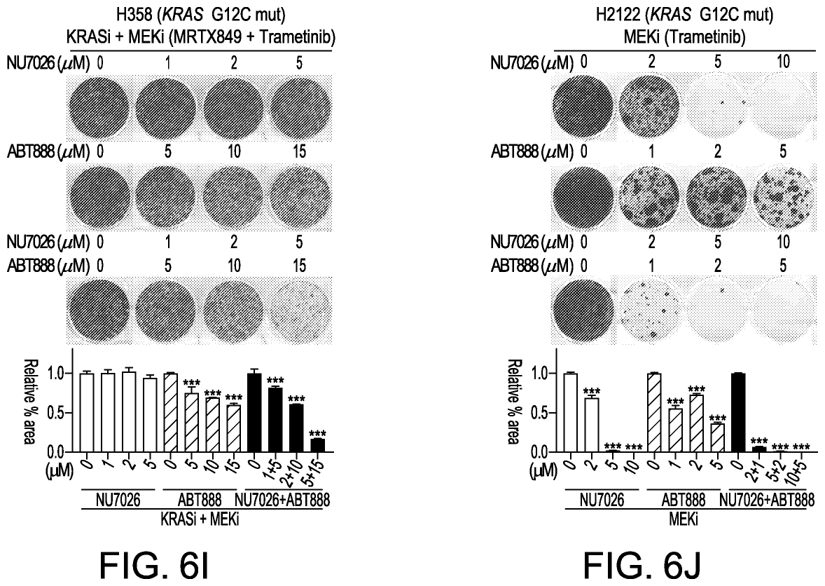

Since DNA-PKi plus MAPKi may constitute an effective combination for BRAF^{MUT} and NRAS^{MUT} melanoma, we explored this combinatorial efficacy (±PARPi) in human pancreatic ductal adenocarcinoma (PDAC) and non-small cell lung carcinoma (NSCLC) cell lines driven by KRAS^{G12C}. For the MAPKi regimen, we used MEKi (trametinib), KRAS G12Ci (AMG510 or MRTX849)+MEKi, or type II RAFi (BGB-283)+MEKi. As with melanoma cell lines, we first identified concentrations of DNA-PKi (NU7026) or PARPi (ABT888) that did not affect clonogenic growth of KRAS^{G12C} PDAC and NSCLC cell lines without MAPKi (FIGS. 11F and 11G). We then assayed for acquired MAPKi resistant growth in these cell lines, without or with DNA-PKi and/or PARPi (FIG. 6). Interestingly, we observed that DNA-PKi effectively suppressed acquired MAPKi resistance in two of two KRAS^{G12C} PDAC cell lines (n=2) (FIG. 6A to 6F) and one of two KRAS^{G12C} NSCLC cell lines (n=2) (FIG. 6G to 6J). Moreover, PARPi was generally ineffective as a combinatorial agent with MAPKi. Future studies should explore the contributions of chromothripsis, ecDNAs, and CGRs to acquired MAPKi resistance in human KRAS^{G12C} PDAC and NSCLC.

DNA-PKi Forestalls Resistance In Vivo and Reduces ecDNA and CGR Size

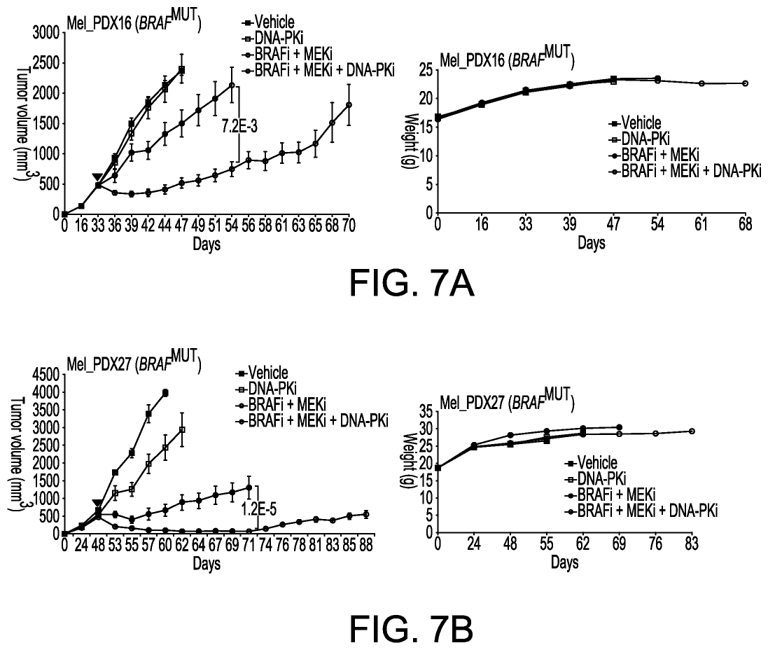

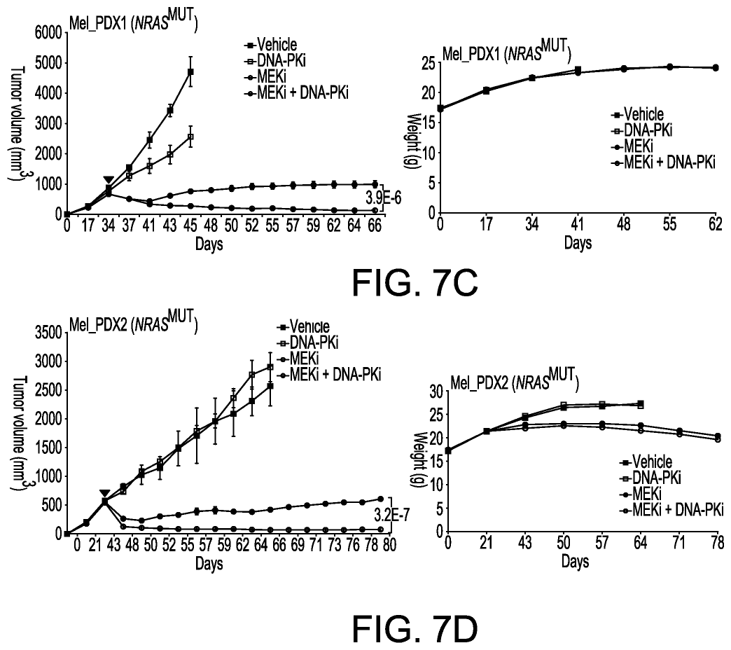

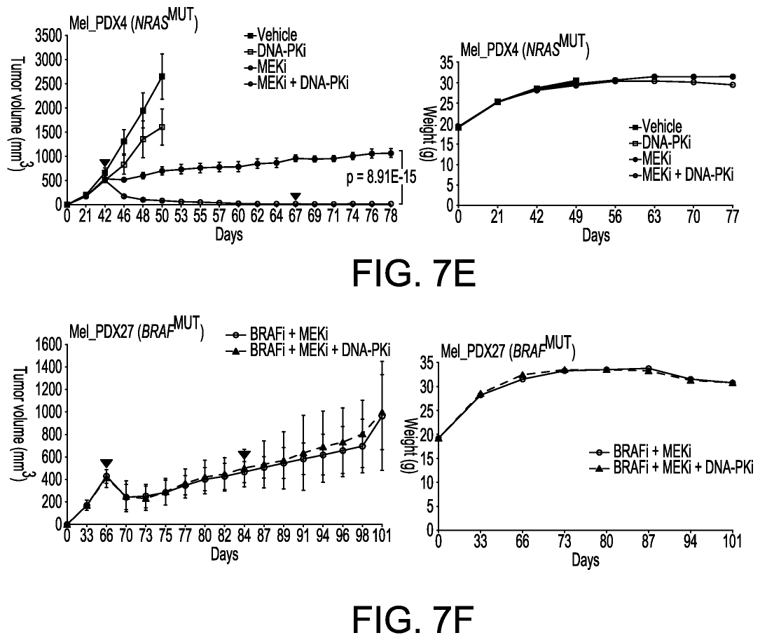

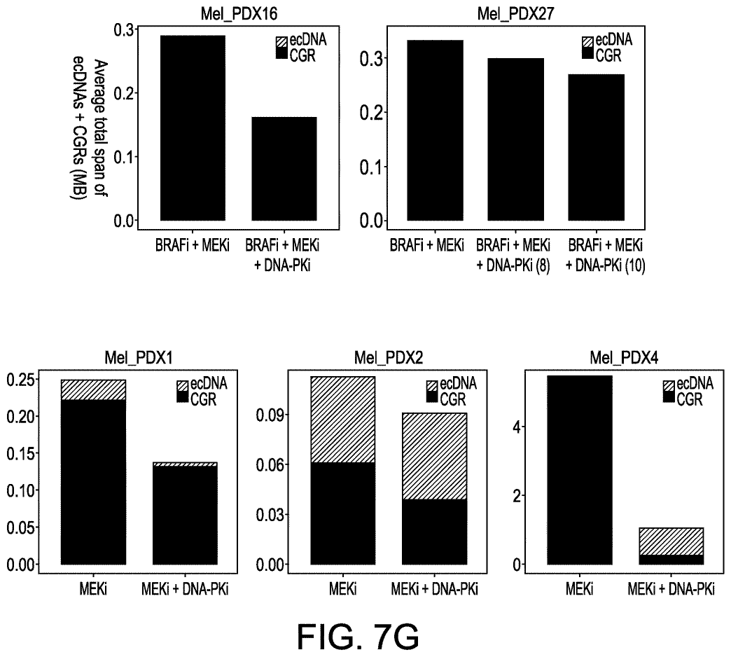

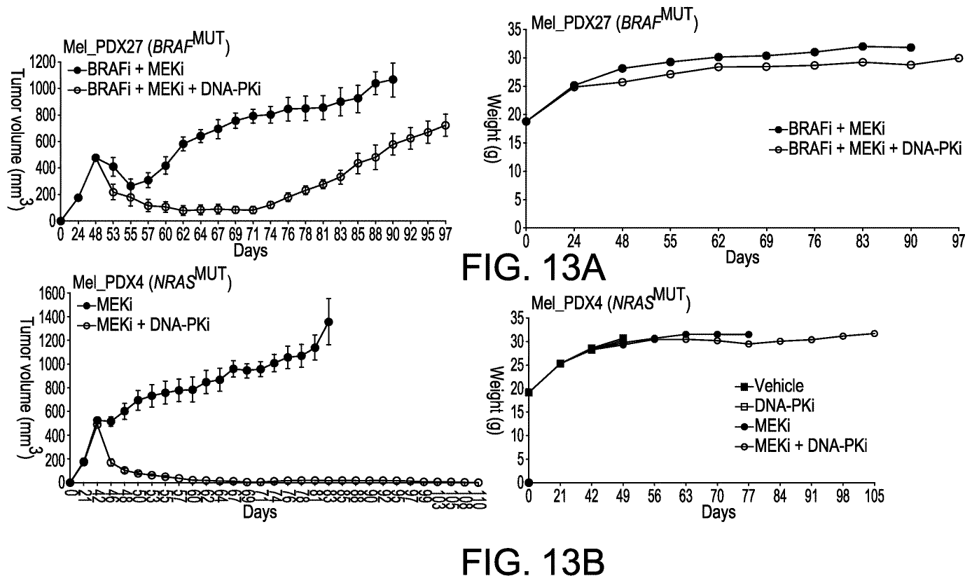

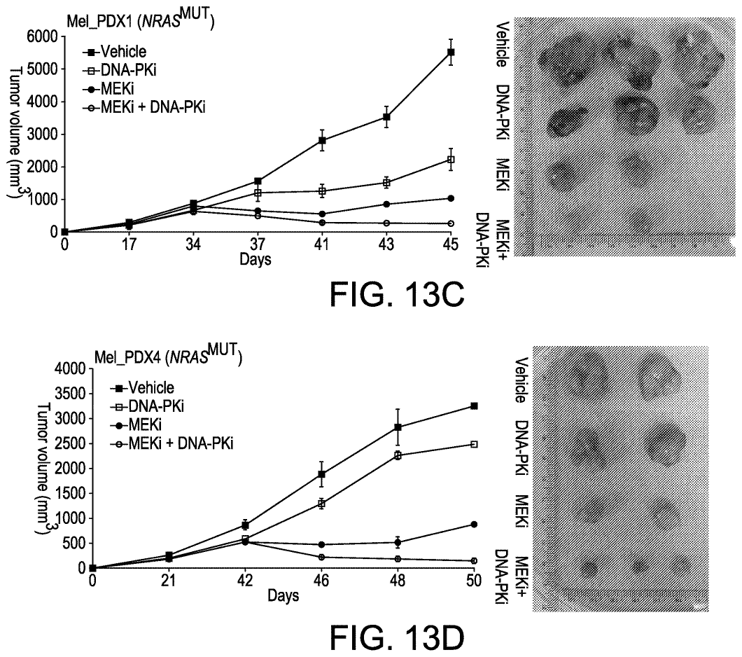

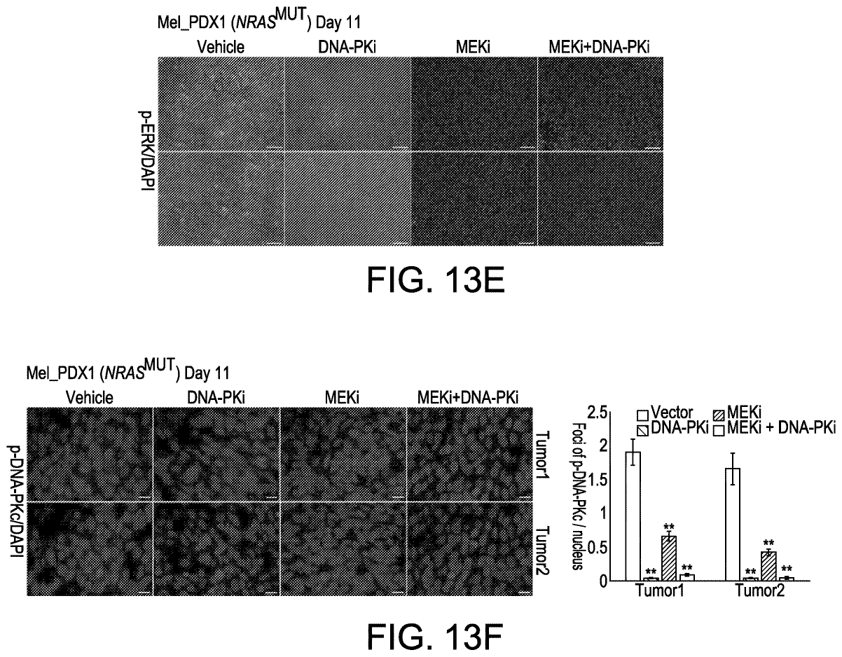

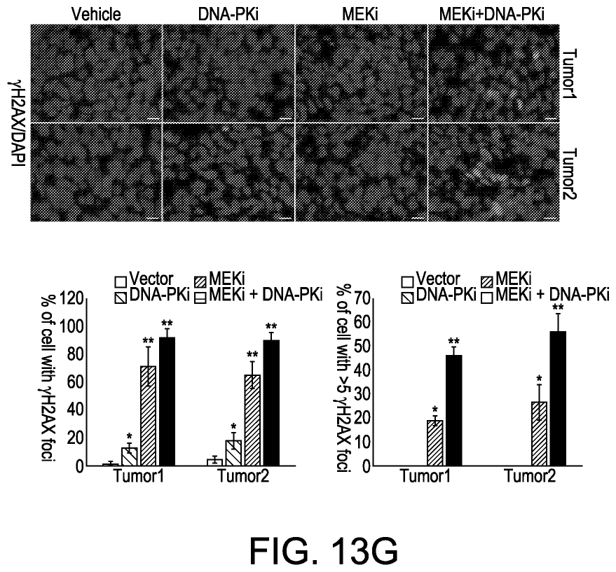

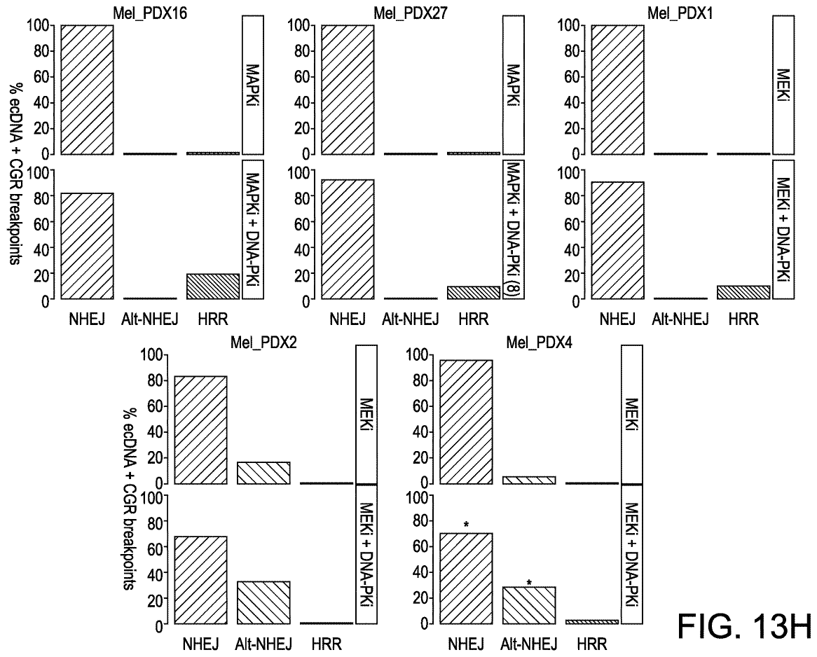

Given the broader resistance-preventative activity of DNA-PKi, we tested NU7026's in vivo efficacy in preventing acquired MAPKi-resistance in five cutaneous melanoma PDXs. In Mel_PDX16 (BRAF^{V600MUT}) melanoma, using well-established tumors (~500 to 700 mm³) (FIG. 7A), BRAFi+MEKi (vemurafenib at 90 mg/kg/d, trametinib at 0.7 mg/kg/d) elicited only tumor growth inhibition, and DNA-PKi treatment (8 mg/kg/d) had no discernable effect on tumor growth compared to vehicle-treated tumors. In contrast, the triplet of BRAFi+MEKi+DNA-PKi elicited tumor regression transiently for ~14 days until the tumors acquired resistance. There were no overt signs of toxicities or a significant reduction in body weight in any experimental group. In Mel_PDX27 (BRAF^{V600MUT}) melanoma (FIG. 7B and FIG. 13A), BRAFi+MEKi (vemurafenib at 90 mg/kg/d, trametinib at 0.7 mg/kg/d) elicited transient tumor regression, while DNA-PKi treatment (8 mg/kg/d) did not elicit tumor regression compared to vehicle-treated tumors. In contrast, the triplet of BRAFi+MEKi+DNA-PKi significantly forestalled acquired resistance, without incurring overt signs of toxicities or a significant reduction in body weight. In NRAS^{MUT} melanoma (Mel_PDX1) (FIG. 7C), DNA-PKi treatment (10 mg/kg/d) did not elicit tumor regression, and MEKi (trametinib, 3 mg/kg/d) elicited only transient tumor regression. In contrast, the combination of MEKi+DNA-PKi led to minimally palpable tumors, without incurring toxicity. In two additional NRAS^{MUT} melanoma PDXs (Mel_PDX2 and Mel_PDX4), we consistently observed superior efficacy of MEKi+DNA-PKi over MEKi alone (FIGS. 7D and 7E). In Mel_PDX4, we initiated dosing with both DNA-PKi at 8 mg/kg/d and MEKi at 3 mg/kg/d but stopped dosing DNA-PKi (in combination with MEKi) on day 67 (FIG. 7E). With longer follow-up (FIG. 13B), discontinuation of DNA-PKi dosing in this group of mice treated initially with MEKi+DNA-PKi did not lead to tumor relapse. Based on cell line findings (FIG. 4), we expected that delayed DNA-PKi co-treatment in vivo would also diminish its preventative mechanism of action. In Mel_PDX27 (BRAF^{V600MUT}) melanoma (FIG. 7B and FIG. 13A), we repeated BRAFi+MEKi (vemurafenib at 90 mg/kg/d, trametinib at 0.7 mg/kg/d) treatment, and, at the time of disease progression (when the mean tumor volume returned to the mean pre-MAPKi volume of ~500 mm³), we divided the tumors/mice into two groups. The first group continued on BRAFi+MEKi treatment at the same dosages, while the second group received DNA-PKi treatment (8 mg/kg/d) added on top of BRAFi+MEKi. Consistent with the cell line results (FIG. 4A to 4O), DNA-PKi co-treatment did not elicit discernible tumor regression on tumors that have already acquired MAPKi resistance in vivo (FIG. 7F).

We then explored the potential early on-treatment pharmacodynamic marker(s) that associate with the superiority of MEKi+DNA-PKi over MEKi alone in forestalling or preventing acquired resistance. We posited that the combination of DNA-PKi with MAPKi (versus MAPKi alone) would suppress the total size of rearranged and amplified gDNA of ecDNAs and CGRs specifically generated due to MAPKi treatment, agnostic of content genes, regulatory elements and their known or putative roles in driving resistance. Using all five PDX models, we collected early on-treatment tumors and vehicle-treated tumors (FIGS. 13C and 13D). Expectedly, p-ERK was suppressed strongly in both groups of tumors treated with MEKi or MEKi+DNA-PKi (FIG. 13E). Binding of the DNA-PK holoenzyme (DNA-PK_{CS}+Ku70+Ku80) to DNA DSBs elicits autophosphorylation of DNA-PK_{CS} serine 2056. We found that DNA-PKi treatment, with or without MEKi, suppressed nuclear p-DNA-PK_{CS} foci (FIG. 13F), which is consistent with NU7026 being able to suppress the kinase activity and autophosphorylation of the activated DNA-PK holoenzyme. Consistent with a recent report that found MAPKi treatment resulting in gH2AX nuclear foci or DSBs in melanoma cell lines (42), we found that short-term MEKi treatment in Mel_PDX1 induced gH2AX nuclear foci in >50% of tumor cells (FIG. 13G). Expectedly, DNA-PKi co-treatment with MEKi further induced DSBs marked by gH2AX foci. We then generated WGS data to enable analysis of CGR and ecDNA amplicons (Table 3). CGR and ecDNA amplicons identified in vehicle-treated PDX tumors were considered as background and were removed from those identified in MEKi- or MEKi+DNA-PKi-treated tumors. Consistent with our hypothesis, MEKi+DNA-PKi (versus MEKi) treatment was associated with reductions in the average total genomic spans of CGRs and ecDNAs in five of five PDX models analyzed (FIG. 7G). We also analyzed the breakpoint-junctional sequences of MEKi treatment-specific ecDNAs and CGRs to infer the relative contributions of DSB repair pathways (FIG. 13H). Consistent with a functional role of DNA-PKi, its combination with MEKi suppressed the contribution of NHEJ, with potentially compensatory increases in alt-NHEJ or HRR processes.

TABLE 3
Frequencies of enriched SBS signatures reflecting
defective DNA repair mechanisms within chromothripsis
and non-chromothriptic regions in MAPKi-sensitive
(n = 16) and acquired-resistant (n = 31) tumors

| | | Defective | | Defective | | Defective | |
|---|---|---|---|---|---|---|---|
| Event | Type | MMR¹ | | BER² | | HRR³ | |
| Chromothripsis | Sensitive | 6% | (1) | 6% | (1) | 19% | (3) |
| | (n = 16) | | | | | | |
| | Resistance | 48% | (15) | 32% | (10) | 39% | (12) |
| | (n = 31) | | | | | | |
| Non- | Sensitive | 31% | (5) | 6% | (1) | 12% | (2) |
| chromothripsis | (n = 16) | | | | | | |
| | Resistance | 71% | (22) | 29% | (9) | 42% | (13) |
| | (n = 31) | | | | | | |

¹Defective MMR: SBS6, SBS20, SBS26, SBS44
²Defective BER: SBS18, SBS30, SBS36
³Defective HRR: SBS3

### Discussion

Preexisting tumor heterogeneity and accelerated diversification in response to targeted therapy are thought to fuel the evolution of acquired resistance. Here, we provide insights into the underlying genomic instability processes that generate preexisting and therapy-elicited, de novo clonal diversification in advanced cutaneous melanoma. We identified chromothripsis as well as ecDNAs and CGRs as highly recurrent genomic SVs in MAPKi-naive/sensitive and acquired MAPKi-resistant melanoma, both in the clinical setting (BRAF^{V600MUT} melanoma, where MAPKi therapy is a standard-of-care therapy) and the experimental setting (NRAS^{MUT} melanoma, where there is a lack of targeted therapy option). CGRs can derive from re-integration of ecDNAs or through breakage-fusion-bridge (BFB) cycles (20,43). We did not detect any BFB event in any of our tumors, MAPKi-sensitive/naive or acquired-resistant, favoring ecDNA integration as the main route of CGR generation in the setting of metastatic cutaneous melanoma. Consistently, the acute stress of MAPKI therapy favors ecDNAs, while stable or chronic stress favors re-integration of ecDNAs into chromosomes as HSRs (13). Moreover, following chromothripsis, chimeric circularization of DNA and re-integration of DNA circles into chromosomes constitute a major source of SVs and linear genome mutagenesis (44,45). Importantly, the selective pressure of MAPKi therapy is evidenced by the amplification of bona fide resistance-driver genes via ecDNAs and CGRs. The report here of resistance-specific ecDNAs and CGRs amplifying a wide array of coding and non-coding sequences warrants future investigations into their resistance-causative mechanisms.

Even though chromothripsis is regarded as a potential precursor of ecDNAs, it creates oscillating copy numbers of genomic segments but does not cause high-level amplification. We found that, within each tumor, ecDNAs (mean total size per genome, 7 MB; mean size per ecDNA, 343 kb) and their re-integrated counterparts, CGRs (mean total size per genome, 6 Mb; mean size per CGR, 598 kb), almost always span genomic regions bounded by larger chromothriptic regions (mean total size per genome, 474 Mb; mean size per chromothriptic region, 120 Mb), which is consistent with a chromothriptic origin of ecDNA- and CGR-amplicons. Chromothripsis can occur as a result of micronuclei formation around lagging chromosomes or chromosome bridge formation due to telomere crisis (46-48). Both aberrant processes are associated with a loss of primary or micronuclear membrane integrity and subsequent mutagenesis. This is consistent with our finding of enhanced mutational density within chromothriptic genomic regions, especially within acquired-resistant genomes, as well as a resistance-specific mutator phenotype enriched for signatures of excessive single-stranded DNA damage and/or deficient repair (BER and MMR). Intriguingly, a recent study proposed that a specific BER defect may predispose micronuclei-associated or cytoplasmic chromosomes to breakage, a key step toward chromothripsis (49).

Translationally, we produced in vivo evidence supportive of DNA-PK_{CS}′ well-studied role in NHEJ as critical in promoting the total segment sizes of ecDNAs and CGRs generated early on MAPKi therapy. This finding is consistent with prior literature supporting NHEJ as key to ecDNA formation (28,50). Selection for ecDNAs and CGRs is considered a direct mechanism promoting tumor fitness in response to a given stressor. Thus, therapy- or stressor-specific ecDNAs and CGRs can be considered mechanisms promoting tumor fitness, agnostic of the specific molecular resistance pathway. Therefore, reduction of the total ecDNA and CGR genomic spans by DNA-PKi co-treatment may serve as a mechanistic or pharmacodynamic marker for its combinatorial efficacy in preventing or delaying acquired MAPKi-resistance.

DNA-PK_{CS}, the target of DNA-PKi, subserves other less characterized cancer survival pathways (37). It is possible that another beneficial mechanism of action of DNA-PKi co-exists. DNA-PKi has been proposed in combination with agents that directly induce DNA damage, such as radiotherapy or chemotherapy, with the intent of radio- or chemo-sensitization (37). The rationale is based on catastrophic DSBs that would result in excessive DNA damage repair stress and hence the synergistic induction of death in cancer cells, especially those over-expressing DNA-PK_{CS}. Here, we rationalize the combination of DNA-PKi and MAPKi based on dual concepts. First, the rapid induction of genomic instability mechanisms, in particular the generation of ecDNA- and CGR-amplicons, is critical for genomic diversification and perhaps epigenomic re-programming necessary for melanoma to adapt quickly to MAPKi therapy. In this rationale, DNA-PKi suppresses NHEJ, which is necessary for the efficient formation of ecDNAs and CGRs. Second, MAPKi is potentially an inducer of DNA damage and/or DNA damage repair deficiency. In this context, MAPKi has been shown to induce DNA damage in early drug-tolerant persister subpopulations (42). A therapy-induced oxidative metabolic adaptation (51) has been proposed to cause ROS-induced mutagenesis, which can be repaired by DNA single-stranded break (SSB) repair processes such as BER and MMR. DNA-PK_{CS}, in addition to DSB repair, can also bind to and is activated by DNA SSBs (52). Excessive DNA SSBs can be converted into DNA DSBs, engendering DNA damage repair stress and/or chromosome breakage. The latter may be a pathway to chromothripsis and ecDNA/CGR generation (49).

Our demonstration that ecDNAs and CGRs drive acquired MAPKi-resistance advances the concept that multiple resistance mechanisms, genetic and epigenetic, as well as direct (drug-target or MAPK pathway reactivation) and indirect (non-drug-target pathway activation), are simultaneously causal of clinically acquired resistance. Future work needs to dissect this hybrid genomic-epigenomic model with ecDNAs and CGRs at the center of therapeutic targeting efforts. Findings here also advance the concepts that preventing, instead of reversing, acquired resistant phenotypes may be more impactful clinically and that targeting DNA-PK_{CS} and NHEJ, and potentially MMEJ and HRR, lies at the center of this approach in stabilizing cancer genomes during oncogene-targeted therapies.

### REFERENCES

1. Flaherty K T, et al. Inhibition of mutated, activated BRAF in metastatic melanoma. N Engl J Med 2010:363:809-19
2. Flaherty K T, et al. N Engl J Med 2012
3. Nazarian R, et al. Nature 2010; 468:973-7
4. Shi H, et al. Cancer Discov 2012; 2:414-24
5. Shi H, et al. Nat Commun 2012; 3:724
6. Long G V, et al. J Clin Oncol 2018; 36:667-73
7. Hugo W, S et al. Cell 2015; 162:1271-85
8. Moriceau G, et al. Cancer Cell 2015; 27:240-56
9. Shi H, et al. Cancer Discov 2014; 4:80-93
10. Dummer R, et al. Lancet Oncol 2017; 18:435-45
11. Hong A, et al. Cancer Discov 2021; 11:714-35
12. Cortes-Ciriano I, et al. Nat Genet 2020; 52:331-41
13. Song K, et al. Cancer Discov 2022:12:1046-69
14. Nathanson D A, et al. Science 2014; 343:72-6
15. Tumer K M, et al. Nature 2017; 543:122-5
16. Helmsauer K, et al. Nat Commun 2020; 11:5823
17. Hung K L, et al. Nature 2021; 600:731-6
18. Wu S, Bafna V, Mischel P S. Curr Opin Genet Dev 2021:66:78-82
19. Wu S, et al. Nature 2019; 575:699-703
20. Shoshani O, et al. Nature 2021; 591:137-41
21. Van Allen E M, et al. Cancer Discov 2014:4:94-109
22. Hong A, et al. Cancer Discov 2018; 8:74-93
23. Chadee D N, et al. Proc Natl Acad Sci USA 2006; 103:4463-8
24. Hung K L, et al. Nat Genet 2022
25. Morton A R, et al. Cell 2019; 179:1330-41 e13
26. Zhu Y, et al. Cancer Cell 2021; 39:694-707 e7
27. Fishilevich S, et al. Database (Oxford) 2017; 2017
28. Rausch T, et al. Cell 2012; 148:59-71
29. Stephens P J, et al. Cell 2011; 144:27-40
30. Zhang C Z, et al. Nature 2015; 522:179-84
31. Krupina K, Goginashvili A, Cleveland D W. Curr Opin Cell Biol 2021; 70:91-9
32. Alexandrov L B, et al. Nature 2020; 578:94-101
33. Bergstrom E N, et al. Nature 2022; 602:510-7
34. Chang H H Y, et al. Nat Rev Mol Cell Biol 2017; 18:495-506
35. Korbel J O, Campbell P J. Cell 2013; 152:1226-36
36. Maher C A, Wilson R K. Cell 2012; 148:29-32
37. Dylgjeri E, Knudsen K E. Cancer Res 2022; 82:523-33
38. Patterson-Fortin J, D'Andrea A D. Cancer Res 2020; 80:4593-600
39. Sharma S V, et al. Cell 2010; 141:69-80
40. Song C, et al. Cancer Discov 2017; 7:1248-65
41. Hamor S J, Brennan A, Cano C. ChemMedChem 2017; 12:895-900
42. Yang C, et al. Nat Commun 2021; 12:1747
43. Cowell J K, Miller O J. Chromosoma 1983; 88:216-21
44. Koche R P, et al. Nat Genet 2020; 52:29-34
45. Rosswog C, et al. Nat Genet 2021; 53:1673-85
46. Ly P, Cleveland D W. Trends Cell Biol 2017; 27:917-30
47. Ly P, et al. Nat Cell Biol 2017; 19:68-75
48. Maciejowski J, Li Y, Bosco N, Campbell P J, de Lange T. Cell 2015; 163:1641-54
49. Tang S, Stokasimov E, Cui Y, Pellman D. Nature 2022; 606:930-6
50. Kim H, et al. Nat Genet 2020; 52:891-7
51. Labrie M, et al. Nat Rev Cancer 2022; 22:323-39
52. Hammarsten O, DeFazio L G, Chu G. J Biol Chem 2000; 275:1541-50
53. Li H, Durbin R. Bioinformatics 2009; 25:1754-60
54. Li H, et al. Bioinformatics 2009; 25:2078-9
55. Talevich E, et al. PLoS Comput Biol 2016; 12:e1004873
56. Miller C A, et al. PLoS One 2011; 6:e16327
57. Wala J A, et al. Genome Res 2018; 28:581-91
58. Eisfeldt J, et al. F1000Res 2017; 6:664
59. Rausch T, et al. Bioinformatics 2012; 28:i333-i9
60. Deshpande V, et al. Nat Commun 2019; 10:392
61. Kim S, et al. Nat Methods 2018; 15:591-4
62. Blokzijl F, et al. Genome Med 2018; 10:33
63. Khan A, Zhang X. Nucleic Acids Res 2016; 44:D164-71
64. Dobin A, et al. Bioinformatics 2013; 29:15-21
65. Liao Y, Smyth G K, Shi W. Bioinformatics 2014; 30:923-30
66. Robinson M D, Oshlack A. Genome Biol 2010:11:R25
67. Shi H, et al. Cancer Discov 2014; 4:69-79

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of enhancing anti-melanoma therapy in a subject in need thereof, the method comprising administering to the subject an effective amount of a DNA-PK_{CS} inhibitor (DNA-PKi) and/or a PARP1/2 inhibitor (PARPi).

2. The method of claim 1, wherein the DNA-PKi is NU7026 and/or AZD7648.

3. The method of claim 1, wherein the PARPi is ABT888 (veliparib), olaparib, iniparib, niraparib, talazoparib, AZD2461, and/or rucaparib.

4. The method of claim 1, wherein the administering comprises administering both a DNA-PKi and a PARPi.

5. The method of claim 1, wherein the subject is treated with one or more mitogen-activated protein kinase (MAPK) inhibitors, and optionally, one or more anti-PD-1/L1 antibodies as anti-melanoma therapy.

6. The method of claim 5, wherein the MAPK inhibitor(s) or MAPK inhibitor(s) plus anti-PD-1/L1 antibodies is administered concomitantly with, prior to, and/or subsequent to the administering of the DNA-PKi and/or PARPi.

7. The method of claim 5, wherein the MAPK inhibitor is selected from: a type I RAF inhibitor; a MEK inhibitor; a type II RAF inhibitor, a pan-RAF inhibitor, a KRAS-G12C inhibitor; and a combination of the above.

8. The method of claim 7, wherein the type I RAF inhibitor is vemurafenib, dabrafenib, and/or encorafenib.

9. The method of claim 7, wherein the MEK inhibitor is trametinib, bbinimetinib, and/or cobimetinib.

10. The method of claim 7, wherein the pan-RAF inhibitor is BGB-283, BGB-3245, DAY101TAK-580, KIN-2787, and/or LXH254.

11. The method of claim 7, wherein the KRAS-G12C inhibitor is sotorasib or adagrasib.

12. The method of claim 7, wherein the combination is a type I RAF inhibitor and a MEK inhibitor, or a type II RAF inhibitor, and a MEK inhibitor.

13. A method of preventing or inhibiting acquired resistance to MAPK inhibitor (MAPKi) therapy in a subject in need thereof, the method comprising administering to the subject an effective amount of a DNA-PKi and/or a PARPi.

14. A method of inhibiting chromothripsis in a subject in need of MAPK inhibitor (MAPKi) therapy, the method comprising administering to the subject an effective amount of a DNA-PKi and/or a PARPi.

15. The method of claim 13, wherein the DNA-PKi and/or PARPi is administered concomitantly with a MAPKi.

16. The method of claim 14, wherein the DNA-PKi and/or PARPi is administered non-continuously.

17. The method of claim 13, wherein the DNA-PKi is NU7026 and/or AZD7648.

18. The method of claim 13, wherein the PARPi is ABT888 (veliparib), olaparib, iniparib, niraparib, talazoparib, AZD2461, and/or rucaparib.

19. The method of claim 13, wherein the administering comprises administering both a DNA-PKi and a PARPi.

20. The method of claim 13, wherein the MAPK inhibitor is selected from: a type I RAF inhibitor; a MEK inhibitor; a type II RAF inhibitor, a pan-RAF inhibitor, a KRAS-G12C inhibitor; and a combination of the above.


SEQUENCE LISTING (12 sequences)

SEQ ID NO: 1 - DNA, 19 bp, synthetic construct
  source 1..19: mol_type = other DNA
cagtgaaagtctgaatcat

SEQ ID NO: 2 - DNA, 19 bp, synthetic construct
  source 1..19: mol_type = other DNA
gtcatggattcaagaaata

SEQ ID NO: 3 - DNA, 19 bp, synthetic construct
  source 1..19: mol_type = other DNA
gagcttacatgctaatgta

SEQ ID NO: 4 - DNA, 19 bp, synthetic construct
  source 1..19: mol_type = other DNA
ggtcatggattcaagaaat

SEQ ID NO: 5 - DNA, 21 bp, synthetic construct
  source 1..21: mol_type = other DNA
ttcgacgccacaccgtttatt

SEQ ID NO: 6 - DNA, 21 bp, synthetic construct
  source 1..21: mol_type = other DNA
tatgtcagtggactaatggat

SEQ ID NO: 7 - DNA, 59 bp, synthetic construct
  source 1..59: mol_type = other DNA
ctactcggaaggctgagacaggcgatcgcttgaacccaggaggtggaggttgcagcaag

SEQ ID NO: 8 - DNA, 60 bp, synthetic construct
  source 1..60: mol_type = other DNA
tgtgaaagtacaaaatgagtgtagagcaggggttccaacctccaggccacagactggtac

SEQ ID NO: 9 - DNA, 63 bp, synthetic construct
  source 1..63: mol_type = other DNA
cttctattgaaataaatggctttactaaatcaagtttctcagaagcttccagagtagcttttg

SEQ ID NO: 10 - DNA, 63 bp, synthetic construct
  source 1..63: mol_type = other DNA
aatcaaagaagtaaactaaaaatgcaaatttaatttcaggttcattacaaatggtctgtattt

SEQ ID NO: 11 - DNA, 62 bp, synthetic construct
  source 1..62: mol_type = other DNA
tcccagaatttcctcttgccttcccctatggaaccttcacctatggcacattcagaactttc

SEQ ID NO: 12 - DNA, 62 bp, synthetic construct
  source 1..62: mol_type = other DNA
tcaattaccagttacctcccagaatttcctcttgccttcccctatggaaccttcacctatgg